(19) United States
(12) Patent Application Publication
(10) Pub. No.: US 2026/0347122 A1
(43) Pub. Date: Nov. 5, 2026

(54) SYSTEMS, METHODS, AND COMPONENTS FOR PACKAGE HANDLING AND SORTATION

(71) Applicant: Aegis Sortation, LLC, Louisville, KY (US)

(72) Inventors: Dean TERRELL, Louisville, KY (US); Jeffrey Paul HENLEY, Louisville, KY (US); Lance GATEWOOD, Louisville, KY (US); Anthony J. YOUNG, Louisville, KY (US); Kevin RANEY, Louisville, KY (US); Robert BROWDER, Louisville, KY (US); Marvin Gregory WHITLOCK, Louisville, KY (US); Scott CRANCE, Louisville, KY (US); Ken ICE, Louisville, KY (US); Michael KARAGLANIS, Louisville, KY (US); Brian YOUNT, Louisville, KY (US); Barry SWEATT, Louisville, KY (US); Thomas M. PHILLIPS, Louisville, KY (US); Logan YOUNG, Louisville, KY (US); Derek Robert STRUM, Louisville, KY (US); David Wesley CALDWELL, Louisville, KY (US); James Thomas HIND, Louisville, KY (US)

(73) Assignee: Aegis Sortation, LLC, Louisville, KY (US)

(21) Appl. No.: 19/218,256

(22) Filed: May 24, 2025

Related U.S. Application Data
(63) Continuation-in-part of application No. 18/419,410, filed on Jan. 22, 2024, now Pat. No. 12,690,841.
Application No. 18/419,410 is a continuation-in-part of application No. 18/399,000, filed on Dec. 28, 2023, now Pat. No. 12,312,180.
Application No. 18/399,000 is a continuation of application No. 18/092,226, filed on Dec. 31, 2022, now Pat. No. 12,012,290.
Application No. 18/092,226 is a continuation-in-part of application No. 17/843,313, filed on Jun. 17, 2022, now Pat. No. 11,969,759.
Application No. 17/843,313 is a continuation of application No. 17/566,527, filed on Dec. 30, 2021, now Pat. No. 11,731,169.
(60) Provisional application No. 63/216,340, filed on Jun. 29, 2021.
(60) Provisional application No. 63/440,439, filed on Jan. 22, 2023.
(60) Provisional application No. 63/456,088, filed on Mar. 31, 2023.
(60) Provisional application No. 63/539,884, filed on Sep. 22, 2023.
(60) Provisional application No. 63/543,450, filed on Oct. 10, 2023.
(60) Provisional application No. 63/651,989, filed on May 25, 2024.

(51) Int. Cl. B65G 47/19; B65G 47/26; B65G 47/48

(52) U.S. Cl. CPC B65G 47/19; B65G 47/268; B65G 47/48; B65G 2201/0285; B65G 2203/0241; B65G 2203/044

(57) ABSTRACT

A chute tendering system is provided including a chute and a pair of bidirectional conveyors each positioned along a facet of the chute between an upper opening of the chute and a lower opening of the chute. A sensor may sense, for example, a velocity, direction, or frequency of parcels within the chute and may be operatively connected to a controller which may control operation of the bidirectional conveyors based on information output from the sensor. The system may additionally include one or more further mechanical means for directing parcels within the chute or exiting from the chute.

### CROSS-REFERENCE TO RELATED APPLICATIONS

This present Application claims the benefit of U.S. Provisional Application 63/651,989 filed May 25, 2024 and is a Continuation-in-part of U.S. application Ser. No. 18/419,140 filed Jan. 22, 2024.

U.S. application Ser. No. 18/419,140 claims the benefit of U.S. Provisional Applications 63/440,439, filed Jan. 22, 2023; 63/539,884; 63/456,088, filed Sep. 22, 2023; and 63/543,450, filed Oct. 10, 2023.

U.S. application Ser. No. 18/419,140 is also a continuation in part of U.S. application Ser. No. 18/399,000 filed Dec. 28, 2023, which is a continuation of U.S. application Ser. No. 18/092,226 filed Dec. 31, 2022, which is a continuation in part of U.S. application Ser. No. 17/843,313 filed Jun. 17, 2022, which is a continuation of U.S. application Ser. No. 17/566,527 filed Dec. 30, 2021, which claims the benefit of U.S. Provisional Application 63/216,340, filed Jun. 29, 2021 in the United States Patent Office.

The disclosures of all of the above-identified Applications, including all attachments thereto, are incorporated herein by reference in their entireties.

### BACKGROUND

### 1. Field

Apparatuses and methods consistent with example embodiments relate to sortation conveyor systems where smaller packages are accumulated into groups of packages in an automated consolidated bagging system; to sortation systems and methodologies that divert loose small packages into bags, which can then be closed; to systems and methods that provide automation for transporting of container to/from designated areas; and to a system, components and methodology for chute tendering and container fill applications.

Apparatuses and methods consistent with example embodiments may be applicable to any and all systems and methodologies disclosed in the above-referenced related applications.

### 2. Description of Related Art

Related art automated sorters of smaller packages (hereinafter “smalls sorters”) used in conventional sortation systems divert loose small packages into bags or other containers (hereinafter simply referred to as “bags”), to be accumulated. Once a specified number of packages accumulate in a bag, the bag is then logically and physically closed, and all of the identifications (IDs) and information associated with the packages are associated with the bag and are logically stored. Then, a label is printed and applied to the outside of the bag so the packages in the bag can be tracked as a group within the bag, where all such packages are associated with the bag ID. However, such smalls sorters may be undesirably less effective and less efficient because packages often miss the bag they are intended for, resulting in a miss-sorted package. This may require the use of additional material, such as an additional bag, and may require manual intervention, for example, to monitor and close the bag and then to move the bag for further processing on a conveyor system.

### SUMMARY OF EXAMPLE EMBODIMENTS

Example embodiments may address at least the above problems and/or disadvantages and other disadvantages not described above. Also, example embodiments are not required to overcome the disadvantages described above, and may not overcome any of the problems described above.

According to an aspect of an example embodiment, a chute tendering system may comprise: chute comprising a passage extending from an upper opening to a lower opening, wherein a cross-sectional area of the lower opening is smaller than a cross-sectional area of the upper opening; a pair of bidirectional conveyors, each disposed along a facet of the chute between the upper opening and the lower opening.

According to an example implementation, the chute tendering system may further comprise: at least one rod movably connected to the chute such that the at least one rod is movable between a first position in which it is not positioned within a path of parcels within the chute and a second position in which the rod extends into the path to thereby contact one or more parcels within the chute.

According to an example implementation, the chute tendering system may further comprise: a pair of flaps movably connected to the chute, wherein each of the pair of flaps is moveable between a retracted position outside of a path of parcels exiting the lower opening of the chute and an extended position intersecting the path to thereby guide one or more parcels exiting the lower opening of the chute.

According to an example implementation, the chute tendering system may further comprise: a parcel jam detection system comprising: at least one sensor configured to detect at least one of a velocity, a frequency, and a direction of movement of packages within the chute; a controller comprising a non-volatile memory storing instructions thereon, and a processor configured to execute the instructions, wherein the controller is operatively connected to the pair of bidirectional conveyors and to the at least one sensor and is configured to execute the instructions to thereby: receive information output from the at least one sensor, and control operation of the pair of bidirectional conveyors based on the information output from the at least one sensor.

According to an example implementation, the controller may be further configured to: determine when a parcel jam has been cleared based on the information output from the at least one sensor, and output a notification upon a determine that a parcel jam has been cleared.

According to an aspect of an example embodiment, an automated consolidated bagging system may comprise: a plurality of collector conveyors; a bagging conveyor; a plurality of feed conveyors, each positioned to receive parcels from a corresponding one of the plurality of collector conveyors and output the parcels onto the bagging conveyor; at least one sensor configured to sense one of a volume and a number of parcels disposed on each of the plurality of feed conveyors; a controller comprising a non-volatile memory storing instructions thereon, and a processor configured to execute the instructions, wherein the controller is operatively connected to each of the plurality of feed conveyors and to the at least one sensor and is configured to execute the instructions to thereby: receive information output from the at least one sensor, and control operation of each of the plurality of feed conveyors based on the information output from the at least one sensor.

According to an aspect of an example embodiment, a bag exception system may comprise: a take away conveyor configured to convey bags thereon; a diverter configured to divert one or more bags off the take away conveyor; a reader configured to read data from the bags on the take away conveyor a controller comprising a non-volatile memory storing instructions thereon, and a processor configured to execute the instructions, wherein the controller is operatively connected to the reader and to the diverter and is configured to execute the instructions to thereby: receive information output from the reader, and control operation of the diverter based on the information output from the reader.

According to an example implementation, the reader may comprise one of a camera configured to obtain an image of information on the bags and a radio frequency reader configured to read radio frequency identification (RFID) tags connected to the bags.

### BRIEF DESCRIPTION OF THE DRAWINGS

The above and/or other implementations will become apparent and more readily appreciated from the following description of example embodiments, taken in conjunction with the accompanying drawings, in which:

FIGS. 1A and 1B, combined, are a flow chart of an automated sortation method according to one or more example embodiments;

FIGS. 2A and 2B illustrate a top view and a side view, respectively, of an automated sortation system according to one or more example embodiments;

FIG. 3A is a perspective view of an automated sortation system according to one or more example embodiments;

FIG. 3B is a schematic illustration of an automated sortation system according to an example embodiment, FIG. 3C shows an enlarged view of a section of FIG. 3B, according to an example embodiment, and FIG. 3D shows an enlarged view of another section of FIG. 3B, according to one or more example embodiments;

FIG. 4 is an enlarged perspective view of a section of FIG. 3A and FIG. 3B, according to one or more example embodiments;

FIGS. 5A, 5B, and 5C are an enlarged schematic view, a side view, and a back view, respectively, of an end of a cleated conveyor, a hopper, and chutes of a system according to one or more example embodiments;

FIGS. 5D, 5E, 5F, and 5G are a perspective view, a top view, an end view, and a side view, respectively, of a cleated conveyor, a hopper, chutes, and carousels of a system according to one or more example embodiments;

FIGS. 5H, 5I, and 5J are a side view, a perspective view, and a bottom view of an example carousel according to one or more example embodiments;

FIGS. 6A, 6B, and 6C are different perspective views of a smart bin according to one or more example embodiments;

FIG. 6D is a plan view of the front of a smart bin according to one or more example embodiments;

FIG. 6E is a plan view of a side of the smart bin according to one or more example embodiments;

FIGS. 7A, 7B, 7C, 7D, 7E, and 7F show different views of an example smart bin according to one or more example embodiments;

FIGS. 8A, 8B, 8C, 8D, and 8E are a perspective view, a side view, a front view, a detail of back view, and a back view, respectively, of an example hopper according to one or more example embodiments;

FIGS. 9A and 9B are an exploded view, an exploded view with the guard hidden of an example hopper according to one or more example embodiments;

FIG. 9C shows a detail of a portion of FIG. 9B;

FIG. 10 is a schematic illustration of a main control panel according to one or more example embodiments;

FIGS. 11A, 11B, 11C, and 11D are, respectively, perspective, top, side and front views of a two-way linear rail assembly according to one or more example embodiments;

FIGS. 12A, 12B, 12C, and 12D are, respectively, perspective, top, side and front views of a three-way linear rail assembly according to one or more example embodiments;

FIGS. 13A, 13B, 13C, and 13D are, respectively, perspective, exploded perspective, side, and cross sectional (along cross section line of FIG. 13C) views of a hopper assembly according to one or more example embodiments;

FIGS. 14A, 14B, 14C, 14D, and 14E are, respectively, a plan view and four different cross section views (along labeled cross section lines of FIG. 14A) of a system according to one or more example embodiments;

FIGS. 15A, 15B, 15C, 15D, 15E, 15F, 15G, and 15H are, respectively, an isometric view, a plan view and six different cross section views (along labeled cross section lines of FIG. 15B) of a system according to one or more example embodiments;

FIGS. 16A, 16B, 16C, 16D, and 16E are, respectively, an isometric view, enlarged schematic plan view and various cross section and enlarged views, of a system according to one or more example embodiments;

FIGS. 17A, 17B, and 17C are, respectively, an isometric view, a top view and a side view of a smart bin according to one or more example embodiments;

FIG. 18A illustrates components of a zipper structure according to one or more example embodiments;

FIG. 18B is a block diagram of a system implementing an auto bagging configuration according to one or more example embodiments;

FIGS. 19A, 19B, 19C, 19D, and 19E, illustrate a device for containers requiring a zipping process according to one or more example embodiments;

FIGS. 20A and 20B, diagrammatically illustrate containers requiring manipulation according to one or more example embodiments;

FIGS. 21A, 21B, 21C, 21D, 21E, 21F, 21G, and 21H, illustrate devices and processes for containers requiring manipulation and/or closure according to one or more example embodiments;

FIGS. 22A and 22B illustrate devices for handling of containers requiring a zipping process according an example embodiment;

FIGS. 22C and 22D illustrate alternative devices for handling of containers requiring a zipping process according an example embodiment;

FIGS. 23A, 23B, 23C, 23D, and 23E, illustrate devices and methodologies for handling and manipulating containers according to one or more example embodiments;

FIGS. 24A, 24B, 24C, and 24D, illustrate devices and methodologies for handling and manipulating containers according to one or more example embodiments;

FIG. 25A illustrates a partial view of a system implementing the same, according to one or more example embodiments;

FIGS. 25B, 25C, and 25D, illustrate a side view, a top view, and an isometric view, respectively, of the system of FIG. 25A, according to one or more example embodiments;

FIGS. 26A, 26B, 26C, and 26D, illustrate devices, methodologies, and systems according to one or more example embodiments;

FIGS. 27A, 27B, and 27C, illustrate devices, methodologies, and systems according to one or more example embodiments;

FIG. 28 illustrates configuration of devices for handling of containers according to one or more example embodiments;

FIG. 29 illustrates devices and methodologies, and a partial view of a system according to one or more example embodiments;

FIGS. 30A and 30B are isometric detailed views of devices and methodologies according to one or more example embodiments;

FIGS. 31A, 31B, 31C, and 31D, diagrammatically illustrate examples of devices and methodology, according to one or more example embodiments;

FIG. 32 diagrammatically illustrates in an enlarged view an example of some of the features of devices illustrated, for example, in FIGS. 30A and 30B, and FIGS. 31A, 31B, 31C, and 31D;

FIGS. 33A, 33B, 33C, and 33D diagrammatically illustrate examples of devices and methodology, and partial views of systems implementing the same, according to one or more example embodiments;

FIGS. 34A and 34B diagrammatically illustrate examples of devices and methodology according to one or more example embodiments;

FIG. 35 diagrammatically illustrates a side view of a device according to one or more example embodiments;

FIG. 36 diagrammatically illustrates in an isometric view an example of a configuration of multiple devices, according to one or more example embodiments;

FIG. 37 diagrammatically illustrates examples of devices and methodology, according to one or more example embodiments;

FIG. 38 diagrammatically illustrates examples of devices and methodology, and partial views of systems implementing the same, according to one or more example embodiments;

FIG. 39 diagrammatically illustrates examples of devices and methodology according to one or more example embodiments;

FIG. 40 diagrammatically illustrates examples of devices and methodology according to one or more example embodiments;

FIG. 41 diagrammatically illustrates in an isometric view examples of devices and methodology according to one or more example embodiments;

FIG. 42 diagrammatically illustrates an example of a system configuration including containers;

FIG. 43 diagrammatically illustrates examples of devices and methodology, and partial views of systems implementing the same, according to one or more example embodiments;

FIG. 44 diagrammatically illustrates examples of devices and methodology according to one or more example embodiments;

FIG. 45 diagrammatically illustrates in an isometric view examples of devices and methodology according to one or more example embodiments;

FIG. 46 schematically illustrates in an isometric view an example of a device according to one or more example embodiments;

FIGS. 47A and 47B diagrammatically illustrate partial views of examples of devices and methodology, according to one or more example embodiments;

FIGS. 48A and 48B diagrammatically illustrate top and isometric views, respectively, of examples of devices and methodology, according to one or more example embodiments;

FIGS. 49A, 49B, 49C, 49D, and 49E diagrammatically illustrate details of examples of devices, as well as applicable methodology, according to one or more example embodiments;

FIG. 50 is a block diagram illustrating example of certain features of a system and devices according to one or more example embodiments;

FIG. 51A diagrammatically illustrates in an isometric view and example of a device and partial view of a system according to one or more example embodiments;

FIGS. 51B, 51C, and 51D illustrate examples of top view, side view, and end view, respectively, of a device of FIG. 51A;

FIGS. 52A and 52B diagrammatically illustrate examples of devices having a certain alternative configuration, according to one or more example embodiments;

FIGS. 53A, 53B, 53C, and 53D diagrammatically illustrate examples of a system and methodology, according to one or more example embodiments;

FIGS. 54A, 54B, and 54C diagrammatically illustrate an example of features and

operation of devices, and associated methodology, according to one or more example embodiments;

FIGS. 55A and 55B diagrammatically illustrate an example of features and operation of devices, and associated methodology, according to one or more example embodiments;

FIGS. 56A, 56B, and 56C diagrammatically illustrate in perspective views examples of features and operation of devices, and associated methodology, according to one or more example embodiments;

FIG. 57 diagrammatically illustrate an example of features of a device, and a partial view of certain system components, according to one or more example embodiments;

FIGS. 58A and 58B diagrammatically illustrate an example of features and operation of a system, and associated methodology, according to one or more example embodiments;

FIGS. 59A, 59B, and 59C diagrammatically illustrate in partial enlarged views examples of features and operation of a system, and associated methodology, according to one or more example embodiments;

FIGS. 59D, 59E, 59F and 59G diagrammatically illustrate in partial enlarged views examples of features and operation of a system, and associated methodology, according to one or more example embodiments;

FIG. 60 diagrammatically illustrates an example of features and operation of system and devices, and associated process flow, according to one or more example embodiments;

FIGS. 61A, 61B, and 61C together schematically illustrate an example of features of a device, according to one or more example embodiments;

FIGS. 62A, 62B, 62C, and 62D diagrammatically illustrate an example of features and operation of a device, and associated methodology, according to one or more example embodiments;

FIGS. 63A, 63B, 63C, and 63D diagrammatically illustrate an example of features and operation of another device, and associated methodology, according to one or more example embodiments;

FIGS. 64A and 64B are a perspective view and a side view, respectively, of a chute tendering system according to an example embodiment;

FIGS. 65A-65E illustrate a system for an ACB/SmartBagger bilateral AB splitter according to one or more example embodiments;

FIG. 66 is a perspective view of system of conveyors, according to one or more example embodiments; and

FIGS. 67A, 67B, 68A, 68B, and 69 diagrammatically illustrate an example of features and operation of another device, and associated methodology, that can be, but is not required to be implements in, or with, systems and methodologies according to one or more example embodiments of the present disclosure.

### DETAILED DESCRIPTION

Reference will now be made in detail to example embodiments which are illustrated in the accompanying drawings, wherein like reference numerals refer to like elements throughout. In this regard, the example embodiments may have different forms and may not be construed as being limited to the descriptions set forth herein.

It will be understood that the terms “include,” “including,” “comprise,” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list.

Expressions of relational orientation, such as “upper,” “lower,” “inside,” “outside,” etc. which are used for explaining the structural positions of various components as described herein, are not absolute but relative. The orientation expressions are appropriate when the various components are arranged as shown in the figures, but should change accordingly when the positions of the various components in the figures change.

Expressions of relational orientation, such as “upper,” “lower,” “inside,” “outside,” etc. which are used for explaining the structural positions of various components as described herein, are not absolute but relative. The orientation expressions are appropriate when the various components are arranged as shown in the figures, but should change accordingly when the positions of the various components in the figures change.

Various terms are used to refer to particular system components. Different companies may refer to a component by different names—this document does not intend to distinguish between components that differ in name but not function.

Matters of these example embodiments that are obvious to those of ordinary skill in the technical field to which these example embodiments pertain may not be described herein in detail.

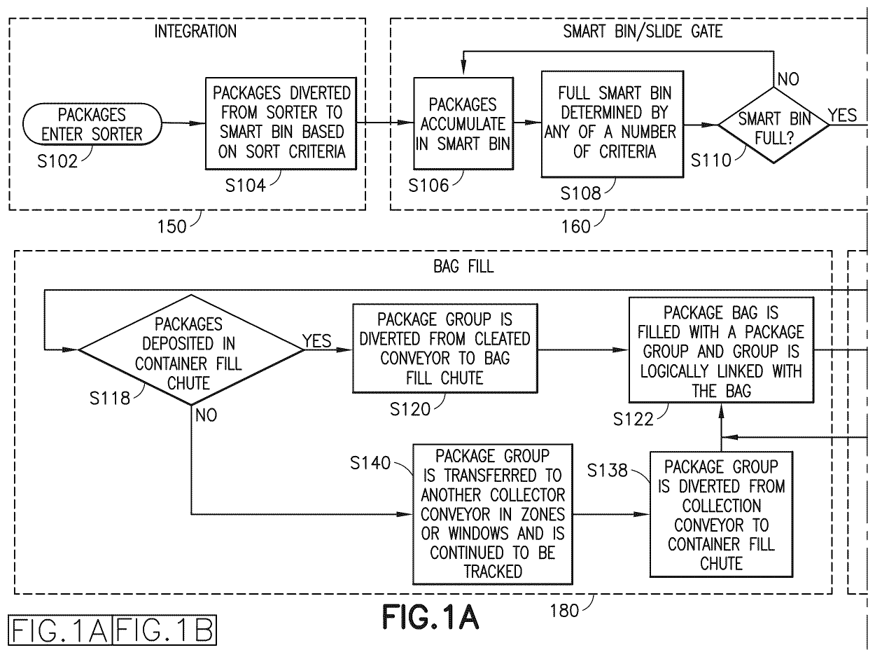

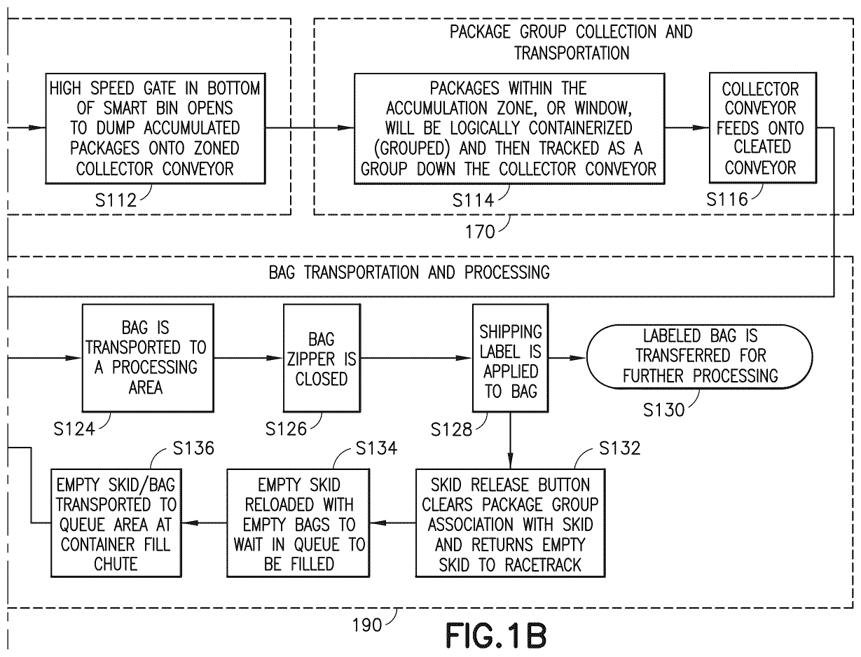

FIG. 1 is a flow chart of an automated sortation method according to an example embodiment. As shown in FIG. 1, various operations of an automated sortation method occur in an integration section 150, at which packages from another sorter are received and diverted to a smart bin/slide gate section 160. From the smart bin/slide gate section 160, packages are transferred to a package group collection and transportation section 170, then to a bag fill section 180, and then to a bag transportation and processing section 190. It should be noted that while the term “bag” is used herein, this term is not limiting, and any example embodiment described herein may be used in conjunction with any suitable container, including, but not limited to a bag.

According to the example embodiment shown in FIG. 1, packages enter a sorter in the integration section (S102), and are diverted from the sorter into a smart bin (S104), based on, for example an established sort criteria. In the smart bin/slide gate section 160, packages may continue to accumulate in the smart bin (S106). A determination of whether the smart bin is full (S108, S110) may be made based on a number of packages that have accumulated therein; based on a signal from an optical device, such as a photocell or other optical sensor, which detects a full condition; and/or based on one or more volumetric calculations based on known or sensed volumes of the packages. For example, there may be a smart-bin controller, and data on dimensions of each received package may be transmitted to the smart-bin controller, such that a total volume of packages can be calculated and tracked, and when a threshold volume is reached, the smart bin may be considered full and ready for release. Alternately, a photocell may be disposed at a fixed location on the smart bin or with respect to the smart bin, and when a signal from the photocell indicates that the photocell has been blocked for a predetermined period of time, it may be determined that the smart bin is full and ready to release.

According to another example implementation, when a smart bin is determined to be full, the system can be configured to release the packages onto the collector conveyor according to one or more of various criteria including, but not limited to: a leading edge of a tracking window being a certain distance (for example, 18 inches) past a configured offset of chute of a smart bin (this distance being configured to prevent packages from overflowing into a next zone when they are released); and a tracking window being is available and not assigned to another chute of another smart bin.

If it is determined that a smart bin is full (S110—YES), packages in the smart bin are transferred to a zoned collector conveyor (S112).

In an example implementation, the smart bin can include a gate, for example a high speed gate, at a bottom thereof, such that when the smart bin is full (S110) the gate opens, enabling packages to be vertically transferred, or dumped via a gravitational straight drop from the smart bin through the open gate onto the zoned collector conveyor disposed below the smart bin (S112). The zoned collector conveyor may comprise a plurality of dynamically established accumulation zones or windows.

Packages within an accumulation zone or window of a the zoned collector conveyor may be grouped and tracked together as they are transported as a group, for example down the collector conveyor (S114). The collector conveyor can be configured and positioned to feed onto, for example, a cleated conveyor with the cleats bounding each package group zone or window (S116). Packages from each package group zone or window can then be diverted or transferred for further processing as a group.

Upon a determination to deposit packages into a container fill chute (S118—YES), a package group can be diverted from the cleated conveyor into a hopper, and from the hopper, into a bag fill chute (S120). A bag can then be filled with the packages of the package group, and the package group is logically linked with the bag (S122). Upon a determination that a particular bag is full, or that the fill chute should not otherwise receive more packages (S118—NO), an additional incoming package group can be transferred to another collector conveyor in zones or windows within which the package group continues to be tracked (S140). The package group can then be diverted from such other one or more collector conveyors to another container fill chute (S138). A bag can then be filled with that package group and logically linked with the bag (S122).

After a bag is filled with a package group, and the package group is logically linked with the bag, the bag can be transported to a processing area (S124) where the bag is closed (S126). For example a bag can be zipped closed, and a shipping label can be applied to the bag (S128). Of course, zipping a bag closed is only one example, and the bag or other container can be closed in any of various other ways. A labeled bag containing the package group, can then be transferred for further processing (S130). Additionally, the package group associated with the bag may be cleared, i.e. the package group and bag may be disassociated from a container, for example automatically or via a container release button, and the empty container can be returned (S132), for example to a racetrack, and reloaded with one or more bags, for example to wait in a queue to be filled (S134), and transported to queue area at container fill chute for processing (S122) of another package group.

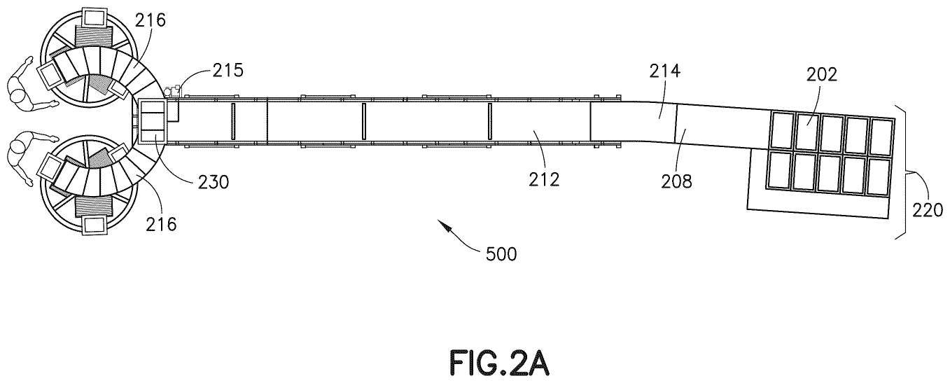

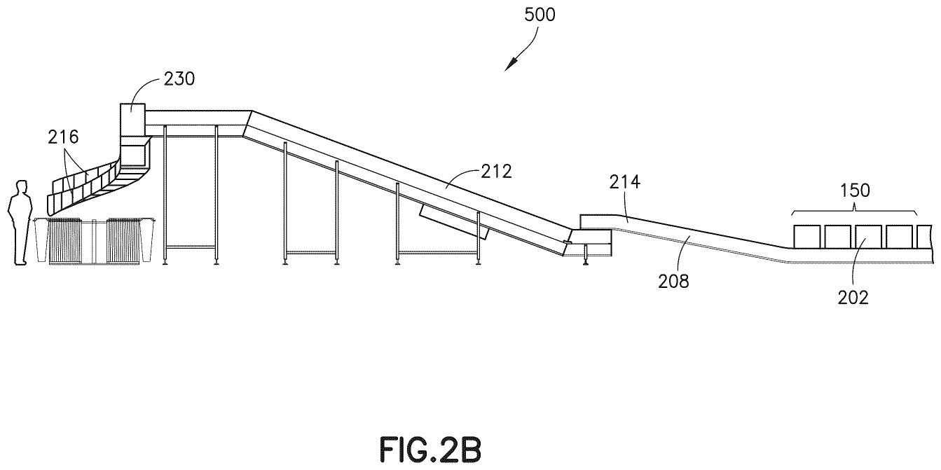

FIGS. 2A and 2B illustrate a top view and a side view, respectively, of an automated sortation system 500 according to an example embodiment. As described herein, a bin according to one or more example embodiments may be referred to, for ease of explanation and without any limitation, as a “smart bin.”. Such a smart bin 202 can be configured to feed a collector conveyor 208, for example located under each side of the sorter 220, for example a sorter having sections. The smart bins 202 may be positioned within the integration section 150 to receive packages from the sorter 220 and to selectively release one or more packages (not shown) onto the collector conveyor 208. As discussed above with respect to FIG. 1, the packages may be released based on any of various criteria and/or a signal from a sensor, such as an optical sensor.

The released packages form a package group, released for example directly below a chute of smart bin 202 onto the collector conveyor 208. Each package group can then be logically tracked down the collector conveyor 208 in zones or windows of suitable dimensions. For example, a zone or window may be a section of the collector conveyor 208 having a width of the collector conveyor 208 and extending about 10 ft in length. The specific configurations of each of a smart bin 202 and collector conveyor 208 can be optimized for efficiency and accuracy of package processing. This includes, without limitation, parameters such as, but not limited to, size, relative positions (vertically and/or horizontally) with respect to each other, and relative displacement (for example due to speed and/or direction of the conveyor 208). For example, for an essentially vertical drop of packages (e.g., due to gravity) from a smart bin 202 onto a conveyor 208, a spread of packages on the collector conveyor 208 and, for example, a the size or dimensions of a zone or window, can be optimized by taking into account one or more of the height or distance from the chute of the smart bin 202 to surface of the collector conveyor 208, the relative moving speed of the surface of the collector conveyor 208 with respect to the smart bin 202, and the relative moving direction of the surface of the collector conveyor 208 with respect to the smart bin 202. In addition, a speed of opening and/or a type of opening (such sliding, hinged, etc.) of a chute or opening of the smart bin 202 can be selectively implemented to facilitate deposition of packages onto the collector conveyor 208. For example, a system can comprise a belt running at 150 fpm, and for such belt speed, a 10 foot windows can be defined in accordance with an example implementation. One or more of a texture, a material, a resilience, and a roughness of the surface or portions of the surface of the collector conveyor 208 can be selected to facilitate the deposition of packages on the collector conveyor 208 and/or the maintenance of packages on the collector conveyor 208. Any combination of any or all of the above-noted parameters, features, and structures can be selectively adjusted and/or optimized to facilitate group tracking and/or processing of packages in accordance with one or more example embodiments described herein.

According to further example implementations, zones or windows can be created or defined on the collector conveyor 208 and/or the cleated conveyor 212 using an encoder pulse width from an optional encoder 215, such that when a certain selected or predetermined number of pulses of the encoder are detected that correspond to a determine window size, a unique token may be created. The unique token can be tracked along the collector conveyor 208 and/or the cleated conveyor 212 using the encoder pulse. This sequence can be repeated for each zone or window. When a package group is released from a smart bin into a zone or window, a smart bin number of the smart bin can be associated with the unique token. When the window reaches the charge of the hopper 230, the system will drop the load into the available hopper 230. At this point the smart bin number is passed to the host system to initiate the printing of a label to be associated with that group of packages.

As shown in FIGS. 2A and 2B, this example system 500 comprises the collector conveyor 208 which transfers packages to a conveyor 212. The collector conveyor 208 may include a rising section 214 in which the collector conveyor 208 is at an inclined angle, with an upper end adjacent to a conveyor 212. The conveyor 212 may be a cleated conveyor, and cleats may form boundaries for one or more zones or windows along the conveyor 212.

From the conveyor 212, package groups can be fed into one of one or more chutes 216 for filling into a respective closable bag 218. Each package group ID may be logically linked to the corresponding bag 218, and a label may be printed and placed on the bag 218. The example system 500 may also include a diverting hopper 230 which receives packages from the conveyor 212 and diverts the packages into a chute 216,

Regarding the cleated conveyor 212, according to an example implementation, a speed of the cleated conveyor can be dynamically adjusted so that tracking zones or windows (for example, ten-foot windows) align with the physical cleat spacing. Such dynamic adjustment can be accomplished using a sensor to detect each cleat and using an encoder.

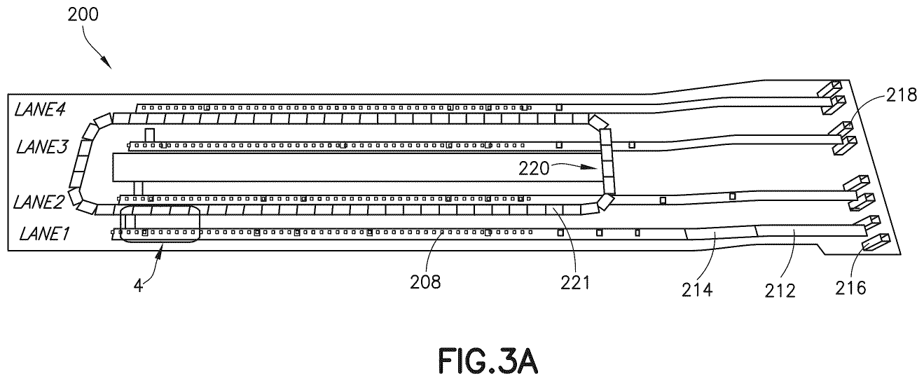

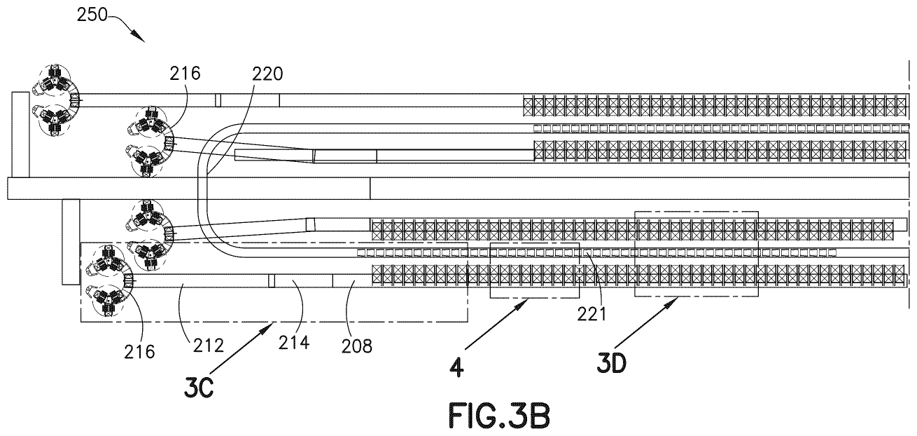

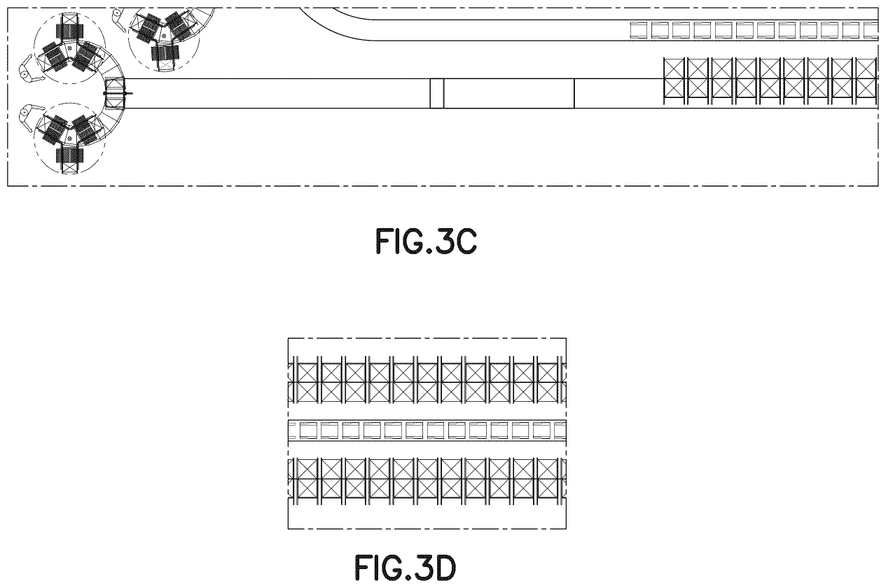

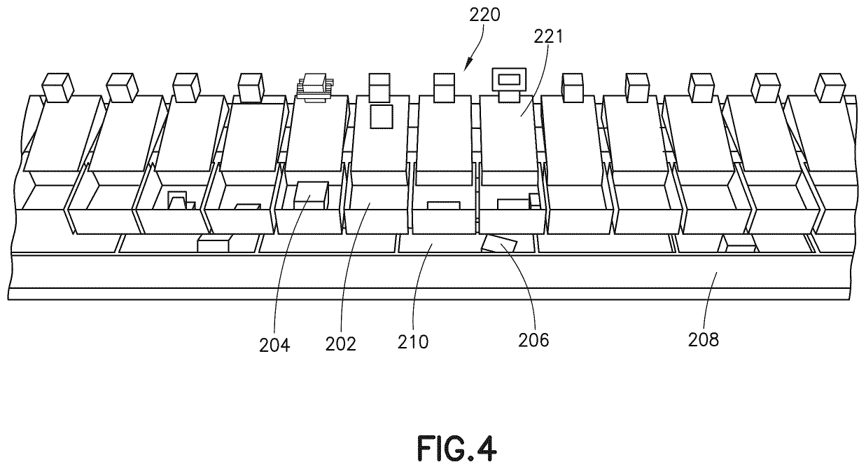

FIG. 3A is a perspective view of an automated sortation system 200 according to another example embodiment. FIG. 3B is a schematic illustration of an automated sortation system 250 according to yet another example embodiment. FIGS. 3C and 3D are enlarged top and side views of sections B and C of FIG. 3B. FIG. 4 is an enlarged perspective view of section A of FIG. 3A and FIG. 3B. References numbers used in FIGS. 3A, 3B, and 4 are the same as those used with respect to FIGS. 2A and 2B, with respect to illustration of analogous elements. In contrast to the example embodiment of FIGS. 2A and 2B, FIGS. 3A, 3B, and 4 illustrate example sections 221 of the sorter 220, and illustrate example packages 206 within the systems 200 and 250.

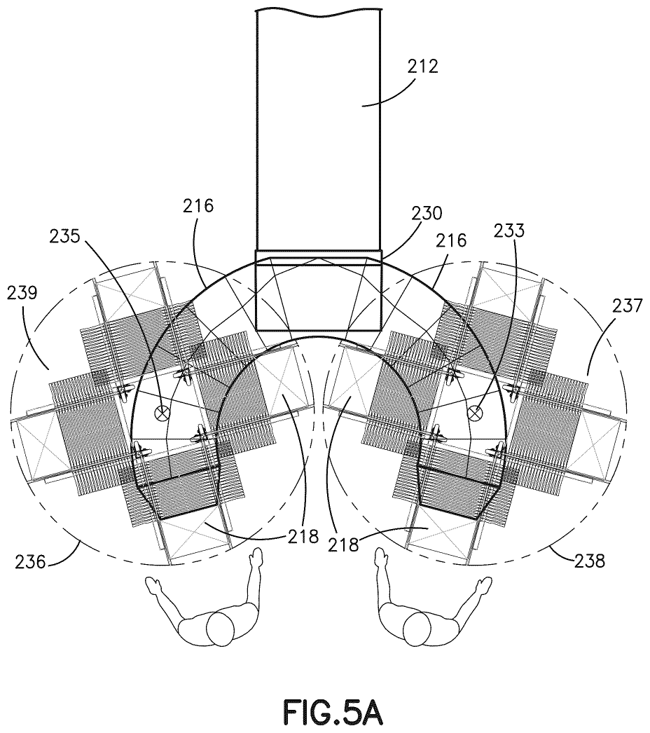

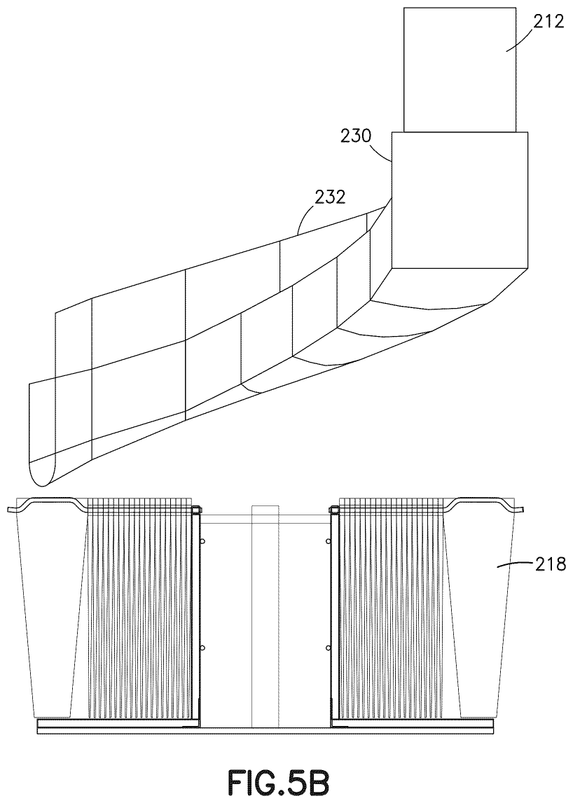

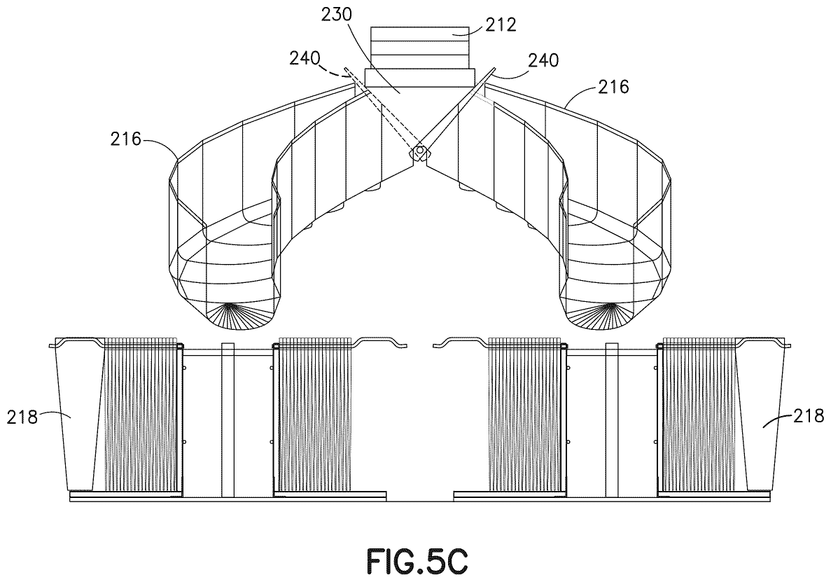

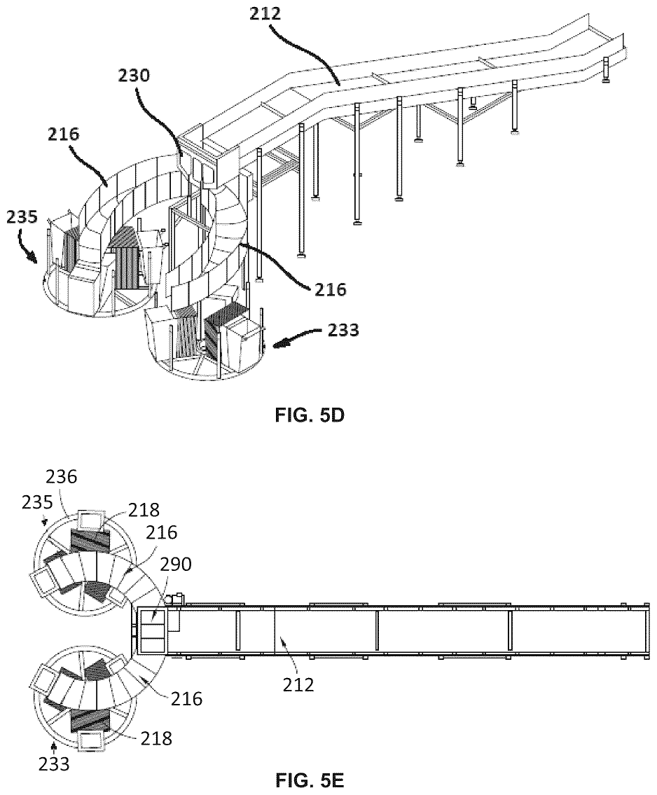

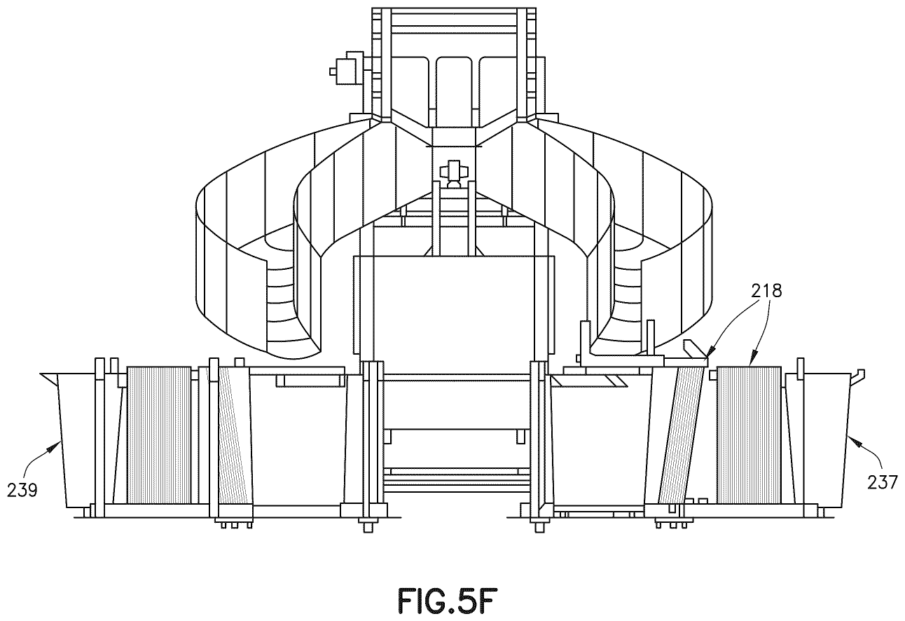

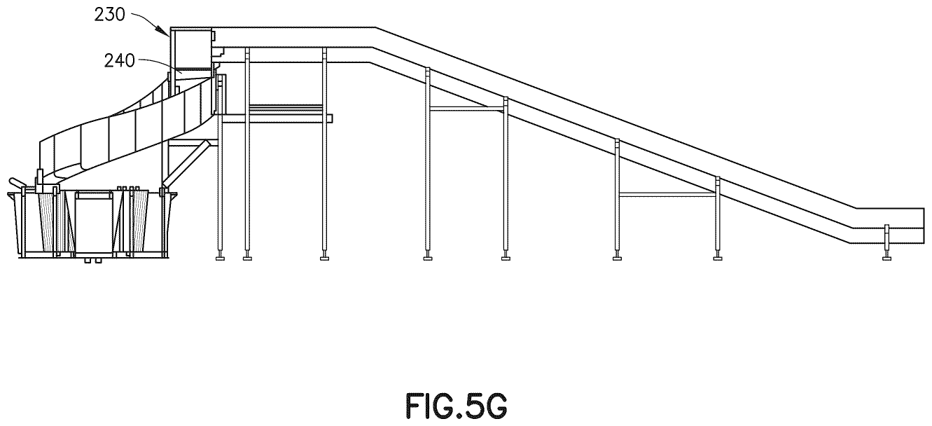

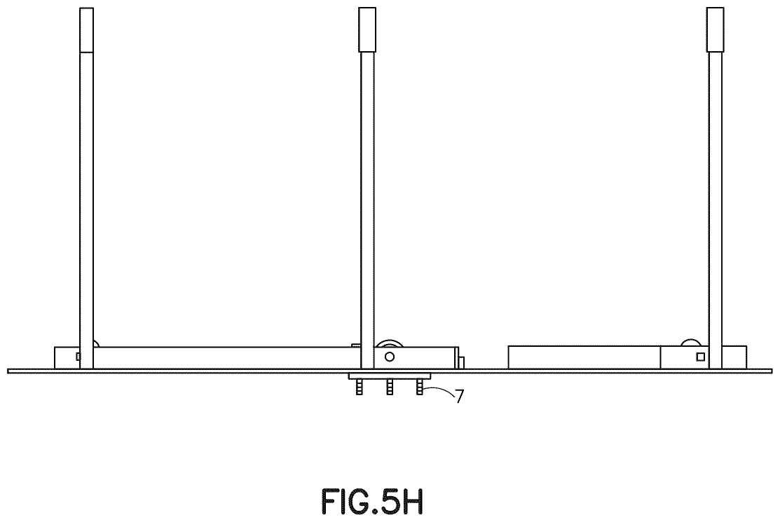

FIGS. 5A-5C illustrate an enlarged schematic view, a side view, and a back view, respectively, of an end of the cleated conveyor 212, the hopper 230, and the chutes 216 of an example system 500, 200, or 250. FIGS. 5D-5G are a perspective view, a top view, an end view, and a side view, respectively, of the cleated conveyor 212, the hopper 230, the chutes 216, and carousels of an example system 500, 200, or 250. As shown, the hopper 230 may be a diverting hopper 230 configured to receive packages from the conveyor 212 and direct the packages of each package group into one of the two chutes 216. FIGS. 5H-5J are a side view, a perspective view, and a bottom view of an example carousel configuration. An example carousel is shown in FIGS. 5H-5J in which, for example: 1 is a formed angle stop; 2 is a center foot weldment; 3 is a flange bearing mount plate; 4 is a 4″ easy turn caster; 5 is a two bolt flange bearing for 2″ shaft diameter; 6 is a grip for 1⅜″ OD; 7 is a ⅜″ anchor bolt; 8 is a steel hex head shoulder screw, ½″ shoulder diameter, 1¾″ shoulder length, ⅜″-16 thread; 9 is a thin hex nut, nylon insert, ⅝″-11; 10 is a medium-strength steel nylon-insert locknut, grade 5, zinc-plated, ⅜″-16 thread size; 11 is a hexagon socket button head cap screw ¼″-20×½″ LG; 12 is a hexagon socket head cap screw ⅝″-11×1.75″ LG; 13 is a hexagon socket button head cap screw 5/16″-18×½″ LG; 14 is a ¼ plain washer (inch) type A and B; 15 is a ⅝″ plain washer (inch) type A and B; and 16 is a carousel weldment.

The diverting hopper 230 may comprise one or more flip gates 240 which selectively block or permit a package to flow into one of the chutes 216. Alternately, the diverting hopper 230 may comprise air-operated, rodless cylinders mounted at 90° with respect to each other and at 45° with respect to a floor, such that the cylinders can be selectively retracted or extended to open and close a flip gate 240 which selectively blocks or permits a package to be diverted into one of the chutes 216. Chutes 216 can be configured to deposit packages of a single package group into an open bag 218, such that an operator may then close the bag, apply a label, remove the bag, and open a subsequent bag.

According to an example implementation, closable bags can be arranged at each of the chutes 216 in merry-go-round configurations 236 and 238, such that, at each of the merry-go-round configurations 236 and 238, a bag 218 can be staged on each one of a plurality of carts, for example four carts, such as carts 237 or 239, below a respective chute 216. The bags 218 can be mounted on a carousel 233, 235 which can be rotated to present a staged empty bag, as needed. Alternately, a linear shuttling system may be used in which shuttling carts with empty bags are moved from left to right, or a single cart can be in position and replaced once empty bags are all used. Once filled, a closable bag 218 can be labeled, closed, and placed onto a return conveyor to go back into the parcel sorting system.

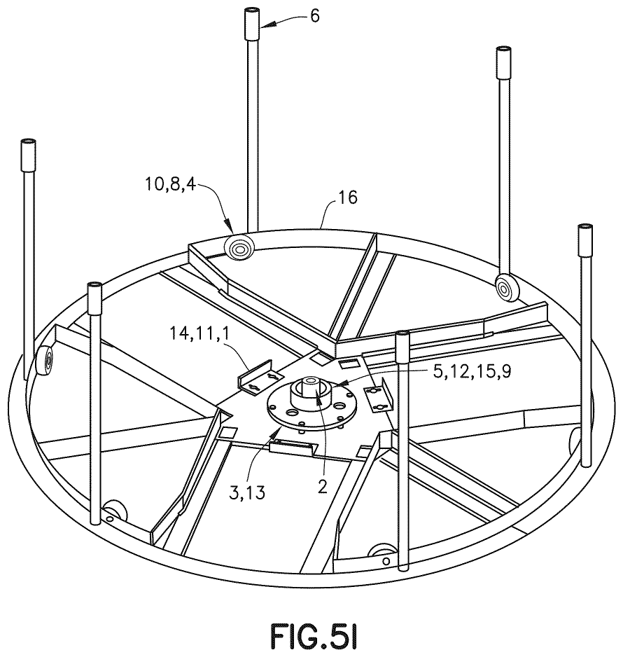

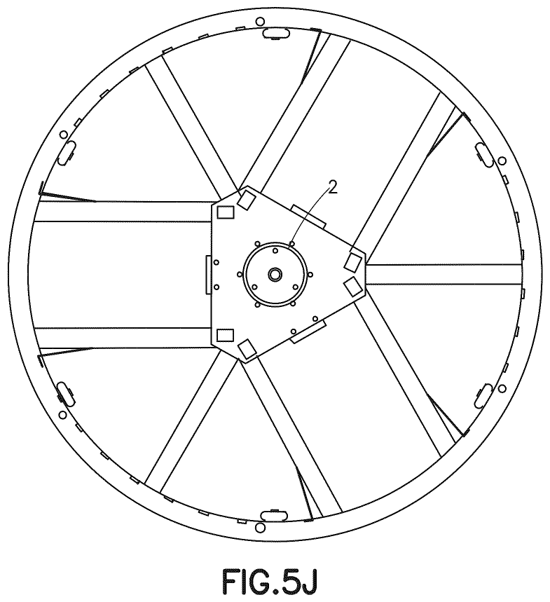

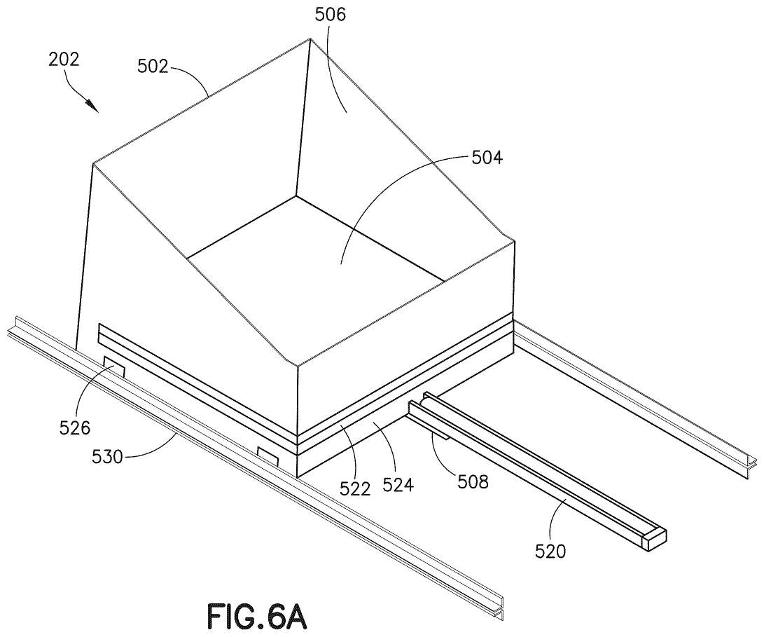

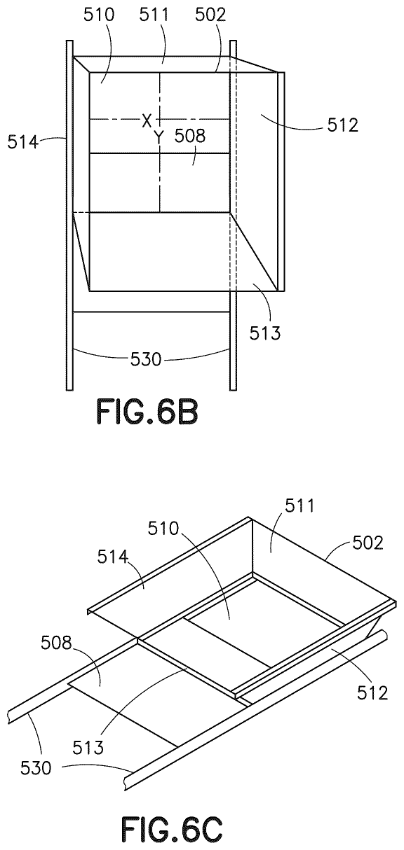

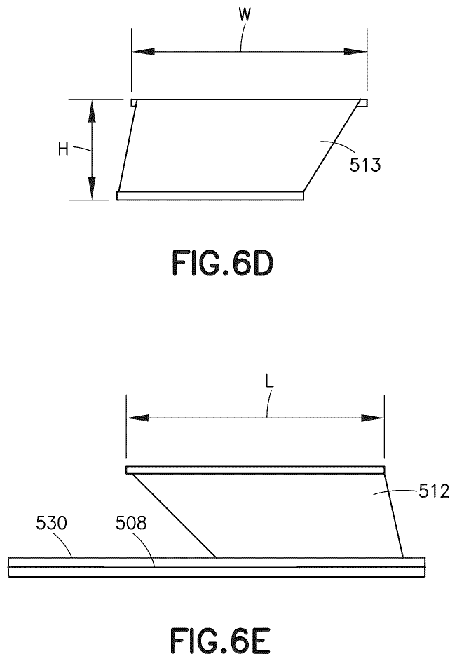

FIGS. 6A-6E illustrate a smart bin 202, according to an example embodiment: FIGS. 6A, 5B, and 6C are different perspective views of the smart bin; FIG. 6D is a plan view of the front of the smart bin; and FIG. 6E is a plan view of a side of the smart bin. As shown in FIGS. 6A-6E, the smart bin 202 includes a bottom 504, and a side wall 502 extending upward from each edge of the bottom 504, such that the bottom 504 and side wall 502 together define a cavity 506, therewithin. The cavity 506 can have any of a variety of dimensions, including cross-section X-Y, height H, width W, and length L, sufficient to accommodate a plurality of packages 204 that can be transferred or released to zones or windows 210 of collector conveyor 208. The bottom 504 comprises a gate 508, which is moveable between an open position and a closed position. When the In the gate 508 is in the open position, a passage 510 is defined by lower edges of the side wall 502, and a rear edge of the bottom 504/gate 508, as shown in FIGS. 5B and 6C. The passage 510, thus open, enables packages 204 to be released from the cavity 506, for example onto a collector conveyor 208. The side wall 502 may comprise four adjoining sections 511, 512, 513, and 514, which together to define sides of the cavity 504 having an essentially rectangular bottom with a cross-section X-Y. One or more of the sections 511, 512, 513, and 514 may slant outward from their respective bottom edges, such that one or more of the sections 511, 512, 513, and 514 may have a rectangular shape or a trapezoidal shape. The cavity 504 may have a height H, a width W, and a length L, as shown in FIGS. 6D and 6E. The gate 508 can be a slide gate having a linear guide system 530 and a linear actuator 520. The guide system 530 may include two or more guide rails, as shown in FIGS. 6A, 6B, 6C, and 6E. Further components may be included in the smart bin 202, such as a strip bush holder 522, a strip bush 524, and a pillow block 526, which may facilitate operation of the smart bin 202.

According to example implementations, Ultra High Molecular Weight Polyethylene (UHMW), or other types of low-friction material, can be used for manufacturing and/or for lining of chutes, hoppers, bins and other wear surfaces.

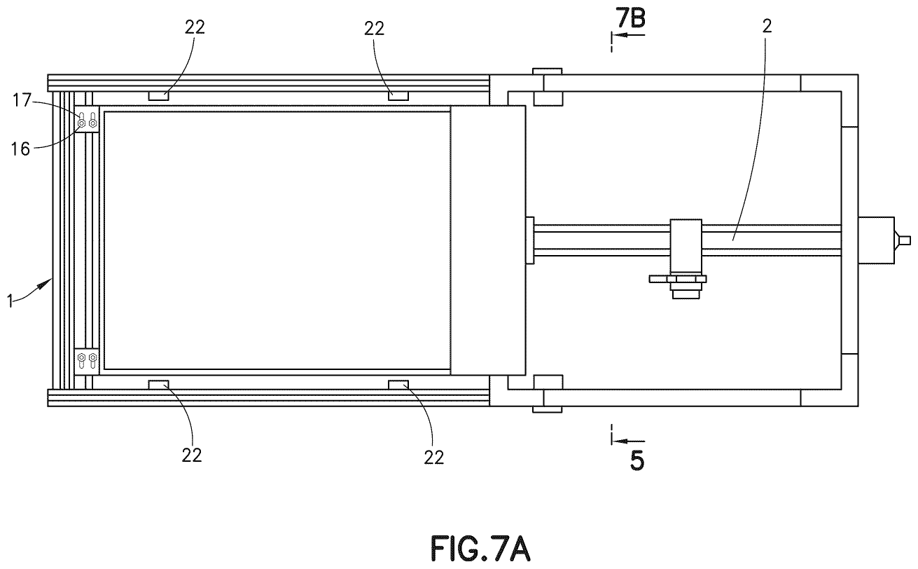

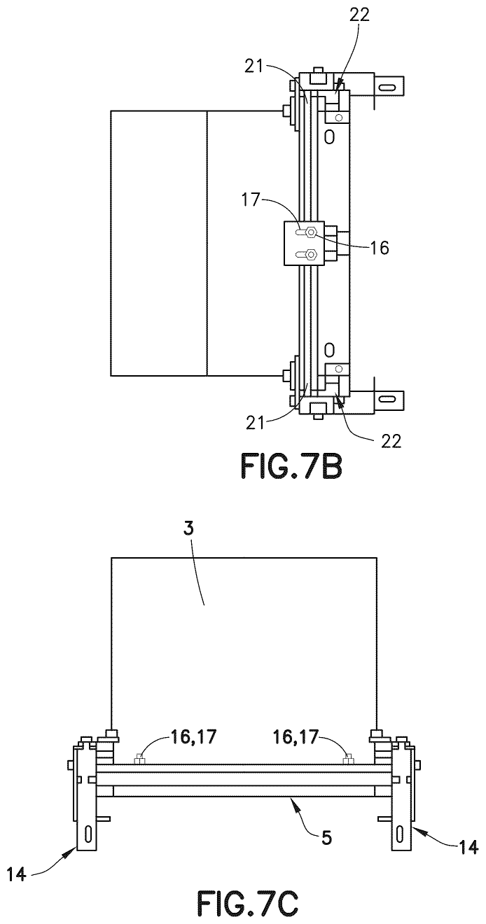

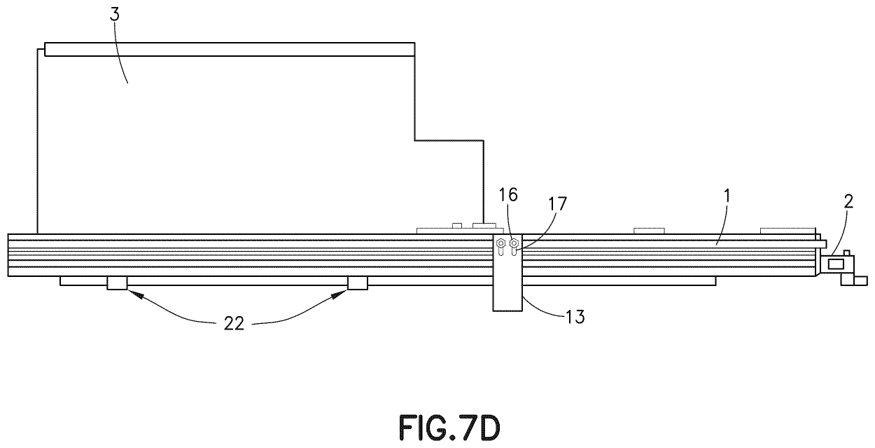

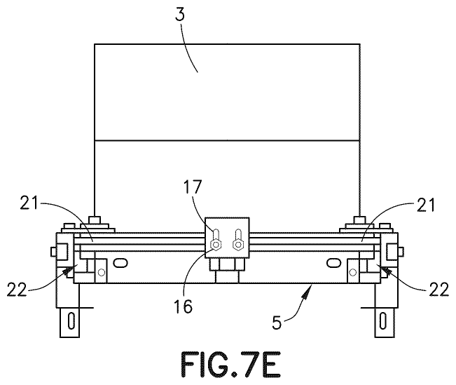

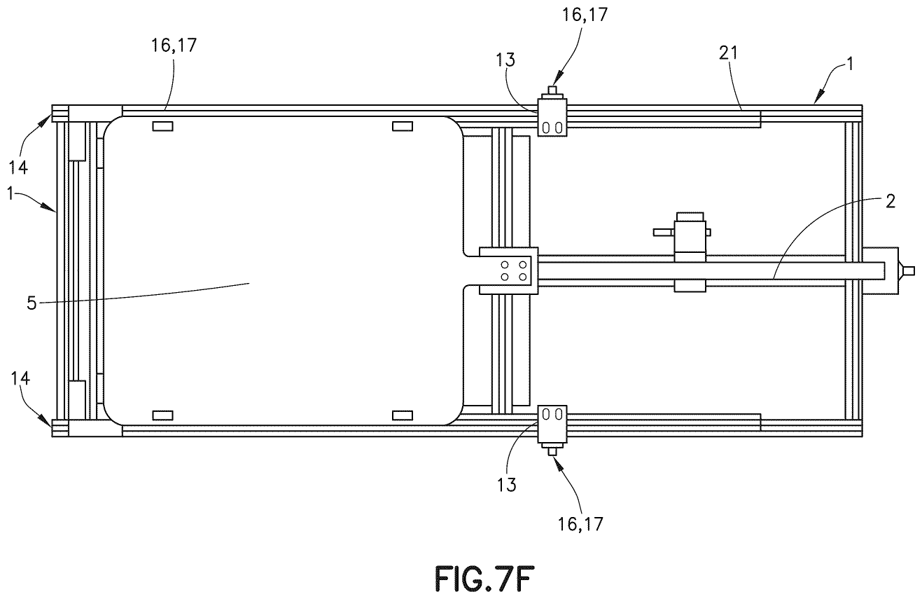

FIG. 7 illustrates another example smart bin 602 according to an example implementation in which: 1 is a frame assembly; 2 is a cylinder assembly; 3 is a smart bin assembly; 4 is a frame connector plate; 5 is a slide gate; 6 is a smart bin-right; 7 is a smart bin-left; 8 is a smart bin-sorter side; 9 is a smart bin-operator side; 10 is a smart bin-belt retainer plate; 11 is a smart bin-belt wiper; 12 is a smart bin-belt return plate with weldnuts; 13 is a slide gate mounting angle; 14 is a slide gate end mount plate; 15 is a smart bin-UIHMW wiper; 16 is a t-bolts for extrusion slots; 17 is a t-bolt lock nuts for extrusion slots; 18 is a side rails extrusion×69.5 long (1×3 slots); 19 is an intermediate strut extrusion×25.375 long (1×1 slots); 20 is an intermediate struts extrusion×25.375 long (1×3 slots); 21 is a bearing rail igus size 16 rail×1346 mm long; and 22 is a bearing igus drilling with bearings size 16.

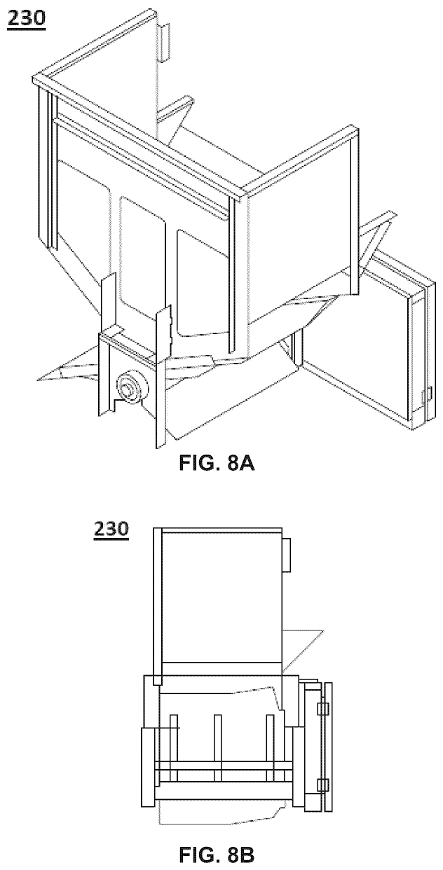

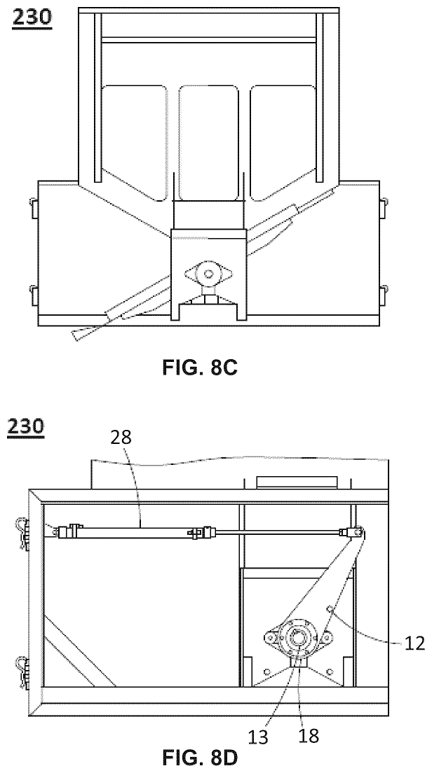

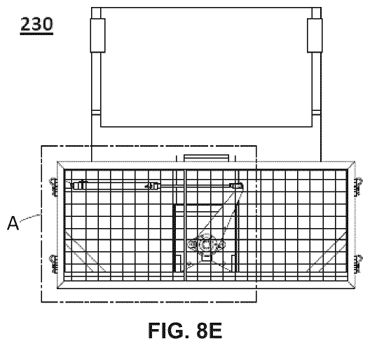

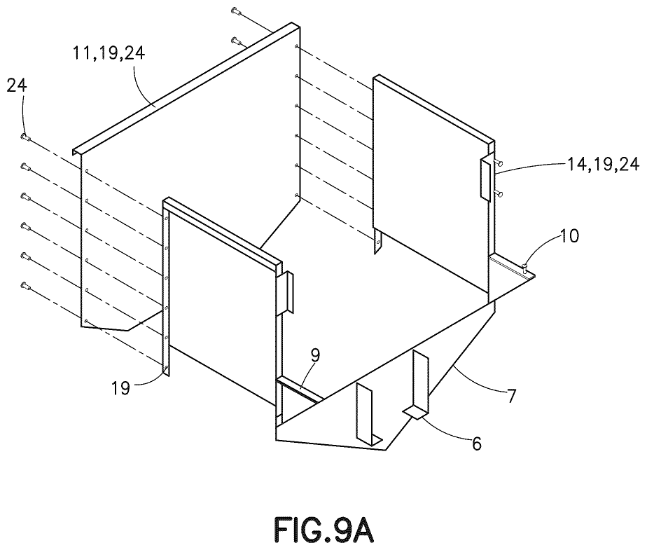

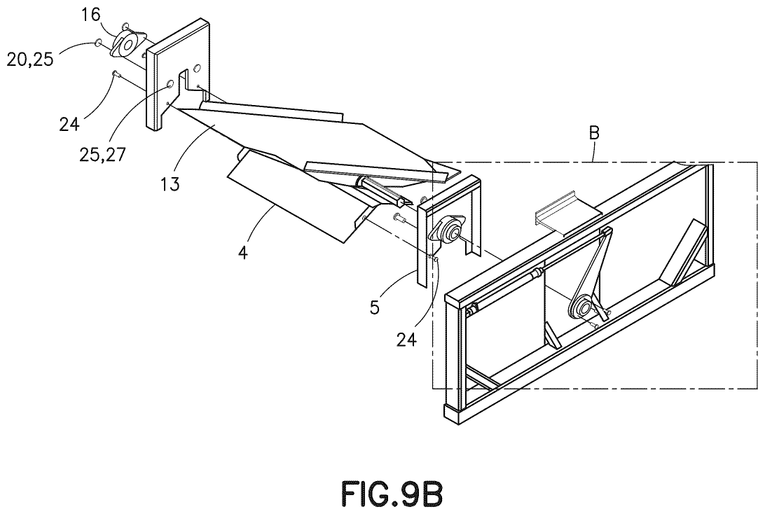

FIGS. 8A, 8B, 8C, 8D, and 8E illustrate a perspective view, a side view, a front view, a detail view of portion A of FIG. 8E, and a back view, respectively, of a hopper 230 according to an example implementation; while FIGS. 9A, 9B, and 9C illustrate an exploded view, an exploded view with the guard hidden, and a detail B, respectively, of an example hopper, in which: 1 is a ACB-02 temporary ship weldment; 2 is a cylinder weldment; 3 is a front panel weldment; 4 is a lower center support; 5 is a flange bearing mount bracket; 6 is a support angle; 7 is a rear steel panel; 8 is a side panel; 9 is a triangle bracket 2; 10 is a triangle bracket; 11 is a polycarbonate panel; 12 is an actuator plate; 13 is a gate weldment; 14 is an angle to cleated conveyor; 15 is a cylinder guard; 16 is a 2-hold flange bearing and clamp for 1.5″ diameter shaft; 17 is a bronze brushing—5.0 ID×8.0 OD×8 mm LG; 18 is a flanged shaft clamp for 1.5″ diameter shaft with keyway; 19 is a hex nut, nylock, 5/16—18; 20 is a hex nut-½″—13; 21 is a hex nut—¼″—20; 22 is a ¼—20 UNC—2¼ HS HCS; 23 is a ¼—20 UNC—⅞, HS HCS; 24 is a 5/16-18×⅞, HSBHCSI25 is a ½″ washer; 26 is a ¼″ washer; 27 is a ½—13 UNC—1.5, HBI; 28 is a pneumatic cylinder-25 mm bore×305 mm (12″) stroke; and 29 is a shock absorber with ¾—16 UNF thread-body.

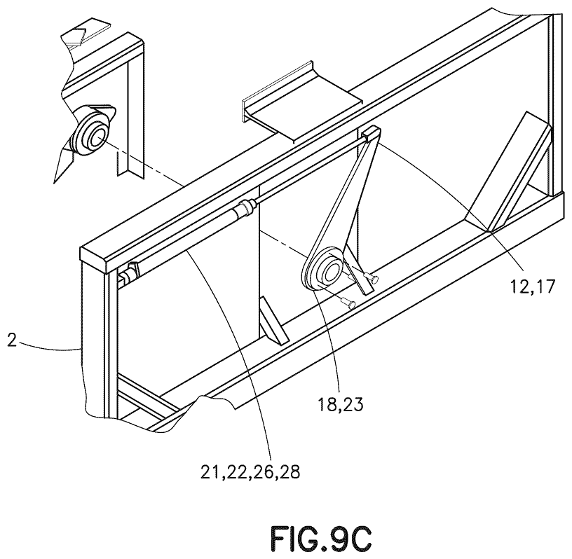

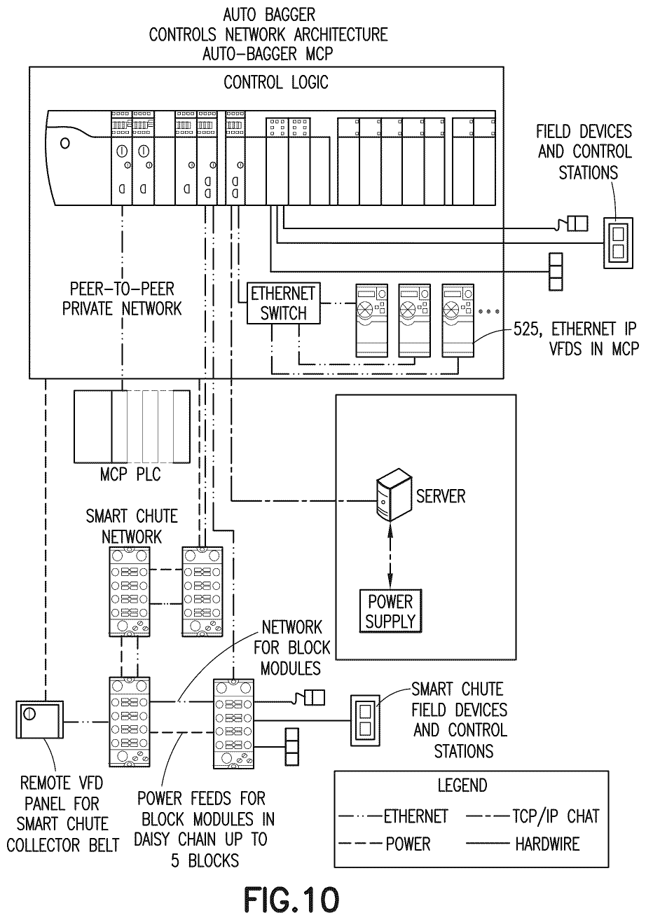

According to an example embodiment, a turnkey solution may include the provision and installation of conveyor systems, as described herein, as well as all motors and control devices. FIG. 10 is a schematic illustration of a main control panel according to an example embodiment. Such a main control panel can be installed to control an automatic sortation system, such any of those as described herein. The main control panel may include a plurality of input/output (I/O) modules, field devices, and implement variable frequency drive (VFD) technology. One or more main control panels may be networked together and may control coordinated operations. If a new main control panel is networked with an existing main control panel in an existing system, Human Machine Interface (HMI) and programmable logic controller (PLC) program development can be performed for the new main control panel and modifications, if any, may be made to programs in the existing main control panel to accommodate any new system elements.

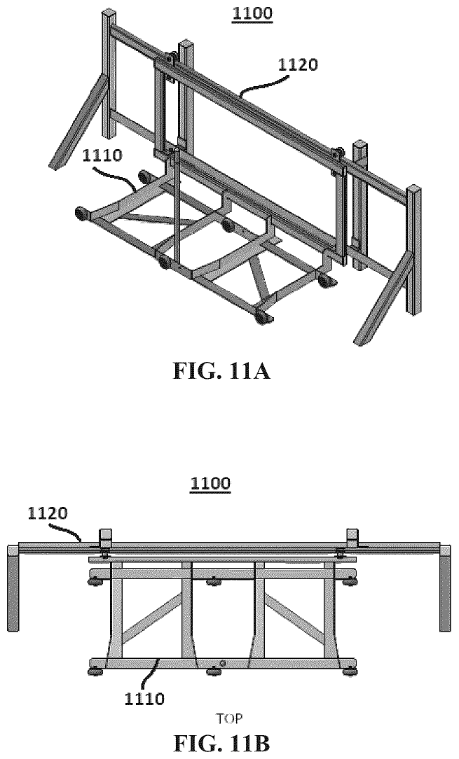

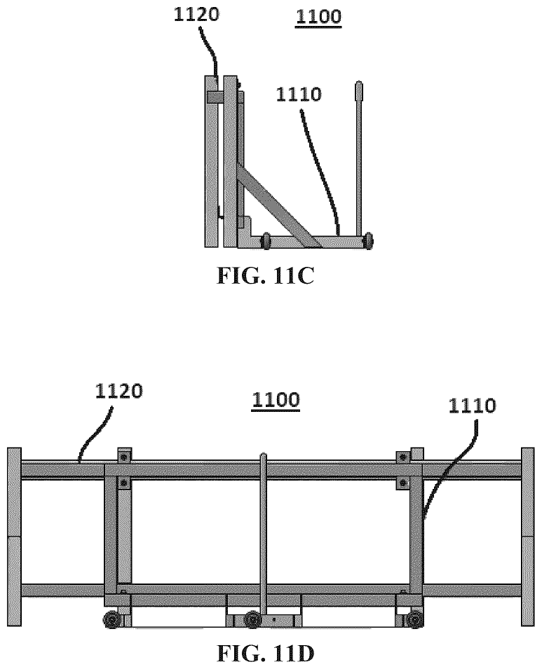

According to an example embodiment, a two-way linear rail assembly can be provided as a replacement for merry-go-round configurations 236 and 238, such that, for example, a two-way linear rail assembly can be installed at each or any of the merry-go-round configurations 236 and 238 and/or carousel 233, 235 shown in an example of FIGS. 5A-5I. FIGS. 11A-11D illustrate an example implementation of a two-way linear rail assembly 1100 comprising a support 1120 and a carriage 1110.

In an example implementation, one or more bag carts, such as for example two surepost bag carts (TSK-03), can be loaded into respective position of carriage 1110 and located underneath a hopper, such as hopper 230. When a bag is full the operator can remove the bag, and for example take it to a collector conveyor. When the bag cart is empty of bags, carriage 1110 can be rolled over so that a bag cart full of empty bags can be placed underneath the hopper and the bags continue to be filled. While an operator is filling bag at a current position another operator can remove an empty bag cart and replace it with a cart full of bags.

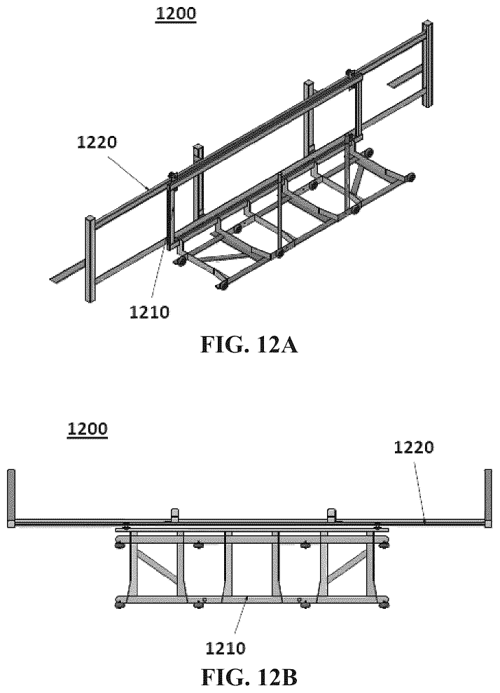

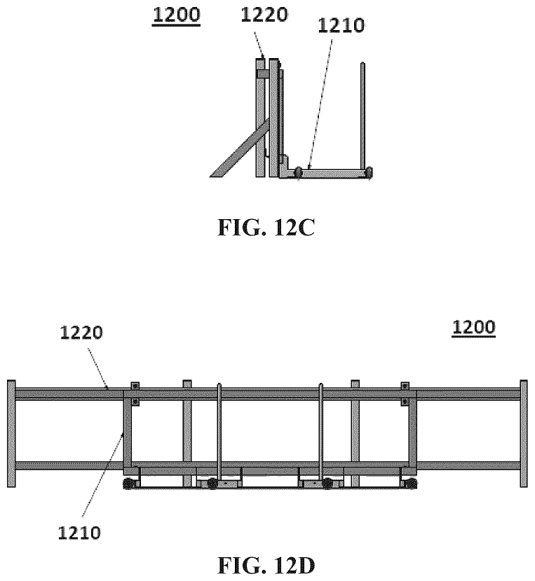

According to an example embodiment, a three-way linear rail assembly can be provided as a replacement for merry-go-round configurations 236 and 238, such that, for example, a two-way or a three-way linear rail assembly can be installed at each or any of the merry-go-round configurations 236 and 238 and/or carousel 233, 235 shown in an example of FIGS. 5A-5I. FIGS. 12A-12D illustrate an example implementation of a three-way linear rail assembly 1200 comprising a support 1220 and a carriage 1210.

In an example implementation, one or more bag carts, such as for example three surepost bag carts (TSK-03), can be loaded into respective position of carriage 1210 and located underneath a hopper, such as hopper 230. When a bag is full the operator can remove the bag, and for example and take it to a collector conveyor. When the bag cart is empty of bags, carriage 1210 can be rolled over so that a bag cart full of empty bags can be placed underneath the hopper and the bags continue to be filled. While an operator is filling bag at a current position another operator can remove an empty bag cart and replace it with a cart full of bags.

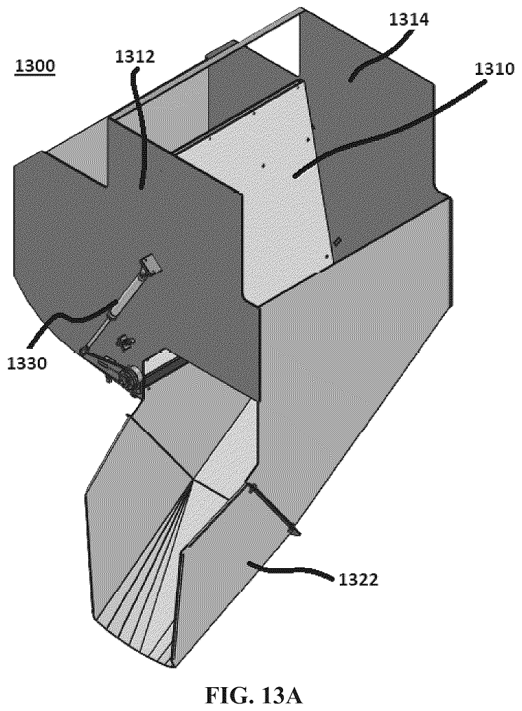

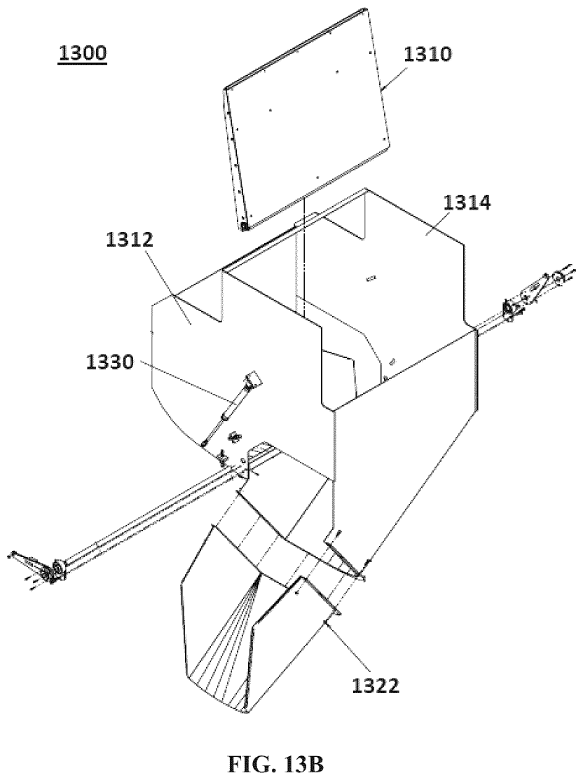

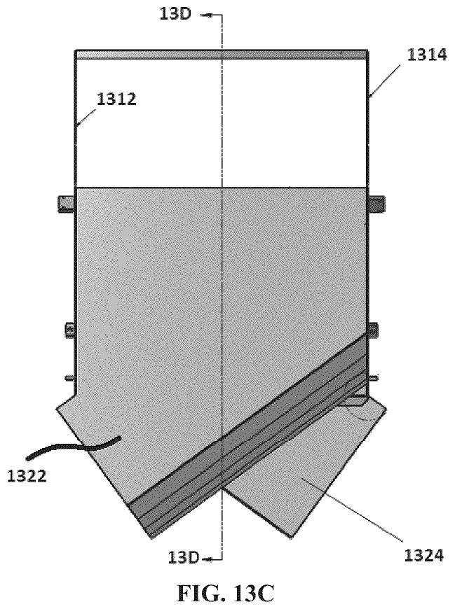

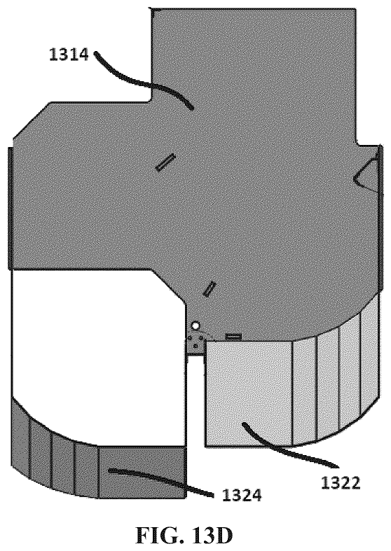

Referring to FIGS. 13A-13D, in an example embodiment, a hopper 1300 can be used in place of hopper 230, for example when using a linear rail assembly, such as assembly 1100 or 1200, for two loading positions, which can be referenced as positions A and B. In an example implementations, a three-position linear rail assembly, such as assembly 1200, can be placed under both sides of the chutes. A cleated conveyor assembly, such as assembly 212, can be configured to feed packages between side plates 1312 and 1314 of hopper 1300, and based on a predetermined or desired destination of a slug of packages, a gate assembly 1310 can be turned, for example to 90 degrees, if packages need to go to chute 1322, or for example to 45 degrees if packages need to go to chute 1324. In an example implementation, gate 1310 can be actuated by a pneumatic cylinder 1330 which can be configured on one side, or both sides, of the hopper assembly 1300. Hopper assembly 1300 can be used for example when less packages per hour are needed, and less space is available since it can be implemented to have two positions to feed two linear rail assemblies, such as assemblies 1100 and/or 1200.

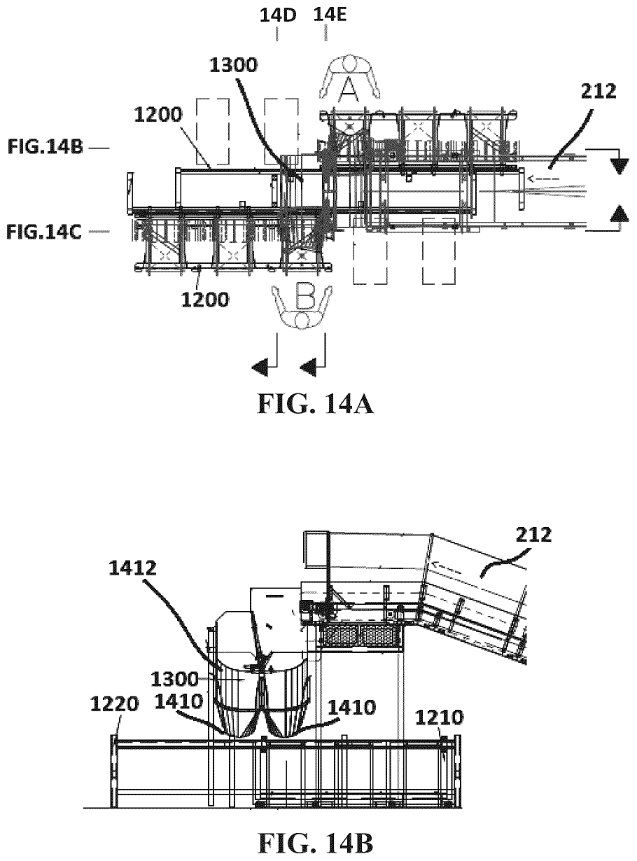

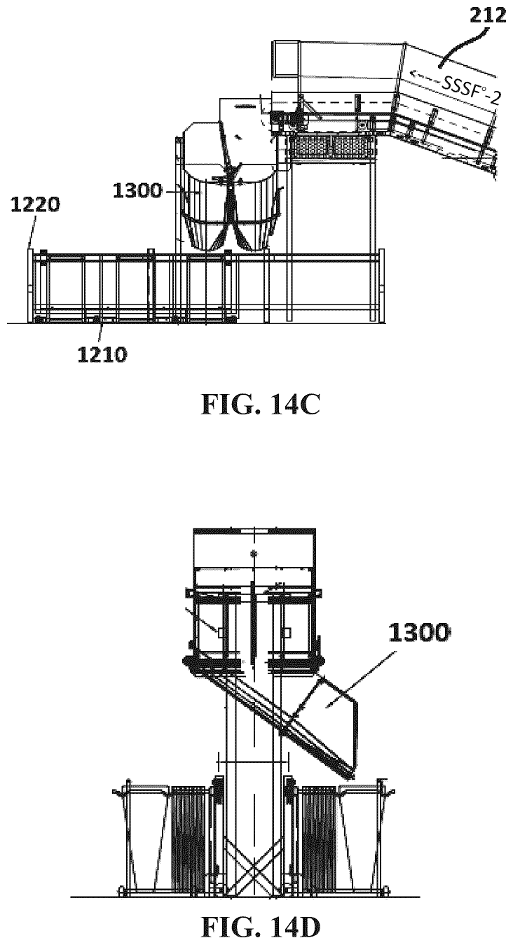

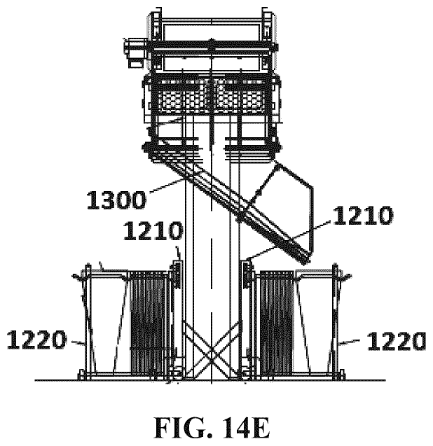

FIGS. 14A-14E illustrate in an enlarged schematic plan view and various cross sectional views, respectively, example embodiment of a system, such as system 500, 200, or 250, implementing hopper 1300 and linear rail assemblies 1100 and/or 1200. Example implementations shown in FIGS. 14A-14E illustrate how a hopper 1300 integrates with a cleated conveyor 212 and three-way linear rail assemblies 1200. As illustrated in FIG. 14A, packages feed from a cleated conveyor 212 to either side to each operator A and B. In an example implementation, a flip gate 1410 can be provided on the end of a chute of hopper 1300 that the operator flips up or down to hold each bag in place while packages are dropping into them. In yet further example implementation, a hopper curtain 1412 can be provided to slow down packages as they fall down the chute of hopper 1300.

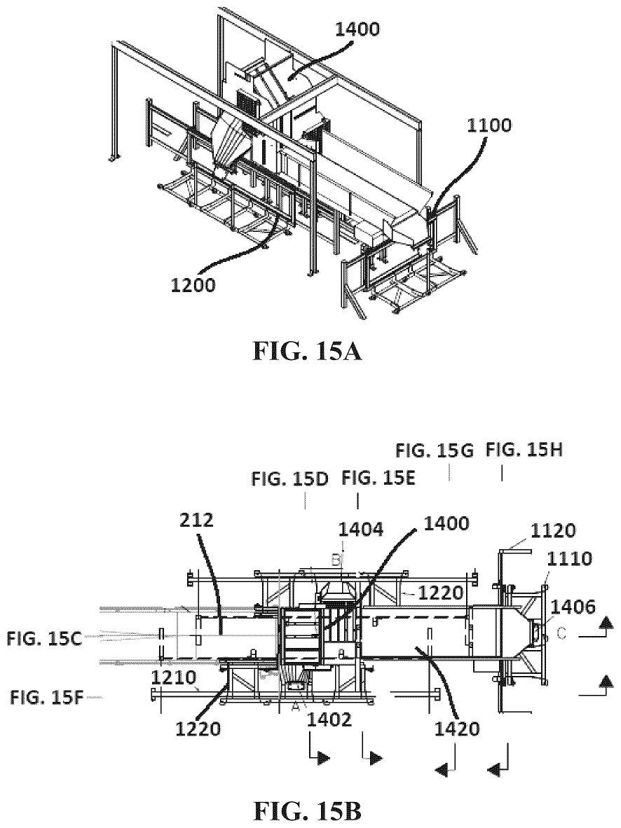

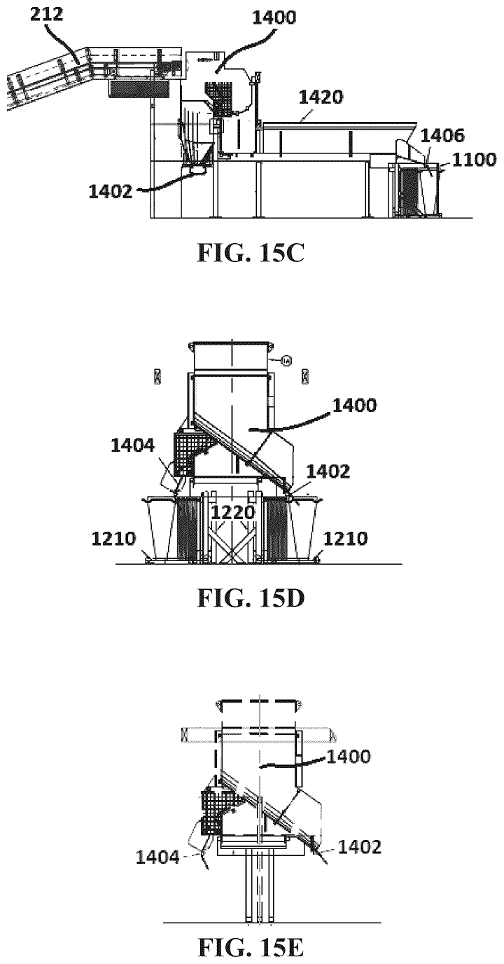

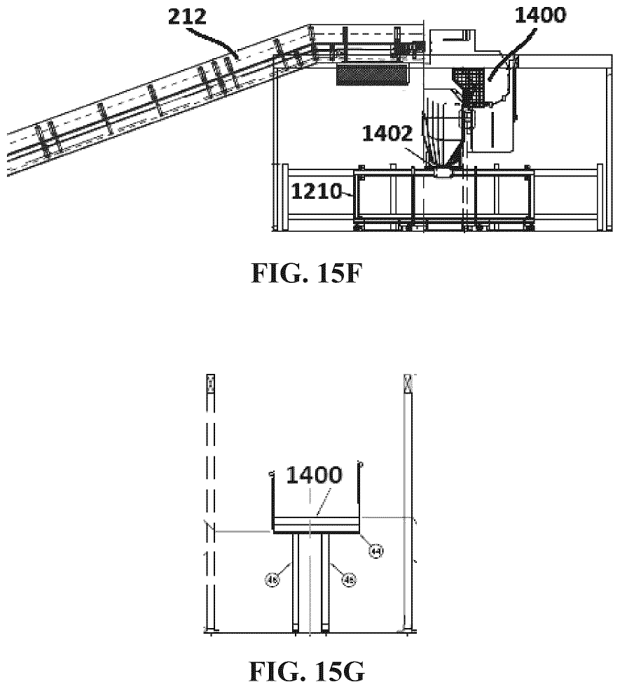

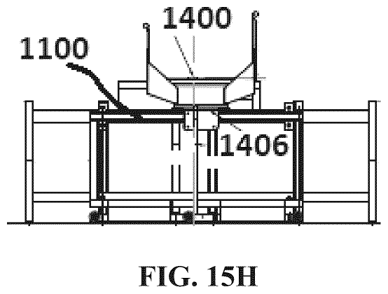

FIGS. 15A-15H illustrate in an isometric view, enlarged schematic plan view and various cross sectional views, respectively, another example embodiment of a system, such as system 500, 200, or 250, implementing hopper 1400 and linear rail assemblies 1100 and/or 1200. Example implementations shown in FIGS. 15A-15H illustrate how a hopper 1400 integrates with the cleated conveyor 212 and the two-way linear assemblies and three-way linear assemblies. In an example implementation, hopper 1400 has similar function to that of hopper 1300; however, in addition to the two positions A and B of hopper 1300, hopper 1400 implements an added third position, C, 1406. According to an example implementation, a first gate assembly 1402 of hopper 1400 flips up or down to either feed the A position or B position, and a secondary gate 1404 can implemented at the B position that flips up or down to continue down the B position or feed onto a conveyor that takes the slug of packages on a belted conveyor 1420 to the C position. In a further example implementation, A and B positions can be configure to feed a three-way linear rail assembly 1200 and the C position feed a two-way linear rail assembly 1100. An example configuration of FIGS. 15A-15H can be used when, for example, more packages per hour are needed and/or the floor space is sufficient.

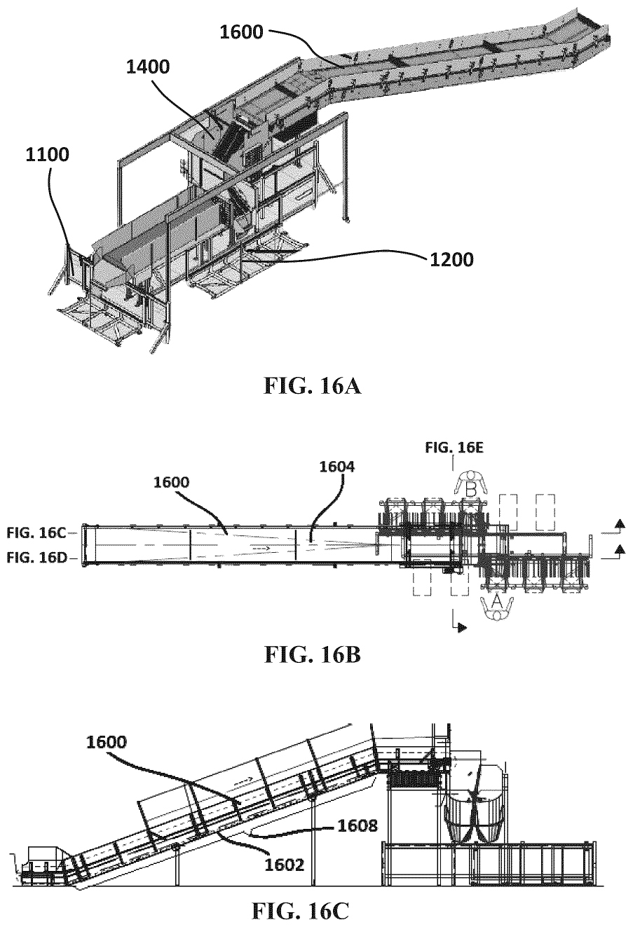

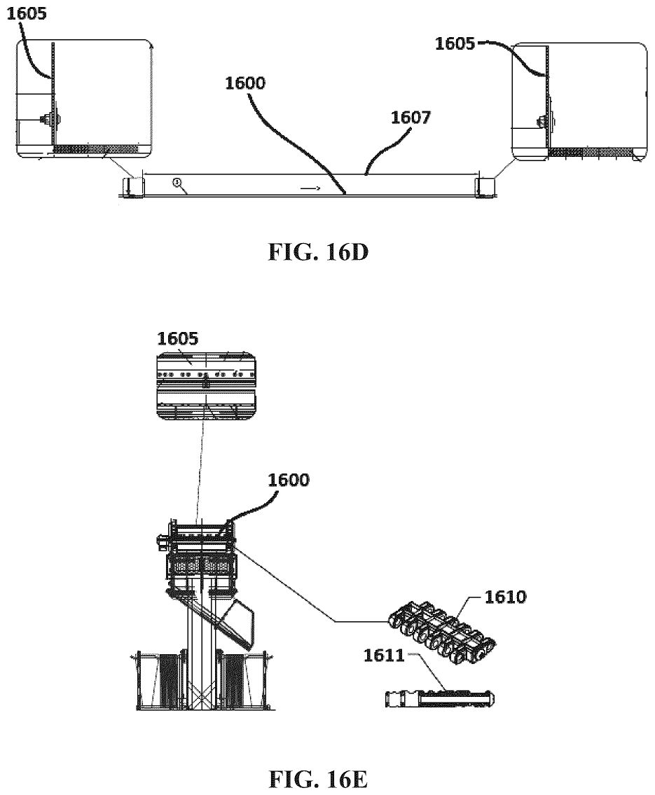

FIGS. 16A-16E illustrate in an isometric view, enlarged schematic plan view and various cross section and enlarged views, respectively, another example embodiment of a system, such as system 500, 200, or 250, implementing cleated conveyor 1600. Example implementations shown in FIGS. 16A-16E illustrate how cleated conveyor 1600 can be configured with hopper 1400 and linear rail assemblies 1100 and/or 1200.

More generally, FIGS. 16A-16E illustrated an example of the cleated conveyor assembly 1600 that can feed either hopper 1300 or hopper 1400, and the processing using conveyor 1600 can also be performed as in the example of FIGS. 5D 5E 5F and 5G. FIG. 16A example shows cleated conveyor 1600 and the hopper 1400 along with three-way linear rail assembly 1200 and two-way linear rail assembly 1100.

Referring further to FIGS. 16A-16E, details of example implementations can include custom belting 1604 that takes slugs of packages up an incline 1602 in section 1608. In an example implementation, belting 1604 can include cleats 1605 configured at intervals 1607 on belting 1604. In an example implementation, incline 1602 can be about 20 degrees from horizontal.

In yet further example implementations, custom flags (for example, metal) can be embedded in the belting to trigger a proximity sensor 1610 and keep the belting tracked so that the location of essentially each slug of packages is the conveyor 1600 can be determined. In an example implementation, proximity sensors 1610 can be installed on both sides of belting 1604, for example on an outside edge thereof. In a further example implementation outside sprockets 1611 on head and tail are installed so that they will not interfere with the proximity blocks.

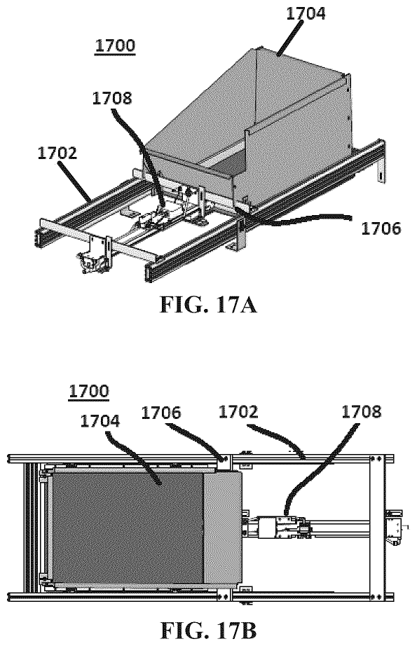

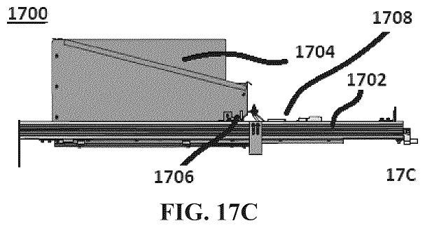

FIGS. 17A-17C illustrate in an isometric view, top view and side view, respectively, another example embodiment of a smart bin 1700 that can be deployed in any one of systems 500, 200, or 250, implementing cleated conveyor 212 or 1600, and any one of hopper configurations such as hopper 230, hopper 1300, or hopper 140, and either a carousel or linear rail assembly.

In an example implementation, smart bin 1700 can have at least the functionality similar to that of a smart bin 202 described with reference to FIGS. 6A-6E above. According to further example implementations, smart bin 1700 can comprise hold-down tabs 1706 on the front of bin body 1704, so that package weight does not shift the bin for example with respect to frame assembly 1702. According to yet further example implementations, smart bin 1700 can comprise proximity flags and one or more sensor added to for example slide cylinder 1708 for a more accurate readings when the gate is opened or closed.

Another example embodiment, which can be implemented independently of, or complimentary to, an automated sortation system embodying various features described with reference to FIGS. 2A-17C capable of performing the automated sortation method as described with reference to FIG. 1, provides an auto bagging or auto packaging system and methodology.

In an example implementation of an auto bagging system complimentary to the automated sortation, steps S124 and S126 of FIG. 1 are further automated such that: (1) the packages that were logically tracked by the automated sortation system are dropped into a large bag, which can be configured either in a carousel 233,235 or a linear rail system 1100,1200, as described above with reference to FIGS. 2A-17C; (2) the large bag determined to be full of packages is presented for closure (zipping); (3) the bag is automatically closed (zipped), for example by a robotic system with a custom end-effector; and (4) the closed bag exits the system while a new (empty) bag is placed into the rotation to be filled.

In an example implementation, an empty bag can be placed on into the system, for example on a carousel 233, 235 or a linear rail system 1100, 1200, manually. Alternatively, the system can be configured such that multiple bags, for example a couple of hundred bags, can be loaded into the system before beginning operation and an operator could then tend to several such systems at one time to add additional bags as needed.

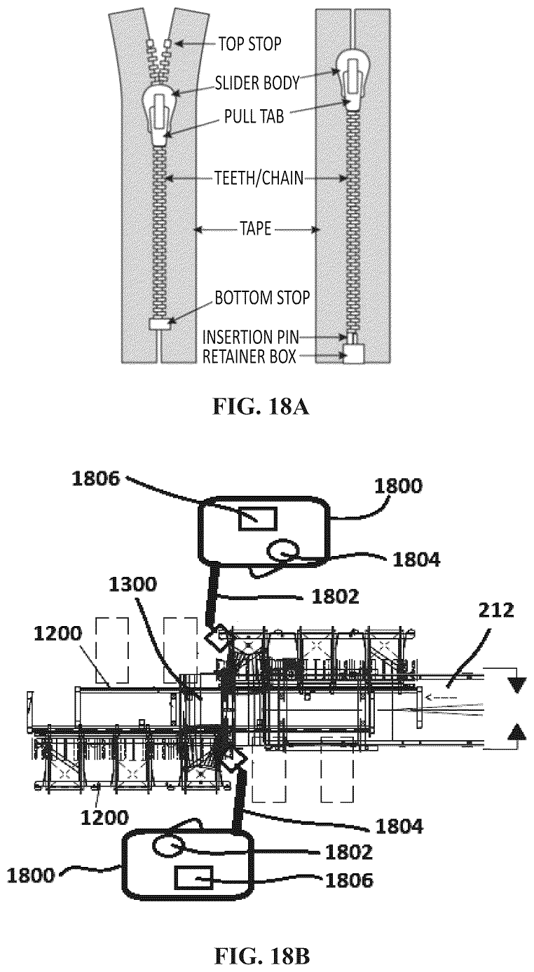

In an example implementation of closure with an automatic zipping component, a robot with a vision system to zip each bag can be deployed. As illustrated in an example of FIG. 18, such a system can include an electromechanical system and components 1800 comprising mechanical devices that are actuated by electricity, including without limitation pneumatics and/or magnetics, such as robotic arm 1802 in communication with one or more sensors 1804 and/or a computer processor 1806. In example embodiments, such system is configure to ensure that smaller items or parcels enter a bag or container, that such bag or container is closed by either zipping the bag or through other means (e.g., zip tie) for example by a robotic arm 1802 controlled by the processor 1806 based on stored and/or communicated information and/or command (including, without limitation, input from sensor 1804), and such bag or container is transported from the filling station to downstream processing.

In an example implementation, such a system 1800 can be capable of executing commands stored or transmitted by wire or wirelessly including specific routines and/or subroutines which execute to control the auto bagging process including the actions and timing of the electromechanical devices to, without limitation, ensure smaller items or parcels enter a bag or container, that such bag or container is closed by either zipping the bag or through other means, and such bag or container can be moved from the filling station to downstream processing.

While the example embodiment of FIG. 18B illustrates an auto bagging system components 1800 deployed to complement an automated sortation system, system 1800 can also be deployed in a stand-alone configuration adapted to automate bag closure in any system where a large quantity of bags or other containers need to be filled and closed.

One or more example embodiments described herein may provide various modes of operation for a conveyor system implementing smart bin technology including, but not limited to: manual or automated release of packages into package groups. A manual release can be based on, for example a visual inspection of the smart bin. A fully automated release can employ any of a variety of hardware and/or software configurations, such as, but not limited to: a proximity sensor, a volume sensor, a weight sensor, a photo sensors, and the like, in order to automate the release based on an output and/or control thereof. One or more of the example embodiments described herein may be used in conjunction with any of a variety of package group tracking techniques, such as using zone and/or window sensors arranged on the conveyor 208 or in proximity thereof. All electronic sensing components can be integrated into an operation monitoring system and/or an automated system such as a learning computer system.

One or more example embodiments provide an electromechanical system comprising a computer processor, a sensor and a robotic arm, wherein when said package group is conveyed for further processing including transferring into a container the plurality of the packages of the package group, said electromechanical system closes the container by the robotic arm controlled by the computer processor based on stored or communicated information or commands based on input from the sensor.

One or more example embodiments provide an electromechanical system wherein the input from the sensor comprises an indication of the container being full to a predetermined level.

One or more example embodiments provide an electromechanical system wherein the container is a bag with a zipper closure.

One or more example embodiments provide an electromechanical system, wherein the robotic arm comprises a plurality of movement axis.

One or more example embodiments provide an electromechanical system, wherein the robotic arm comprises and end effector configure to close the zipper closure.

One or more example embodiments provide an electromechanical system, wherein the electromechanical system further comprises means for position the container to facilitate the closure of the container.

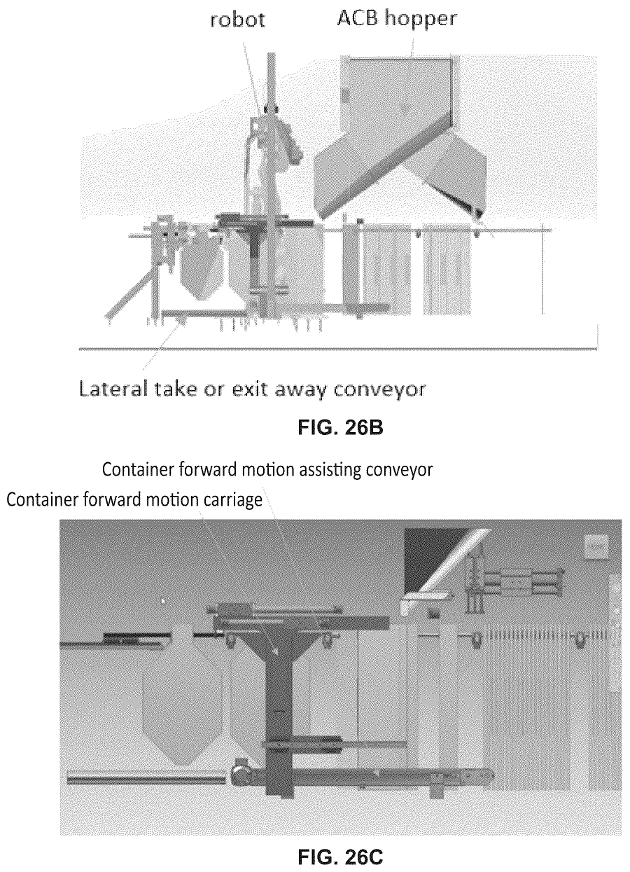

Referring to FIGS. 26A-26D, a system according to an example embodiment may be configured with respect to a package source, such as an ACB hopper disclosed in U.S. Published Patent Application Pub. No. 20230159281, and may include for example a component for supporting flexible containers, for example in container storage groups, a tusk configuration for supporting and transporting individual containers from storage groups to a container filling position, a mechanism for migrating from a tusk to tuskless configuration to facilitate filling of the container, and transitioning from tuskless to tusk configuration after filling to transport container for closing, a mechanism for staging the container for closing, such as a partial close mechanism, a multi-axis tool, such as a zipping tool, for closing the container, and a release mechanism for releasing the container for a tusk support. In another example configuration, a forward motion carriage and motion assisting conveyor can be provided, as shown in FIG. 26C, to facilitate transfer of a container to a fill and/or close and/or release position. In a further example configuration a container take away conveyor, as shown in the example of FIG. 26B, can be provided to receive and transport the released closed containers. In yet further example implementation, a bag isolating gripper, as shown in FIG. 26D can be provided to facilitate acquisition of a single flexible container, such as a bag, from storage groups.

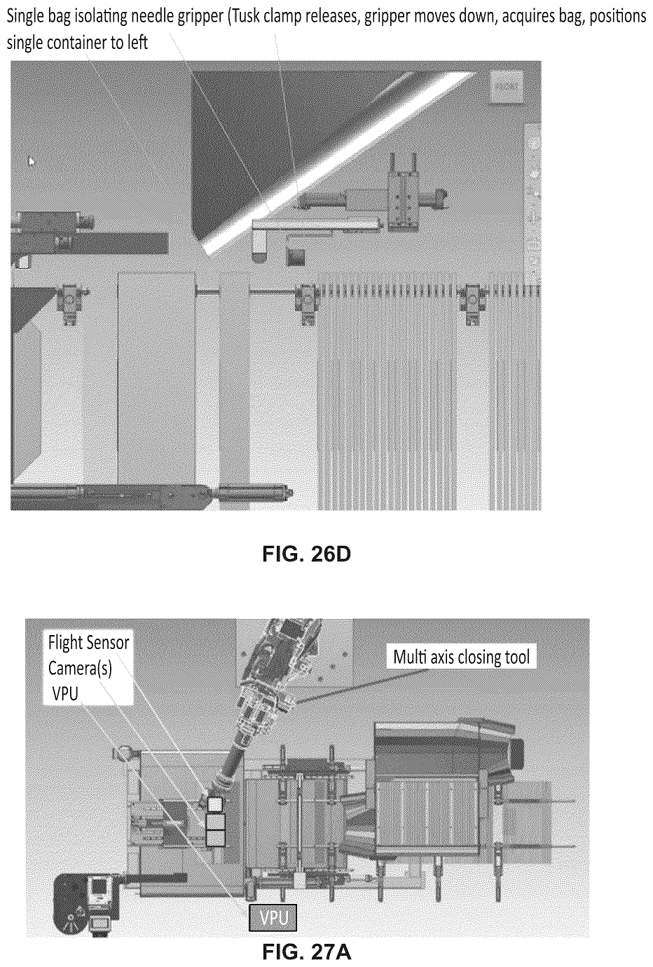

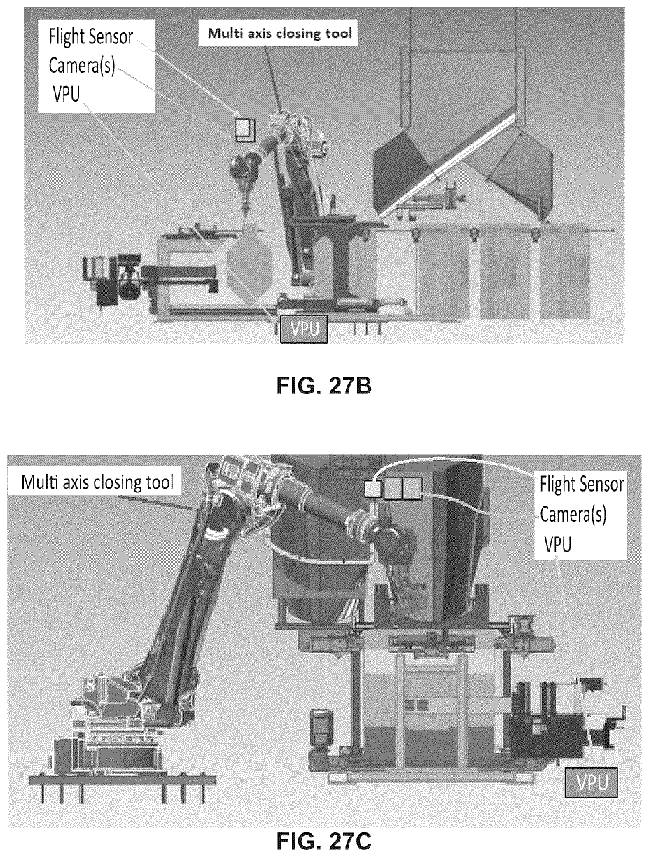

Referring to FIGS. 27A-27C and the labels provided therein, a system according to an example embodiment can be configured comprising a vision processing unit, (VPU), which can identify the specific objects, and their locations, as may be required, for example, to close the container. Such objects include, but are not limited to, the container(s), the closing mechanism(s), container contents, container positioning and holding apparatuses. In addition to the identification of these object(s), the VPU can be configured to extrapolate the position of each object in three-dimensional space. This array of data can be utilized for logical decisions and calculating trajectories using inverse kinematics to guide the multi-axis coordinated motion-controlled device(s), such as the multi axis closing tool, to successfully close the container(s).

In a further example implementation, the VPU logical component can make specific determinations based upon statistical probabilities to communicate the appropriate actions to electro-mechanical device(s). These logical decision algorithms can be at least in part the guidelines directing various subordinate controllers and devices with regards to container closing or zipping. VPU and any other logical component or other components may also utilize artificial intelligence and machine learning to enhance performance over time.

According to yet further example implementation, a biopic vision system can be provided that comprises two or more two-dimensional color sensors and (1) time of flight sensor with a 60-degree by 45-degree lens positioned, for example above the container closing mechanism, as diagrammatically illustrated in FIGS. 10A-10C, to provide the VPU with a required area of view. External illumination may be provided to standardize and equalize ambient lighting conditions. In addition, lens filters may be applied to optimize the lighting to mitigate specular reflection and or filter less desirable light wave frequencies. The analog video signal can be converted to a high-speed transferrable digital version for transmission to VPU. For example, image processing unit can filter the area of view provided in real time by the vision system. The filtering and optimization of the image system can be calibrated during the system calibration stage after installation, prior to being operational. Additional image filtering and optimization can be completed by numerous algorithms in real time to ensure image quality. These include, but are not limited to smoothing, sharpening, and edge enhancements. The image processing unit can apply multiple algorithms including, but not limited to dithering, half toning, Elser difference, feature detection, blind deconvolution, seam carving, and segmentation. A convolutional neural network, CNN, may also be implemented to interpret the spatial relations for more adaptive real-time high-speed convolution and sampling for object detection incorporating, but not limited to, convolution layering, pool layering, fully connected layering, dropout and activation. Supplemental illumination with a 30-degree field of view and polarizer(s) can be used when applicable.

In a still further example implementation, the system can utilize a specifically designed end effector, or tool, to perform the task. In example implementations of the disclosed embodiments, an end effector (or a gripping portion) can be adapted for attachment to, and manipulation of, any portion, or configuration, of any closure mechanism in order to perform an associated closure process, such as for example and without limitation: a zipper car, including without limitation its slider body or a ring or a tag, for containers with a zipper closure; a zip-tie, or any portion thereof, for containers with a zip-tie closure; and other.

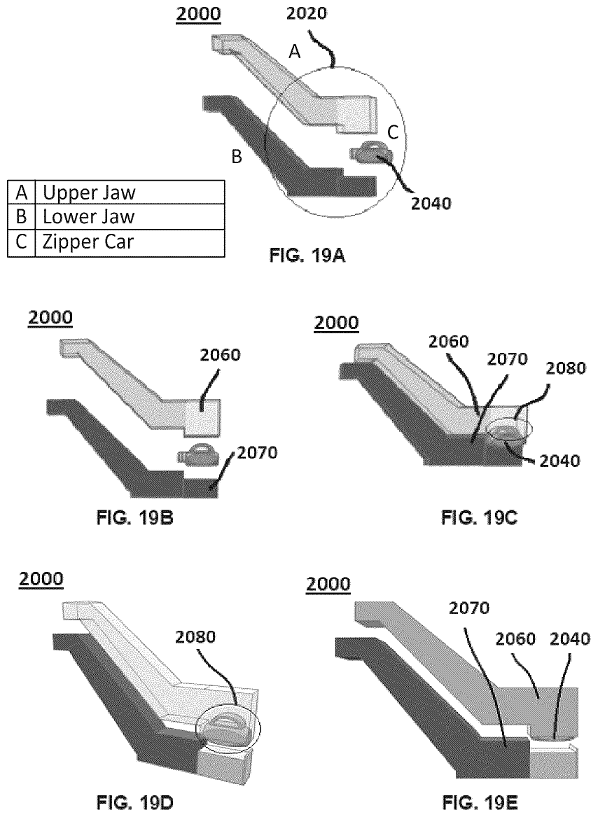

Referring to conceptual diagrams of FIGS. 19A-19E, for containers requiring a zipping process, example implementation of end effector 2000 includes a gripping portion 2020 designed to acquire a slider body 2040 of a zipper. In a further example implementation, gripping portion 2020 can comprise a clamping jaw 2060/2070, which in a still further example implementation can include a portion 2080 contoured with zipper slider body profile cutouts, for example to facilitate a more robust means to obtain the slider body quickly and securely, as illustrated in FIGS. 19C-19E. According to example implementations, attachment to the slider body by a ring, or pull tab used in conventional zipping scenarios, while possible is not needed, such that in a certain example implementation of a tool 2000, attachments to the zipper car, such as rings or tags, will not interfere with gripping the slider body 2040 itself.

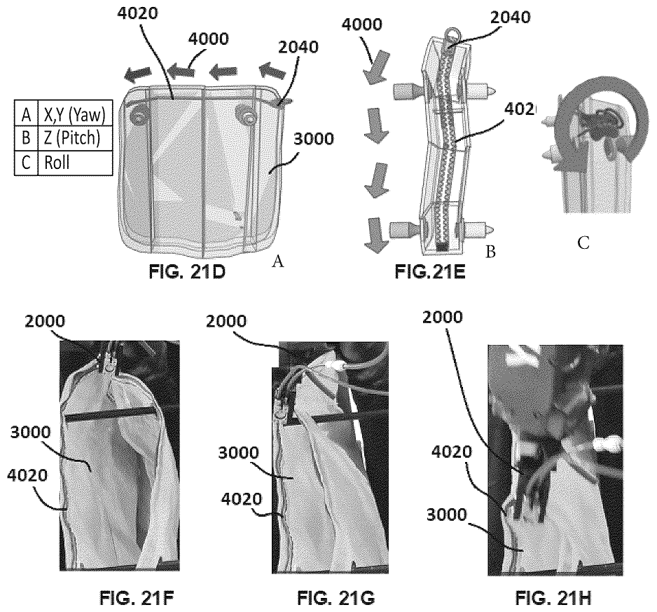

According to a further example implementation, tool 2000 can be configured for easy insertion into a container to be zipped to acquire slider body 2040 and then to be easily extracted from the container upon successfully zipping the container. The design also facilitates gripping during a contoured or three-dimensional zipping motion pathway required for the zipping of certain containers, as described below with reference to FIGS. 21A-21H. The end effector may also incorporate a dynamic force feedback system to be utilized by the VPU for any real time motion pathway adjustments required. In addition, the VPU can be configured to determine the pitch, yaw and roll of the slider body 2040 to facilitate end effector alignment during zipper slider body 2040 acquisition.

According to an example embodiment, a primary function of a zipping motion determination includes VPU calculated trajectories or motion pathways. The VPU, upon inspection of the container, can determine the optimal kinematic approach calculated to reduce any counter forces exerted on the zipper slider body 2040 and subsequent end effector 2000 encountered along the zipping pathway thus increasing its effectiveness. In addition, small lateral, longitudinal, and vertical motions can be dynamically applied as necessary to keep the zipper slider body 2040 in motion should any zipper car/zipper teeth or element resistance be encountered.

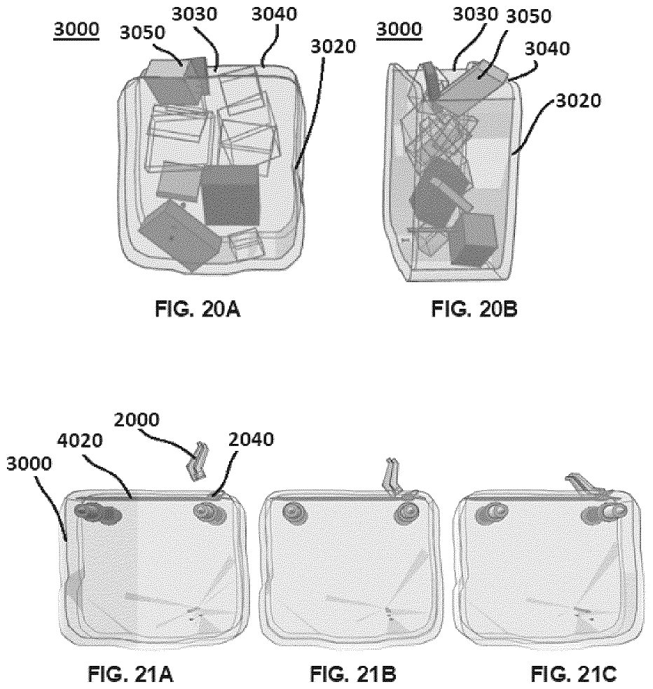

In yet other example implementations, for certain containers, such container 3000 diagrammatically illustrated in FIGS. 20A and 20B requiring zipping to close, an automated clamping function can be provided to secure the containers to provide stability during image acquisition, zipper slider body acquisition and zipping motion. Such clamps can be positioned with respect to, for example 5 cm to 10 cm below, the zipper teeth or elements. The clamps can be configured to provide both enough tension to smooth the zipper pathway and force to hold the container in place during the zipping process. Once the zipping has been validated by the VPU, the clamps can be released for the next stage in the process.

According to an example embodiment, a closing operation cab be performed as follows:
Step 1, Closing inspection, is the process by which a determination is made to attempt to automatically close the container. If a positive outcome is derived from the corresponding image analysis, then the system is ready to make closing preparations. If a negative outcome is derived, the VPU may call upon any system to remediate the perceived concern and after confirmation of the remediation attempt, the VPU can re-inspect for closing. If remediation is not possible, then the VPU can notify subsystem(s) of the alarm or fault condition to be rectified.
   The apparatus can notify the VPU that a new container has been transported into place. Upon notification the VPU can begin analyzing the current images to determine, for example, the following:
      Has a container object been detected for closing
      What is the type of container
      What is the position of the container
      What is the condition of the container
   Many of these steps and processes can be multi-threaded to be handled in parallel by the VPU to facilitate increased performance. Upon container object identification and closing mechanism object detection by the VPU object detection module, a closing mechanism analysis can occurs as follows:
      What type is the closing mechanism
         Zipper
         Zip-tie
         Velcro
         Stitch
         Clamped
         Magnetic
         Compression
         Spring
         Folded
         Other
      If a zipper object is detected, the (VPU) can analyze the condition of the closing mechanism to determine if zipping is possible. For example, the zipper could be missing the zipper slider body or numerous zipper teeth, ‘elements’, rendering the zipper non-functional. The zipper slider body can be analyzed to determine if there is an attachment such as a ring, pull tab or other device to assist in the zipping of the container. The position of the zipper slider body can also be analyzed to determine if the zipper is in the open, closed, or in an intermediate position. If the zipper slider body is not in the appropriate position, measures can be taken to move the slider body to the fully open position. The image analysis can also provide the x, y, z coordinates or (pitch, yaw, roll) for the appropriate kinematics positioning of the end effector. This can be important for precise positioning of the end effector to securely obtain the zipper car. This includes the insertion trajectory for the end-effector to be positioned inside the container. In addition, the image analysis can provide the zipper teeth contoured pathway which can be used for the zipping action pathway. Again, x, y, z coordinates can be determined at pre-determined intervals along the zipper pathway. Such example implementation for an automated zipping can provide a more optimal pathway by which the system may have the maxim kinetic transfer of energy from multi-axis zipping device, through the end effector, and to the zipper slider body while minimizing the frictional forces created between the zipper slider body and teeth or elements.
      The container contents can be analyzed to determine if any corrective action is required prior to initiating the zipping process, including for example:
         Have container content 3050 object(s) been identified
         Are any of the content(s) 3050 obstructing opening 3030 of container 3000 such as to prevent closing 30400 of the container 3000, as illustrated in an example of FIGS. 20A and 20B.
         What is the position(s) of the container contents
         Can the container objects be successfully adjusted or removed to close the container
         Are there any identified object(s) which may impact the container apparatus motion
      Based upon such analysis a determination can be made as to whether content tendering is required, or the container closing/zipping process may continue.
Step 2, Closing preparations, is a process by which a container can be prepared for closing. The attributes analyzed by the VPU or other means can be evaluate and the necessary actions can be performed to move container to the closing or zipping stage.
   If container content tendering is required, the multi-axis device with attached end effector can adjust positioning of impacting container contents as determined by vision system or sensors. This may include, but not be limited, to picking and placing, pushing, pulling, bumping, and knocking the contents of the container creating any closing obstruction. In addition, container adjustments may be made by exerting force to the container itself whether by vibrating the container, gripping the container and manipulating the container surfaces to adjust contents, or stretching and relaxing the container via use of container grippers.
   Once the container content obstruction is removed the container can be clamped and tensioned at one or more points to provide stability for closing. The mechatronic clamps can be positioned vertically and laterally based upon coordinates provided by the VPU and the container can be clamped. According to example implementation, a container may not need to be clamped and/or tensioned, for example due to container rigidity, or any other factors such that clamping and/or tensioning may not be required to achieve closing. Additional inspection of the clamping (if needed), zipper slider body positioning, and zipper closing paths can be performed as needed for any correction required due to any one or more of clamping, tendering, or tensioning of the container.
Step 3: Closing process, is the process by which the container can be closed as can be determined by the VPU or other means.
   FIGS. 21A-21E illustrate an example implementation of disclosed embodiments where, using data provided by the VPU, the multi-axis zipping device moves the zipper body acquisition end effector 2000 into place by inserting the ends downward and rotating into place inside the container 3000, as illustrated in the example of FIGS. 21A-21C. The end effector 2000 then clamps onto the slider body 2040 of zipper 4020. As illustrated in the example of FIGS. 21D and 21E, the end effector 2000 is then guided along the three-dimensional pathway 4000 determined by the (VPU) until it reaches the end of zipping process to close the container 3000. In an exemplar implementation, end of a zipping process allowing room, for example 25 mm to 70 mm, to extract the end effector 2000 from the container 3000 at the end of zipping process. In an example implementation, should a measurable force which exceeds a configurable or dynamic threshold, measured in fractional newtons, be detected, the motion of the zipper body end effector will be altered accordingly to overcome any anomaly resistance. This may include, but not be limited to, acceleration, deceleration, velocity changes, incremental trajectory change(s), oscillation, rotation (yaw, pitch, roll), vertical, lateral, horizontal, or reversal. The end effector then releases the zipper slider body and is extracted from inside the container. Another image can be acquired and analyzed by the camera system and the (VPU) can determine whether the zipping action was successful. Upon success the (VPU) notifies the apparatus and the container is ready for the next stage. If the zipping action is not successfully the process is repeated.

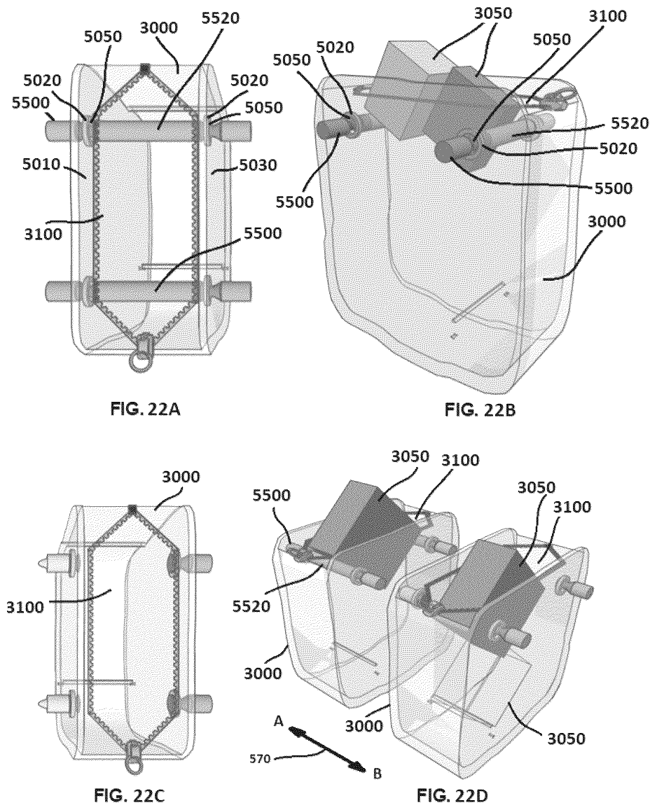

Referring to illustrative examples of FIGS. 22A-22D, an example embodiment is provided that can be used for containers, such as containers 3000 illustrated in FIGS. 20A-20B and 21A-21H. As illustrated in FIGS. 22A and 22B, such containers 3000 may incorporate two or more grommets 5020 defining openings 5050 in opposite sides 5010, 5030 of a container structure 3000 whereby such containers can be deployed in various systems to be supported by, and/or transported on, a tusk 5500 comprising a supporting rod 5520 inserted through the grommets 5020 on both side 5010, 5030 of container 3000. As illustrated in FIG. 22B, a configuration that includes a tusk 5500 comprising a support rod 5520 interferes with the flow of content 3050 into container 3000 at least due to supporting rod 5520 at least partially obstructing the opening 3100 of container 3000.

An example embodiments provide a configuration that can address the container content flow interference of the tusk. As illustrated in FIGS. 22C and 22D, content(s) 3050 which are caught by the tusks 5500 easily pass though the container opening 3100 of a “tuskless” design, which for example does away with a shaft 5520 at least during filling of container 3000 with content(s) 3050 via opening 3100. Such “tuskless” design (the term “tuskless” used herein for ease of understanding and not as a limitation) can improve the performance of the filling and closes processes.

Example implementation of disclosed embodiment provide a system and methodology comprising migration 5700 from a tusked container support configuration A to a tuskless container support configuration B, and vise-versa, as illustrated diagrammatically in FIG. 22D. A tuskless container support configuration can provide an opening design which can improve access via container opening area 3100 for filling container 3100 with articles 3050 without tusk interference. In a certain example implementation, a migration process utilizes various principles including but not limited to a method and apparatus where tusks can be configured to selectively retract and extend, or to include a structure that can be selectively extended or retracted. In a further example implantation, a system and/or methodology can be provided to prevent the container grommets 5020 from moving or slipping off the tusks 5050 during the migration process.

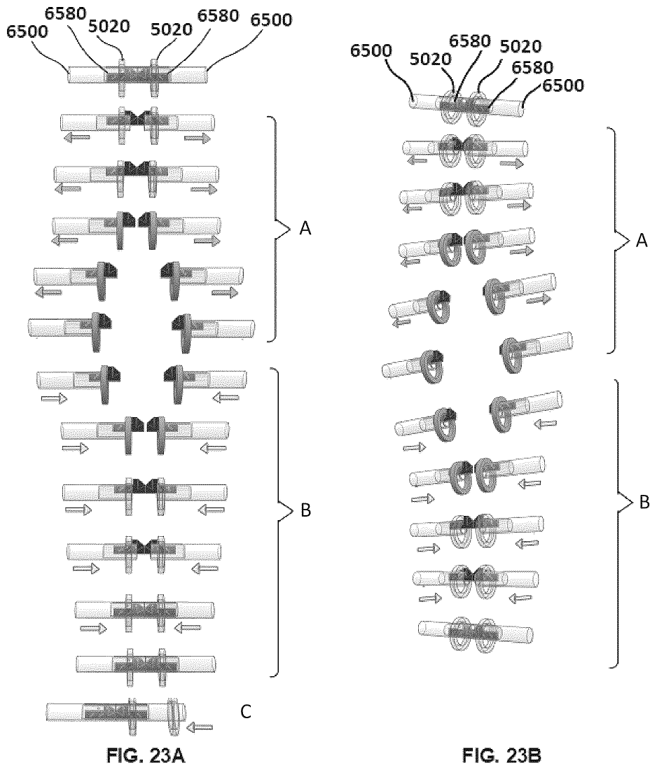

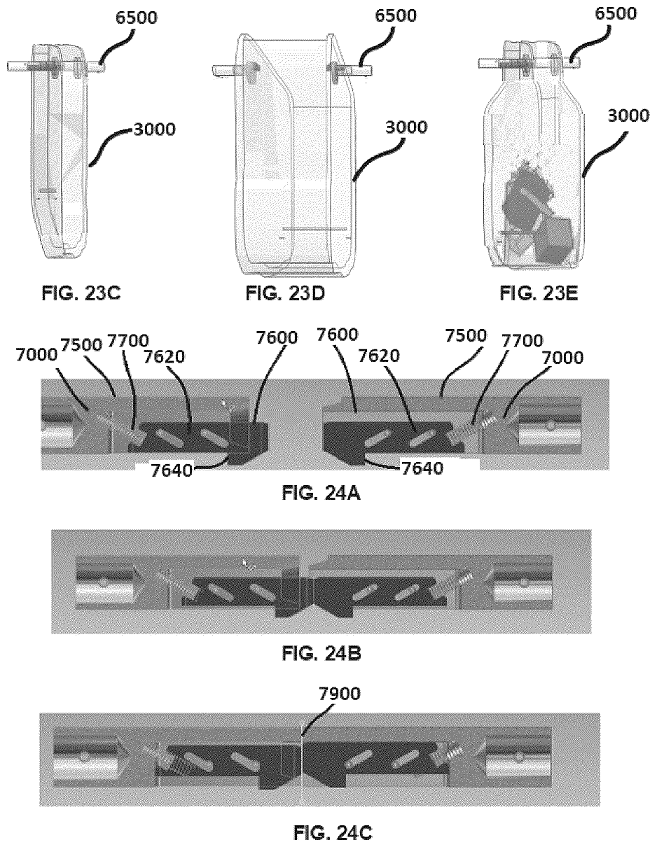

Referring to FIGS. 23A-23E, according to an example implementation one or more tusks 6500 extending through opposite sides 5010, 5030 of container 3000 is/are configured to separate and rejoin, for example by retracting (stage A) or extending (stage B) at least a portion 6580 of a tusk 6500 passing through a grommet 5020 (see FIGS. 23A and 23B, which is a perspective view of FIG. 23A). When the tusks 6500 are in a joined configuration, container 3000 can be supported by, and/or transported on, and/or removed from, tusk 6500 (stage C). FIG. 23C illustrates an example of an empty container 3000 supported by, and or transported on, one or more tusks 6500 in a joined configuration (for example prior to stage A). FIG. 23D illustrates an example of an empty container 3000 supported by one or more tusks 6500 in a separate configuration (for example after stage A and prior to stage B), where container 3000 is positioned to be filled with content 3050. FIG. 23E illustrates an example of a filled container 3000 supported by, and/or transported on, and/or removed from, one or more tusks 6500 in a joined configuration (for example in stage C).

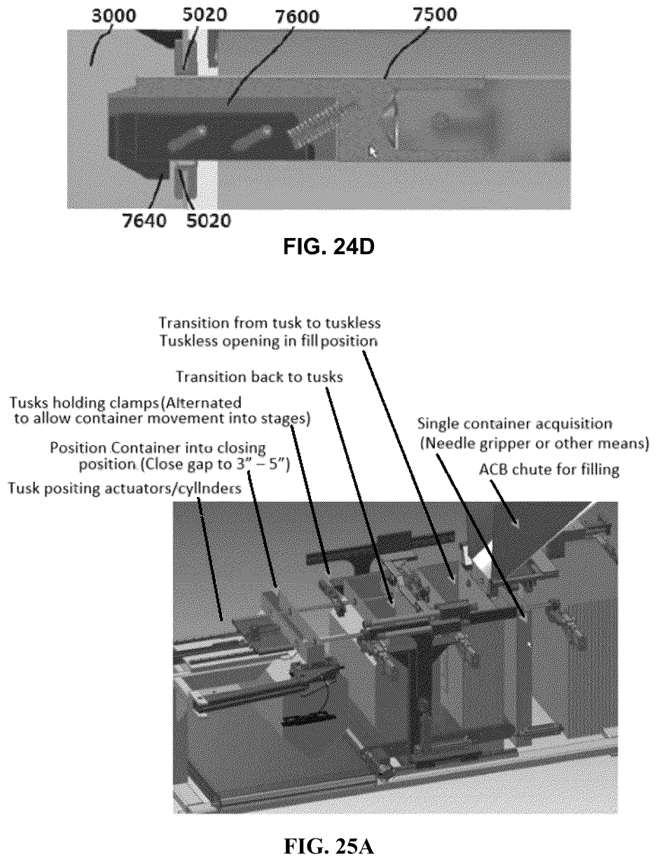

Referring to FIGS. 24A-24D, an example implementation, provides a system and/or methodology, for preventing container grommets 5020 from moving or slipping off the tusks 7500 during the migration process 5700, comprising a configuration, which can be referred to without limitation as a dog or a nubbin, to lock container grommets 5020 in place during the full opening of the container, such as show in the example of FIG. 23D, and transition from tusk to tuskless operation, such as shown in FIGS. 23A and 23B. In an example implementation, dogs or nubbins configuration comprises an actuated mechanism 7000, which can be positioned inside the tusks 7500, such that a dog or nubbin 7600 can be actuated by a compression spring or other biasing means 7700. In a further example implementation, dog or nubbin 7600 can comprise a base portion 7620 disposed inside a hollow tusk 7500 in communication with actuated mechanism 7000, and a retaining portion 7640 disposed outside tusk 7500 in communication with grommets 502.

As illustrated in example of FIG. 24C, while the tusks 7500 are in the closed, or contacting position, the dogs or nubbins 7600, including retaining portion 7640, can remain retracted inside the tusks 7500, which allows the container grommets 5020 to pass over the dogs or nubbins 7600, for example to be positioned such that the center line of the container can be substantially aligned where the tusks 7500 meet, or at a tusk contact point 7900. Once a container is in position, retaining portions 7640 of dogs or nubbins 7600 are pre-positioned on the inside of the partially open container such that the grommets 5020 are positioned on the outside of the retaining portions 7640 of retracted dogs or nubbins 7600 (see for example, top of FIGS. 23A and 23B prior to Stage A). As illustrated in the example of FIGS. 24A, 24B and 24D, when the tusks 7500 begin to separate, the dogs or nubbins 7600 are extracted such that retaining portions 7640 are outside of the tusks 7500 and engage grommets 5020 (see for example Stage A of FIGS. 23A and 23B).

Example implementations, as shown for example in FIGS. 24A-24D, can comprised a left and a right tusk 7500 component. In an open or separated tusk position of such a configuration, pins or retaining portions 7640 protrude outside the walls of at least partially hollow tusks 7500. When the left and right components are aligned and make contact, the pins 7640 are forced to retract back inside the tusks 7500, as illustrated in example of FIG. 24C (see also example Stage B of FIGS. 23A and 23B). The pins 7640 can be designed such that when they protrude outside the tusks 7500, container grommets 5020 are locked into place, as illustrated in example of FIG. 24D, while the container 3000 is fully opened by the process of separating the left and right tusks 750 (see also example Stage A of FIGS. 23A and 23B).

In still further example implementation, methodologies and configurations can be provided individually or in any combination where: the tusks may be precisely aligned to one another for rejoining; the tusks may be supported during separation and rejoining, for example to accommodate heavier containers; container content obstruction during or after filling of a container is detected, for example to mitigate adverse effect on a tusk rejoining operation; a successful tusk rejoining is confirmed by a visual, audible, and/or a tactile indication; and/or containers may be transitioned without opening the container and/or a migration operation from a tusked to tuskless configuration can be bypassed.

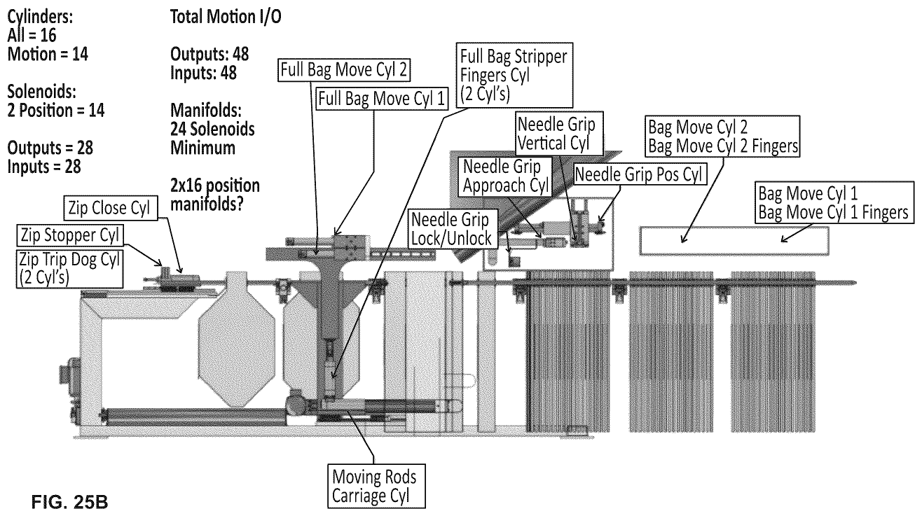

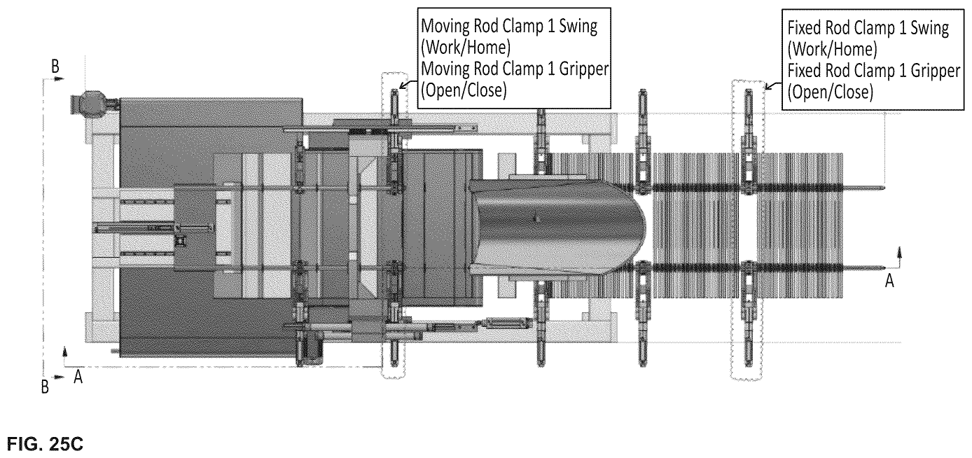

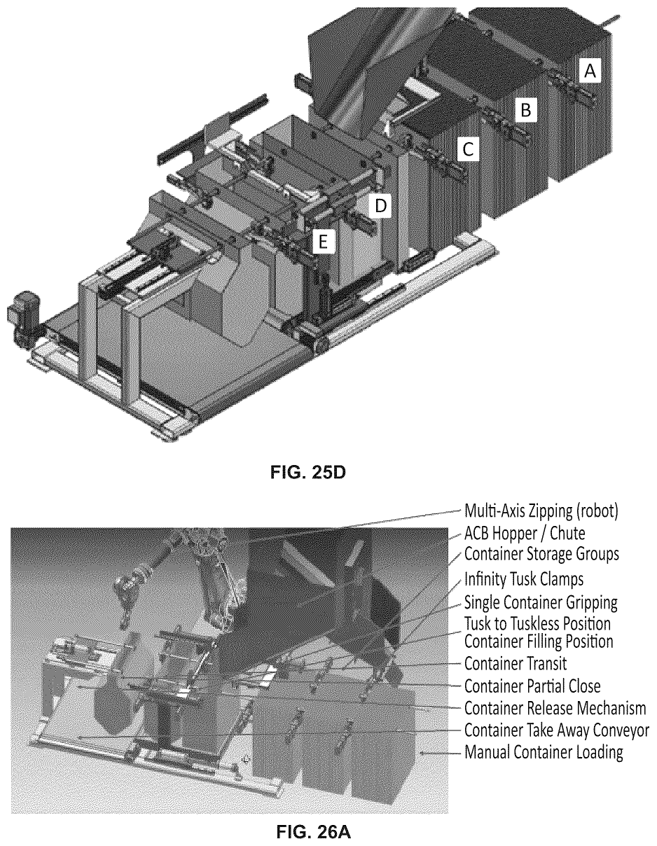

An illustrative example of a system implementing tusk to tuskless methodology, including example implementations of certain system components is provided in FIGS. 25A-25D, where 25A shows an example configuration with respect to a hopper, such as an ACB hopper of a system disclosed in U.S. Published Patent Application Pub. No. 20230159281, where FIG. 25B-25D show a side view, a top view, and an isometric view, respectively, of an example thereof.

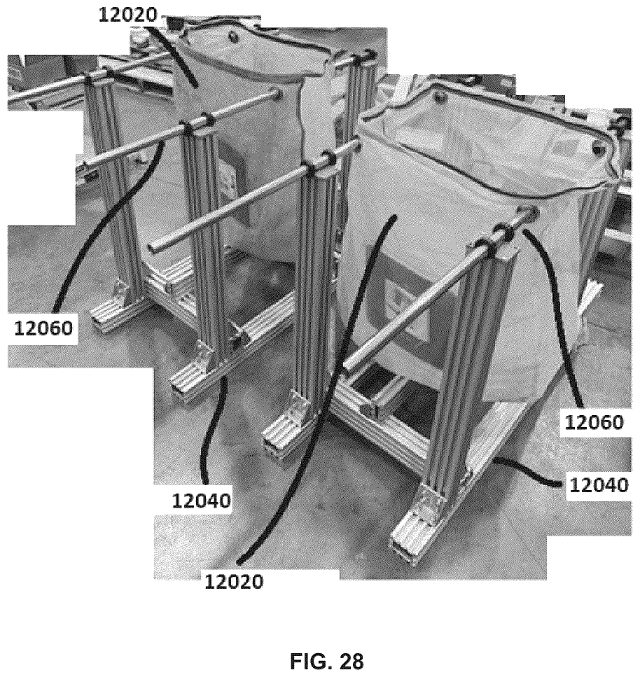

A system according to yet another example embodiment can be configured with respect to a package source other than an ACB hopper, such as for example a smart bin disclosed in U.S. Published Patent Application Pub. No. 20230159281, or directly with respect to an exit location of a sorter, and include for example:
a container storage location, where for example in a case of flexible containers such a location may include system component for supporting flexible containers, for example in container storage group or groups stored in a flat configuration to facilitate utilization of the storage space, a tusk configuration for supporting and transporting individual containers from the container storage group(s) to a container staging location, including for example a bag isolating gripper, as shown in FIG. 26D to facilitate acquisition of a single flexible container, such as a bag, from storage groups;
a container staging location, where according to an example implementation of the example embodiment in a case of flexible containers, such a location may include system components or a mechanism for migrating from a tusk to tuskless configuration to facilitate opening of the container, as shown in an example of FIG. 28 where individual flexible bags 12020 are mounted on individual stands 12040 that can provide a tuskless configuration and/or a mechanism that can transition between tusk and tuskless configuration 12060;
a transport mechanism for
   transferring of an open container from the container staging location to a designated position with respect to one or more of a plurality of smart bins, or directly with respect to sorter exit or exits, such that the content of the designated smart bin(s), or directly from the sorter exit, can be deposited into the open container, and
   then transferring the open container filled with the deposited content to a container closing location,
   where according to an example implementation of the example embodiment such a transport mechanism may comprise one or more autonomous vehicles (AVs), where each of such AVs can be configured to transport one or more open containers from one or more of the container staging locations to one or more designated positions with respect to one or more of the smart bins, or directly with respect to the sorter;
a container closing location, where according to an example implementation of the example embodiment in a case of flexible containers, such a location may include system component or a mechanism for staging the container for closing including a mechanism to facilitate transition from a tuskless to a tusk configuration for supporting the flexible container, a partial close mechanism, and a multi-axis tool, such as a zipping tool, for closing the container, for example as shown as described above, for example with reference to FIGS. 27A-27C.

An example embodiment of a methodology, employing a system configured with respect to a smart bin disclosed in U.S. Published Patent Application Pub. No. 20230159281, or directly with respect to a sorter, as described in above example embodiment, can include the following combination of steps:
Providing a bag stand (such as in FIG. 28) in a tuskless configuration at each exit off of a sorter, where each such bag stand in a tuskless configuration would arrive at the exit location with a bag already automatically installed and open (for example at a container staging location).
Once a bag is deemed FULL, designating that bag for pickup, for example by indicating that lane of the sorter if turned OFF so that it would not receive additional packages. Pickup would occur when an AV acquires (for example, attaches to or picks up) the bag stand comprising the FULL bag in order to remove it from the sorter.
Providing a group of AVs, for example in a queue, each AV configured (for example, by attachment, or as a support) with a bag stand comprising an empty bag, waiting to move into any position that would become ‘open’ due to a ‘full’ bag. Once an empty bag is in position under a sorter exit location, that sorter exit location can be turned ON, allowing packages to once again exit the sorter at that specific location.
Transferring FULL bags to a bag closing location, such as a bag zipping location, where FULL bags would be zipped closed with a robotic zipping technology as described infra.
Disengaging the FULL closed, for example zipped, bag from the AV, and/or from the bag stand, for further transport of the FULL closed bag, for example on a collector belt.
Directing AV to a different location, for example a container staging location, where a the AV can be assigned, or attached, to an open empty bag, for example AV can be furnished (by attachments, or as a support) with a bag stand such as the bag stand shown in an example of FIG. 28 described above.
Placing the AV with the empty bag (for example, with a bag stand comprising an empty bag), a so called REPLENISHED AV, in a queue, or in a group of AVs waiting to move under the sorter when a FULL bag is removed.

According to example implementations of the disclosed embodiments, more than one queue for REPLENISHED waiting AVs and/or more than one container closing, for example bag zipping, station can be provided, for example to facilitate meeting certain rate goals.

According to example implementation of disclosed embodiments, data can be collected dynamically for artificial learning with reinforced and unsupervised learning during any and all stages of operation. The VPU image analysis can provide the raw data both pre and post zipping. This data can include the predisposition of the container, contents, zipper components, zipper pathway models, and subsequent results or success rates for each container. In an artificial intelligence module (AIM), using one or more algorithms, including but not limited to, logistic regression, a statistical success to classify and create predictive models for future motion iterance's can be evaluated. A machine learning module (MLM), can be implemented to compare and apply these classifications to create new motion control decision trees for continuous performance improvements. A separate independent simulation model based upon the AIM and MLM calculations can be used for reinforcement.

Non-limiting examples of potential fields of uses of example embodiments of the disclosure include:
Garment industry—Labeling/Folding clothes, and containerizing
Fulfilment center—Packaging/Closing/Labeling containers. Just about anything
Food/Beverage Packaging—Filling, closing, labeling bags of anything from pet food, to powdered laundry detergent, to candies/snacks, frozen vegetables, fruits, sugar, flour, rice, etc.
Agricultural—Grain, Feeds, Fertilizer, soil, pesticides
Building/supply—Concrete, Sand, landscaping material, hardware-nuts-bolts-nails etc.
Baler—Pine straw, straw, wheat, other
Containerizing Recyclables
Container types
Cartons, crates, totes, baskets,
mesh containers
Plastic bags
cloth bags
Closing mechanisms
Stitching
Heat sealing
Velcro
Zipper
Clamped
Magnetic
Compression
Folded
Zip locked
Draw string
Banded
Zip Tie

An example of a methodology according to example embodiments of the disclosure can include the following outline of processes:
a) Determine type of container by evaluating the (size/shape/volume/color)
   a. Used in determining the length of stroke of the apparatus cylinders for container positioning and container opening dimension
b) Detect container present
c) Analyze container status upon filling
   a. In or out of proper filling location
   b. Closure mechanism and or closure attachment status (Present, missing, damaged, unknown)
   c. Container overfilled with contents
   d. Closing mechanism obstruction (Container itself and contents)
d) Analyze closing mechanism and or attachment
   a. Presence, condition
   b. Placement (horizontal, vertical)
   c. x, y, z plane (pitch, yaw, roll)
e) Analyze trajectory for closing
   a. Profile/contour of closing motion pathway (x, y, z)
f) Provide point data for multi-axis closing/zipping device
g) Analyze closure
   a. Closed, Partially Closed, Open
h) Other
   a. Provide statistical data and real time system feedback for engineering and operations teams
   b. Utilize artificial intelligence and machine learning algorithms to improve accuracy and performance

### (Multi-Axis Device Closing/Zipping)

a) Determine motion path to position specialized end effector to acquire zipper slider body or attachment
b) Initiate zipper slider body or slider body attachment acquisition by specialized end effector
c) Determine multi-axis motion path to follow optimal flexible container contoured closure pathway as determined by vision system
d) Initiate and complete closing or zipping motion
e) Determine specialized end effector extraction motion pathway
f) Extract specialized end effector out of and away from flexible container
g) Position specialized end effector back to home position
h) Other
   a. Provide statistical data and real time system feedback for engineering and operations teams
   b. Utilize artificial intelligence and machine learning algorithms to improve accuracy and performance

### (Multi-Axis Device Tendering)

a) Adjust positioning of impacting container contents as determined by vision system or sensors
   a. Including but not limited to picking and placing, pushing, pulling, bumping, and knocking
b) Adjust container by applying lateral forces to flexible material
c) Grip flexible container and manipulating material to adjust contents
d) Stretch or relax container via use of container grippers

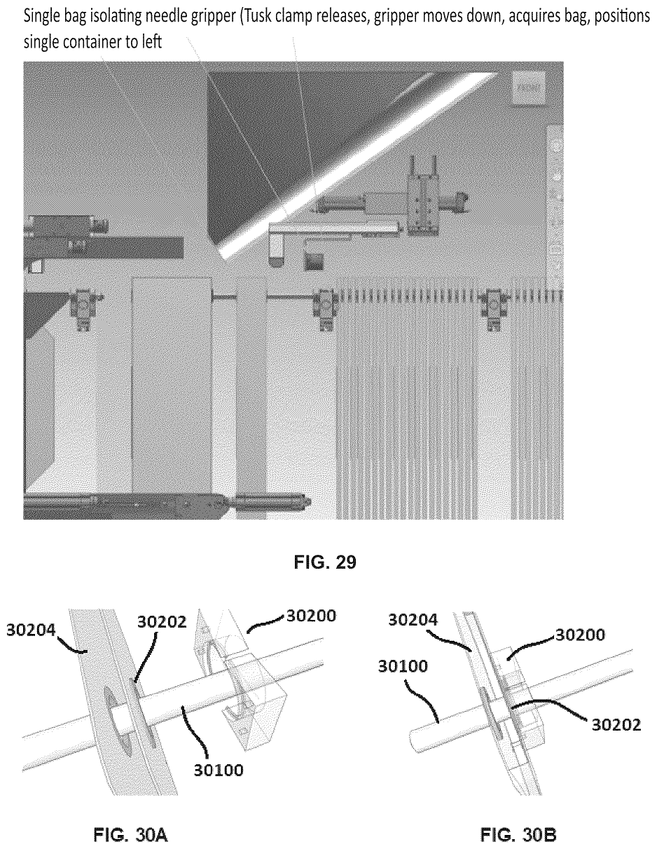

FIG. 29 illustrates a system that can be configured with respect to a package source, including for example an automated consolidated bagging (ACB) hopper or chute described infra, and includes a tusk configuration, such as for example an infinity tusk, for supporting and transporting individual containers from storage groups to a container filling position, where a bag isolating gripper, for example a needle gripper, can be provided to facilitate acquisition of a single flexible container, such as a bag, from storage groups.

Referring to FIGS. 30A and 30B, a system according to an example embodiment of the disclosure can be configured to isolate one individual container from the groups of stored containers on infinity tusks 30100 by a mechanism 30200 that can utilize one or more grommets 30202 attached to the containers 30204 to isolate and move the container to the next processing stage in systems such as for example and without limitation, automated sortation systems described above, in U.S. Published Patent Application Pub. No. 20230159281, and/or in U.S. Pat. No. 11,743,169. According to an example implementation, a mechanism (which can be referred to for convenience, and without limitation, as a “Grommet Gripper” or “grommet gripper”) can be implemented in a configuration that takes into consideration that a relative positioning of a container's grommet on an infinity tusk can be more consistent than that of other elements of a container. Based on the foregoing, a gripping device that utilizes a container's grommet can facilitate the more accurate positioning of the gripping device to acquire a single container. An example implementation of the disclosed embodiments provides a gripping device with a gripper designed to clamp over the container grommet while not damaging either the grommet or container, as diagrammatically illustrated in a non-limiting example of FIG. 30B.

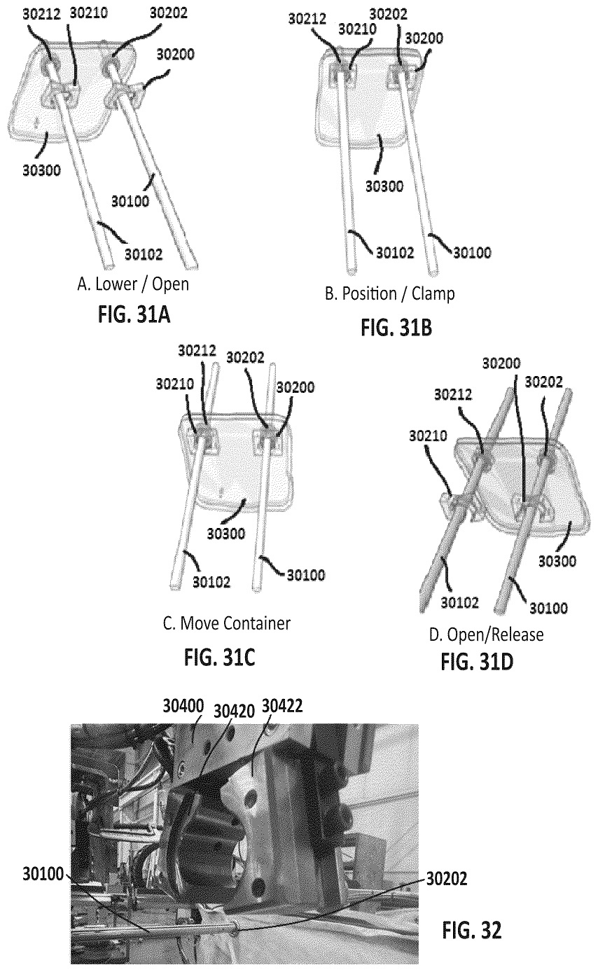

Referring to FIGS. 31A-31D, according to an example implementation, a Grommet Gripper 30200 starts in an open position and is automatically lowered into the vertical, or Z axis position, as illustrated in a non-limiting example of FIG. 31A. As illustrated in examples of FIGS. 31A-31D, two grommet grippers 30200 and 30210, for example operating synchronously and/or independently, and configured with respect to respective tusks 30100 and 30102, can be utilized to grip grommets 30202 and 30212, respectively, of container 30300, and move container 30300. A control system according to example embodiments, can then utilize various methodologies to move a Grommet Gripper 30200 into a position with respect to a grommet 30202 to facilitate gripping of the grommet 30202 (and, for example, to move grommet gripper 30210 into position with respect to grommet 30212 to facilitate gripping of the grommet 30212).

For example and without limitation, an apparatus controls system can comprise hardware and/or software having stored thereon a servo positioning of the Grommet Gripper 30200 from a previous bag 30300 acquisition. In an example implementation, an average grommet and container width can be subtracted to calculate a near position of a container to be acquired. The apparatus controls system can then facilitate movement of Grommet Gripper 30200 and/or 30210 to within a predetermined distance from the calculated near position where one or more position sensors, such as for example a distance sensor, can be utilized to more precisely guide grommet gripper 30200 and/or 30210 into the final position for gripping the grommet 30202 and/or 30212, respectively, as illustrated in a non-limiting example of FIG. 31B. In a further example implementation, if a previous container position is not known, for example due to a system reset or an absence of containers available, the system can rely, for example solely, on a distance sensor for positioning the grommet gripper 30200 with respect to a grommet 30202 and/or grommet gripper 30210 with respect to a grommet 30212.

Once in position, the controls system can close the grommet gripper 30200 and/or 30210, such that for example grommet gripper 30200 and/or 30210 clamps around the grommet 30202 and/or 30212, respectively, of container 30300, as further illustrated in a non-limiting example of FIG. 31B. This can facilitate a secure robust method of separating a container 30300 from the other containers, such as for example groups of containers stored on infinity tusks, and/or moving individual containers, as illustrated in a non-limiting example of FIG. 31C.

In yet further example implementation, grommet gripper clamping process can be verified by one or more sensors to ensure secure clamping of grommet gripper to respective grommet. For example, an apparatus controls system can be configured to move the container into the next stage for processing after successful clamping has been validated. In still further example implementation, an apparatus controls system can be configured to confirm successful container separation, for example utilizing a camera vision/vision processing unit (VPU) system such as a system described above and in U.S. Published Patent Application Pub. No. 20230159281. The grommet gripper 30200/30210 then opens, releasing the grommets 30202/30212, and for example grommet gripper 30200/30210 can be raised so as not to obstruct the path of the container or other ongoing parallel processes, as illustrated in a non-limiting example of FIG. 31D. This process, as illustrated in, and described with reference to, FIGS. 31A-31D can then be repeated for all subsequent containers.

Referring to FIG. 32, according to an example implementation, a grommet gripper 30400 (illustrated in the example of FIG. 32 in an enlarged perspective view), such as grommet gripper 30200 and/or 30210, can comprise two mechanical components 30420 and 30422 sized for gripping of, or engagement with, container grommets 30202/30212. These two components can be articulated, for example individually or together, to open and close as commanded by a controls system. In a further example implementation, one or more grommet grippers 30400 can be attached to one or more control arms which may be lifted and lowered for vertical positioning with respect to one or more grommets 30202 and/or tusks 30100. In yet further example implementation, one or more grommet gripper arms can attach to a linear motion system to move the respective grommet grippers bilaterally either in the direction of the container to clamp onto the grommets or the opposite direction to isolate the container by pulling the container's grommets along the infinity tusks. In still further example implementation, sensors can be attached to the grommet gripper and/or the arm to verify commanded opening and closing of a respective one or more grommet grippers. A camera vision system/VPU, such as a system described above and/or in U.S. Published Patent Application Pub. No. 20230159281, can also be implement to assist in a determination if a successful container separation has been achieved.

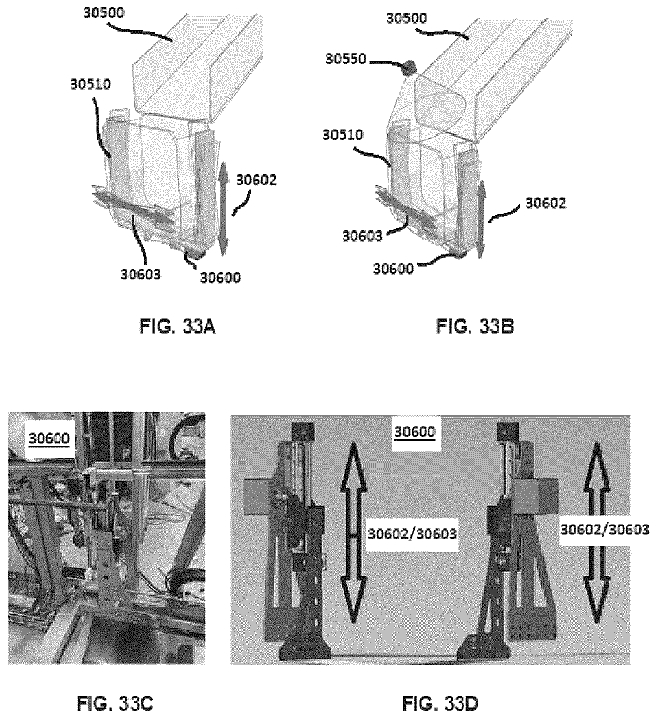

Referring to FIGS. 33A, 33B, 33C, and 33D, according to an example embodiment, automated sortation systems, such as those described above, in U.S. Published Patent Application Pub. No. 20230159281, and/or in U.S. Pat. No. 11,743,169, can be configured to utilizes a modified automated consolidated bagging (ACB) chute 30500 to fill the containers 30510 with groupings of packages. As packages/content fill the containers 30510 this may occasionally result in content overfilling the container. To maintain performance, systems according to example implementations of the disclosed embodiments can comprise an automated mechanism to facilitate package tendering.

An example implementation can be configured to utilizes, for example omnidirectional, motion 30602/30603 of the container 30510 as it is filled to facilitate improved settling, placement, and organization of the container contents, for example to limit overfilling and subsequent human interventions which may be required. Referring further to a non-limiting example of FIGS. 33A-33D (see also FIG. 25A), a mechanical device 30600 can be provided to physically manipulate one or more facets of the container 30510 in such a manner as to cause movement of the content within the container, for example to allow gravity to resettle the contents. This process can be repeated with varying motion profiles, for example until the entire contents of the container settle such that, for example, the container may be fully closed.

According to a further example implementation, a package tendering process can be automatically started during an entire filling process or as required. In yet further example implementation, an industrial camera system and a vision processing unit (VPU) 30550, such as a system/VPU described above and/or in U.S. Published Patent Application Pub. No. 20230159281, can be used to determine if the contents of the container have overfilled the container and could obstruct the ability to automatically close the container. This task can be performed and/or completed both during and after the filling process. According to still further example implementation, an automated tendering process can be continued until the VPU 30550 notifies an apparatus controller, such as a system controller descried above and/or in U.S. Published Patent Application Pub. No. 20230159281, of success. In yet another example implementation, in the event the package tendering is unsuccessful during filling, the system can be configured to automatically change motions profiles 30602/30603 of mechanical device 30600, for example based upon feedback from the VPU 30550. Potentiation changes to motion profile can include, but are not limited to, any of: range of motion, direction of motion, sequencing of motion, frequency of motion and motion velocity. In yet further example embodiments, empirical measurement parameters can be associated with a successful motion profile, which may then be logged and utilized for additional, for example artificial intelligence, training to develop more success profiles based upon feedback from the VPU.

In automated sortation systems described above, in U.S. Published Patent Application Pub. No. 20230159281, and/or in U.S. Pat. No. 11,743,169, there is a time period for packages or container contents to travel from, for example, the ACB cleated conveyor or other systems to the opening of the container on system. For example, such a time period can range from four to ten seconds in the current ACB system, and can limit the overall process time of an automated bagging system. To potentially reduce this content conveyance and drop time, example embodiments of the disclosed systems can comprise an automated stage gate chute.

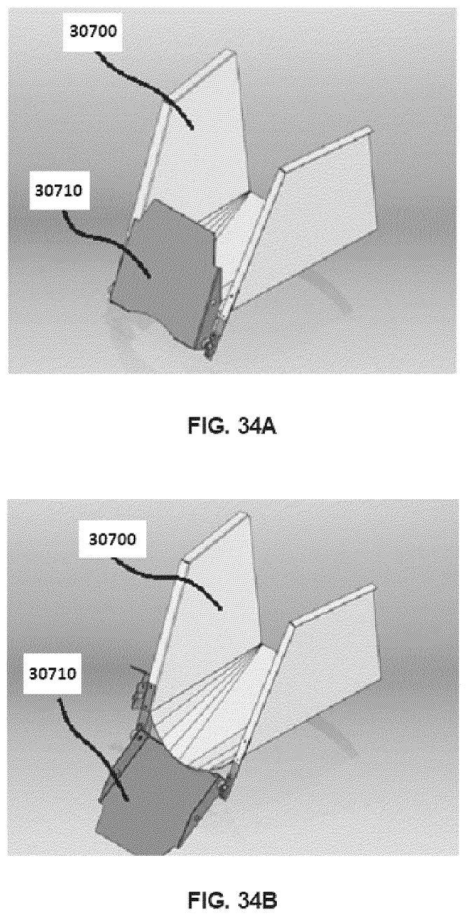

Referring to FIGS. 34A and 34B, according to an example implantation, a chute 30700, such as an ACB chute, can comprise an automated stage gate 30710 that can remain in a closed position, as illustrated in a non-limiting example of FIG. 34A, while container contents are released from the feed system. In parallel, a system controller, such as an apparatus PLC controller described above, in U.S. Published Patent Application Pub. No. 20230159281, and/or in U.S. Pat. No. 11,743,169, can transition an empty container into the filling position with respect to stage gate 30710, and open the container to receive the contents of chute 30700. In a further example implementation, while an empty container is being prepared to be filled, a feed system can release the container contents to fill the chute to be staged. The stage gate 30710 can prevent the contents dropping from the chute 30700 prematurely. Once the container to be filled is in place and ready for filling, the system controller can open the gate 30710, as illustrated in a non-limiting example of FIG. 34B, to release the contents into the empty container. In yet further example implementation, after the filling is verified by the camera/VPU system, for example system 30550, the gate 30710 will be transitioned back to a closed position and await the next cycle. In this manner, the container content conveyance can be reduced or eliminated. In addition, the container content drop time can also be reduced.

According to example implementations of the disclosed embodiment, chute system 30700 can comprise a custom fabricated gate 30710 which can seal the chute and prohibit all container contents from dropping. For example, mechanical hinges can be configured to allow gate 30710 to be automatically lifted and lowered for content control. This gate design could include but is not limited to vertical, lateral, hinge, slide, single, or multiple gate actuation. In still further example implementation, electrical or pneumatic actuators, for example controlled by a system controller, such as an apparatus PLC controller described above, can be configured to coordinate the content feeding and filling process. In still further example implementation, sensors can be configured to confirm whether the stage gate 30710 is in the appropriate, opened or closed, commanded position. In still further example implementation, a camera vision/VPU system, for example as described above, can be configured to determine if any container contents are hung up or caught in the gate 30710 or stuck on the chute 30700.

FIG. 25A illustrates an example of a system that can be configured with respect to a package source, including for example an automated consolidated bagging (ACB) hopper or chute disclosed for example in U.S. Published Patent Application Pub. No. 20230159281, and includes a tusk configuration where tusk holding clamps are configured to alternate to allow container movement into various stages. In example implementations, clamp arms may be required to support the infinity tusks 30100/30102 in such a manner that they may engage and disengage the infinity tusks. As the containers are supported by the infinity tusks by penetrating the container grommets, any mechanism supporting the tusks can prevent the containers from moving past said supports.

In order not to unnecessarily prevent the movement of the containers along infinity tusks, an example implementation can include a configuration where the infinity tusks' clamp arms are systematically engaged and disengaged to allow the container grommets to pass by one arm while continuing to support the infinity tusks. For example, by alternating which clamp arms are engaged and disengaged the containers may freely move along the infinity tusks.

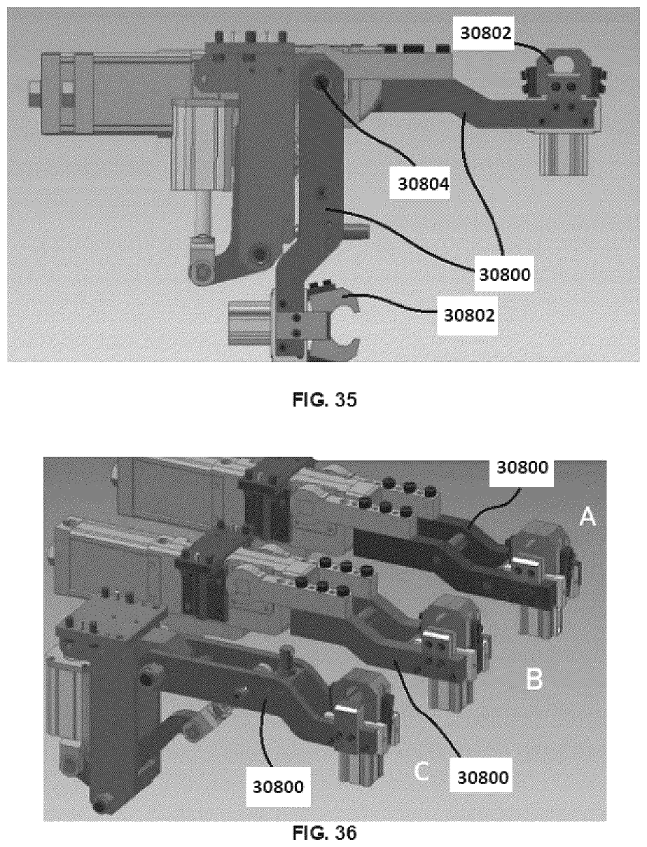

Referring to FIG. 35, according to an example implementation of the disclosed embodiments, clamp arms 30800 are either positioned vertically, “bypass” position, or horizontally, “engaged” position. In a further example implementation, each actuated arm 30800 also incorporates a clamp 30802 to hold infinity tusks in place. For example, each clamp position on the infinity tusk rods can be cut out to prevent the rods from moving perpendicular to the clamp arms 30800. In yet further example implementations, these cutouts can align with a block located directly under the clamps.

According to example implementations, as the containers move along the infinity tusks, and they encounter an engaged clamp arm 30800, the arm can be commanded by as system controller and/or PLC, such as those described above, to disengage and swing down into the “bypass” position. In yet further example implementations, a system controller and/or PLC can be configured to coordinate the clamp arm 30800 positions such that for every clamp arm in the bypass position both adjacent clamp arms are in the “engaged” position. According to further example implementations, clamp arms 30800 can work in pairs, where each arm is paired with its opposing clamp on the adjacent infinity tusk.

Referring to FIGS. 25D and 36, in an example implementation, if a clamp arm C is in the bypass position, then clamp arms B and D must be in the engaged position to support the infinity tusks and containers. Once clamp arm C swings down into the bypass position, the container is free to move past this position. Since the containers are supported by two parallel tusks, a system controller and/or PLC should be configured to coordinate the clamp arm pairs. As the container continues to move along its path, subsequent clamp arms are bypassed and engaged as described.

According to example implementations of disclosed embodiments, clamp arms 30800 can be comprised of, but not limited to, a control arm, a position actuator, joints 30804 to allow the vertical “bypass” and horizontal “engaged” positions, actuated infinity tusk or rod clamps 30802, sensors to confirm positions, and rod guide blocks which align with the infinity tusk cutouts. In an example implementation, a position actuator can be configured to move to allow the arm to swing up to horizontal and down to vertical positions. In further example implementation, the joints can be configured to allow the actuator, whether horizontal, rotary, or vertical, to move into both positions. In still further example implementation, the actuated clamps can be configured to grip the infinity tusks or rods for support, the sensors confirm whether the tusk or rod clamp is fully engaged for robustness and safety, and the rod guide block engages the tusk or rod cutouts to prevent the tusks from parallel movement to the clamp arms.

Referring to FIG. 36, according to an example implementation, infinity tusk clamp arms 30800 (A, B, C) support the tusks while the containers are moved from station to station in a system described above, in U.S. Published Patent Application Pub. No. 20230159281, and/or in U.S. Pat. No. 11,743,169. According to yet another example implementations, to facilitate a reduction in the overall width of a system, the clamp arm 800 may be constructed such that the control mechanism actuates with a vertical motion versus laterally or rotary, as further illustrated in a non-limiting example of FIG. 35.

According to example embodiments, configuration of a clamp arm 30800 can comprise a vertical actuated pneumatic or electrical actuator and, for example three, hinges to enable the swing motion required to engage and support the tusks. Such a configuration can advantageously prevent a mechanical control actuator from extending beyond the infinity tusks thus reducing the overall width.

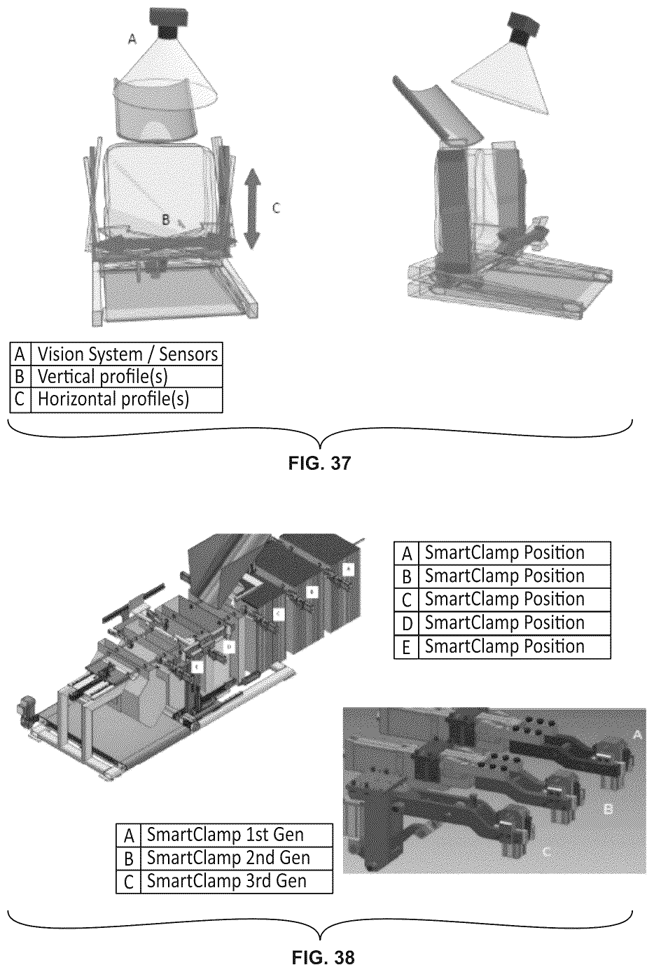

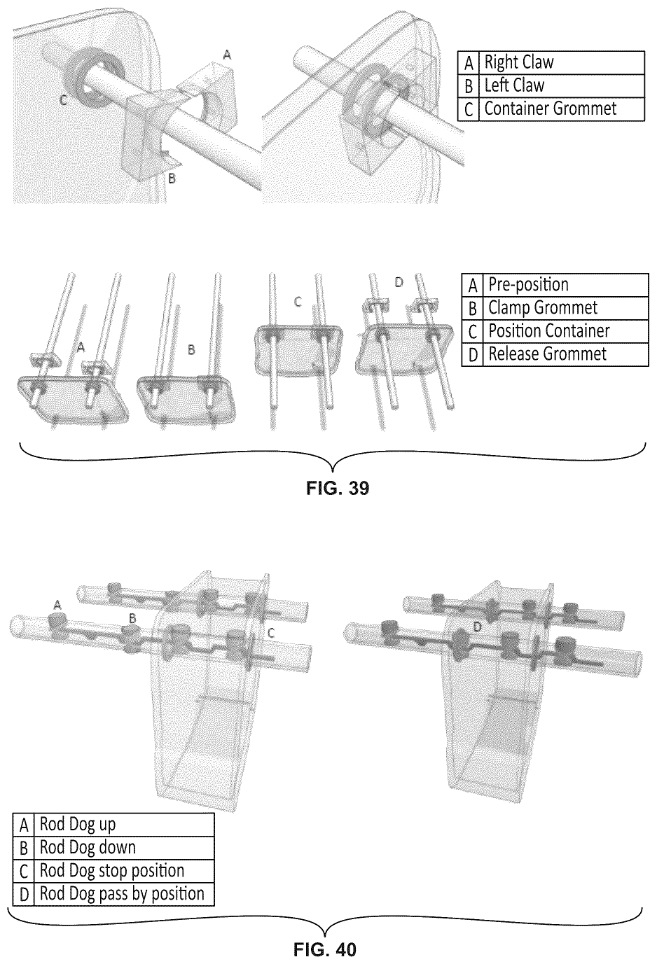

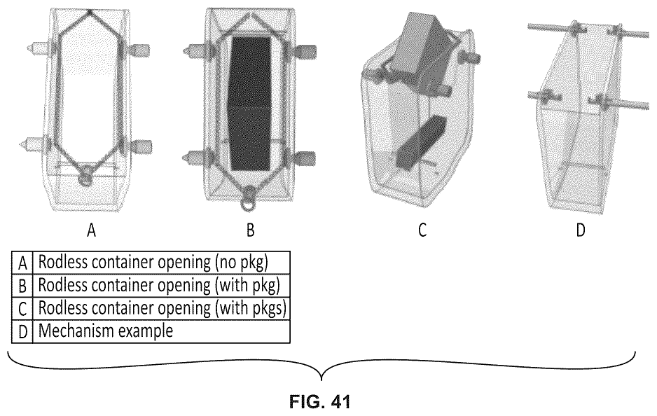

Further example implementations of disclosed embodiments, where descriptive terminology such as “SmartGrip,” “Smart Profiling,” “Smart Tendering,” “SmartClamp(s),” “SmartClaw(s),” “RodDog(s),” “SmartRod(s),” “SmartCart,” “SmartRack,” “StageGate,” and “SmartBagger” is provided for ease of understanding and reference and not as a limitation, include:
a. SmartGrip
   i. Container closing mechanism acquisition device(s) example, as illustrated in a non-limiting example of FIGS. 19A-19E.
b. Smart Profiling
   i. Software, equipment, and devices required to enable three-dimensional contour motion profile for container closing. (X, Y, Z, pitch, yaw, roll), as illustrated in a non-limiting example of FIG. 21D-21E.
c. Smart Tendering
   i. Software, equipment, and devices required to enable automated package tendering profile(s) actuation and methods of control, as illustrated in a non-limiting example of FIG. 37.
d. SmartClamp(s)
   i. Clamps for sequential alternating suspension of container rods, as illustrated in a non-limiting example of FIG. 38.
e. SmartClaw(s)
   i. Grommet acquisition and sequencer example, as illustrated in a non-limiting example of FIG. 39.
f. RodDog(s)
   i. Automated container positioners and stops example, as illustrated in a non-limiting example of FIG. 40.
g. SmartRod(s)
   i. Mechanism(s) and controls to open containers to eliminate rod interference with contents during the fill process, as illustrated in a non-limiting example of FIG. 41.

Further example embodiments of the present disclosure provide process and machine to enable a mobile robot to transport a cart or rack of containers, position them in front of the SmartBagger apparatus, move the cart or rack of containers in place, align the cart or rack tusks with the SmartBagger infinity tusks, join the cart or rack tusks and SmartBagger tusks together, latch and secure the cart or rack tusk union, automatically offload the containers from the cart or rack tusks onto the automated SmartBagger tusks, unlatch and separate the cart or rack tusks and SmartBagger tusks, eject the empty cart or rack, transport, and stage the cart or rack to be replenished containers.

Referring to FIG. 42, containerization for materials, products, packages, and other items requires containers. These containers can create numerous logistic issues and additional costs for industries. Containers must be acquired, stored, transported to the containerization process, and transported again to its intermediate or destination. This process can be labor intensive and costly to the industry. In addition, container logistic bottle necks may be created negatively affecting the containerization process itself.

An ACB, automated consolidated bagging system may require over a thousand containers per hour for each installed system. In an example of manual processes, containers housing product are emptied in one location (1), then the containers themselves are containerized by placing them inside one another or stacking them in small groups or piles (2). The containerized containers are then placed into yet another container for transportation such as a cart, gaylord or gurney (3). The containerized containers of containers are then manually transported to the ACB, automated consolidated bagging parcel containerization area (4). Next the containers are de-containerized (5) and staged for use on container processing racks, or bag stands, each holding approximately twenty-five containers (6). These racks are first staged nearby (7) and later manually transported to the appropriate SmartBagger position for use as required (8). Finally, the container processing racks (9) and the now empty containers for the containers, carts, gaylords or gurneys (10) need to be manually repositioned back to the starting point where the process is repeated throughout the operation.

This process is the same for the manual Automated Consolidated Bagging, ACB, except the racks are again staged at the manual operation station for use by the human operators. These ten process steps are repeated hundreds or thousands of times per day in operating facilities.

A method to improve on, or essentially eliminate, this repetitive, laborious, costly, and ergonomically challenged processes is required. Mobile devices or robots, whether autonomous or guided, are well suited to assist in this task. Mobile robots can significantly reduce the manual processes of transportation. In addition, the use of these robots can also eliminate other elements in the containerization logistic process altogether. In conjunction with the mobile devices or robots a series of mechanical devices to automate this process are required for integration with the Automated Consolidated Smalls system and/or SmartBagger apparatus.

Further example embodiments of the present disclosure may address above-noted drawback and/or disadvantages. Example implementations of such further example embodiments of the present disclosure provide a system whereby container rack(s), either mounted to a mobile robot or in tow, can automatically dock with the SmartBagger system, which can reduce time and eliminate multiple process steps. For example, referring to FIG. 42, manual steps two, three, four, five, seven, eight, and nine can all be fully automated within the scope of the present disclosure. Example implementations of disclosed embodiments can be configured for robotically transporting, staging, and automatically docking mobile container racks with the SmartBagger system, or essentially any system where one or more containers need to be transported and/or staged.

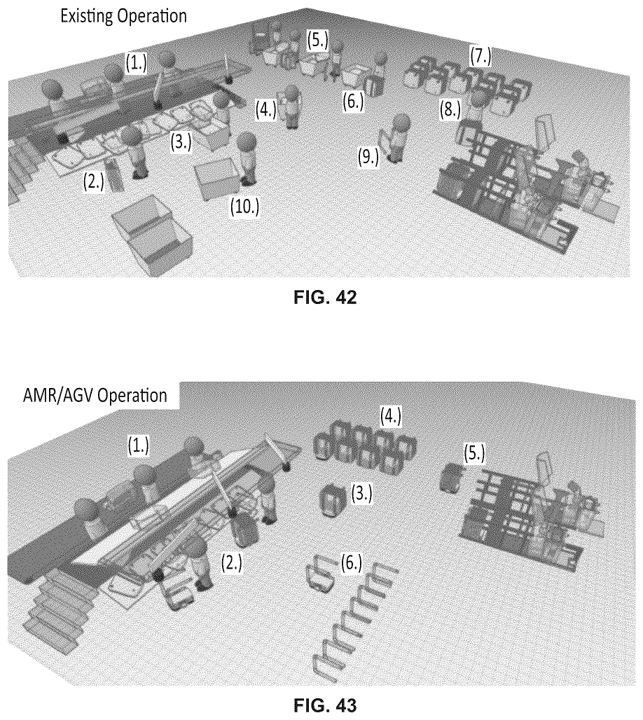

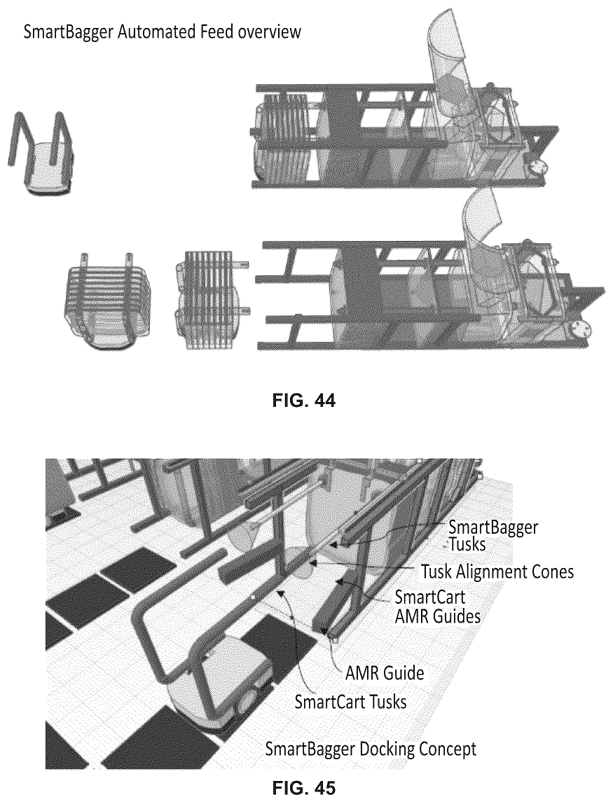

Referring to FIGS. 43-45, according to an example implementation of example embodiments of the present disclosure, an AMR/AGV SmartBagger Loading Operation may, for example, reduce operational staffing, in a configuration where, for example:
1. Container Emptying process remains essentially unchanged.
2. The employee(s) will no longer containerize containers for transport but rather load containers directly onto a SmartCarts to be positions by the AMR or AGV system.
3. AMR/AGV transport of the SmartCart to either a SmartBagger (5.) or to a full SmartCart staging area until requested by the SmartBagger system.
4. SmartCart Staging Area.
5. SmartBagger Autonomous Loading.
6. AMR/AGV transport of an empty SmartCart to either an empty SmartCart Staging area or to an employee to reload the SmartCart.

Example implementations can provide a system that may incorporate, but is not limited to, autonomous or guided mobile robots or vehicles, AMR(s) or automated guided vehicles, AGV(s), a vision system or sensors to monitor the mobile robot positioning and docking, omnidirectional cart(s)/rack(s) to hold staged containers, tusk positioning and latching, container offloading onto the SmartBagger, unlatching the joined tusks and undocking the robot, a mechanism to assist in guiding the robot into its final position, a mechanism to draw the robot into its docking position, a mechanism to guide the cart or rack tusks to align with the SmartBagger tusks such that they may be joined, a mechanism to lock the robot into position while the containers are offloaded from the cart or rack, sensors to detect successful operation(s), a controls/software system which integrates with the SmartBagger system.

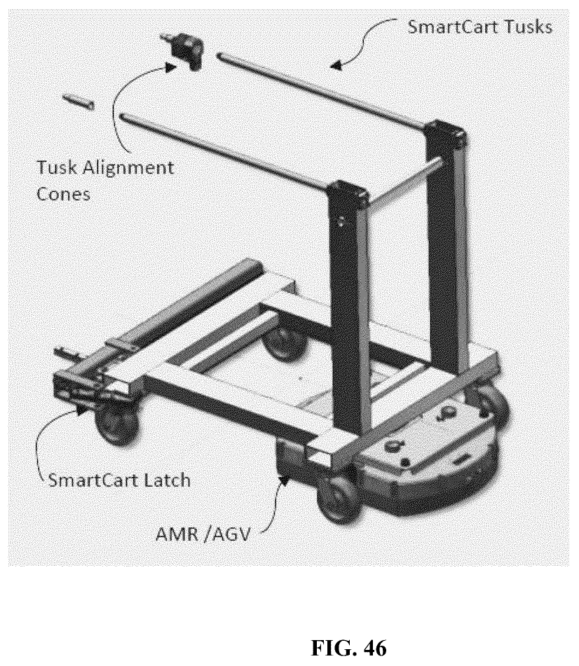

Referring to FIG. 46, an example implementation of a SmartCart can comprise: a base, such as for example a wheeled base, to facilitate mobility of the SmartCart in any direction, for example based on stored and/or received commands(s) executable by a microprocessor of ARM/AGV design; one or more tusks, or other interface mechanism(s) for docking with a station such that container(s) can be transferred between (from/to) the station and the SmartCart. In an example implementation, a latching mechanism, or a SmartLatch, can be provided for securing the SmartCart to the station, or at a location with respect to the station, to facilitate the transfer of the container(s) between the station and the SmartCart. In yet another example implementation, and alignment mechanism, such as tusk alignment cone, can be provided to facilitate the docking of the SmartCart with the station.

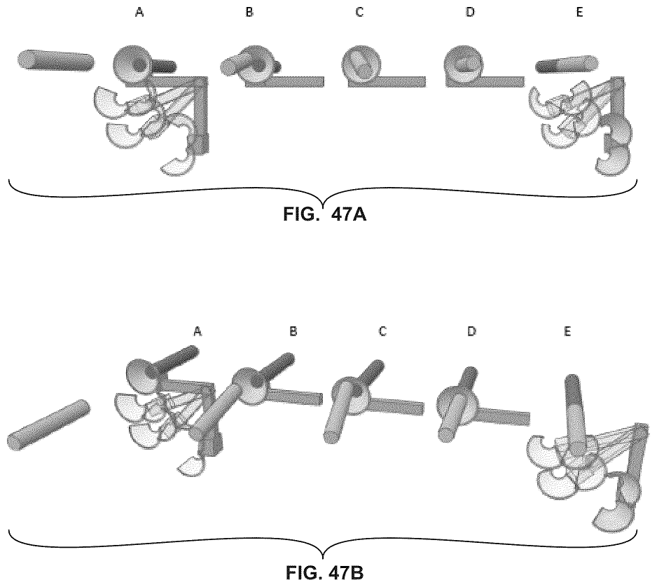

Referring to FIGS. 47A-47B, an example embodiment of the present disclosure, where SmartCart comprises tusks that interface with a station, such as SmartBagger, provides a configuration to facilitate the SmartCart tusk(s) to fully align with the SmartBagger Tusk(s). According to an example implementation, a SmartClamp Tusk Guide is configured to align SmartCart tusks and the SmartBagger tusks via, for example, cone shaped guides. As the SmartCart tusks are driven towards the SmartBagger tusks, the cone shaped guides can force the tusks to perfectly align. Once the tusks are aligned and, for example verified with sensors, the smartclamps will engage making a robust and seamless connection between the mobile bag rack and the SmartBagger apparatus, thus facilitating the automated movement of bags from the AMR driven bag rack onto the SmartBagger.

According to an example implementation, the SmartClamp Tusk Cones can be mechanically designed to articulate such that they can be lowered and raised while opening and closing to be positioned around the SmartBagger tusks. In this manner they be lowered when it is time for the containers to be loaded from the SmartCart onto the SmartBagger apparatus. The SmartClamp Tusk Cones are mounted directly to the SmartBagger Clamp Arms or SmartClamps. An example operation of an embodiment of the disclosure comprising SmartClamp Alignment Cones operation can proceed, without limitation, as follows
a. SmartClamp Arm & Alignment Cone swing into position
b. SmartCart tusks approach SmartBagger
c. SmartCart Alignment Cone redirects SmartCart tusk into proper alignment
d. SmartCart Alignment Cone properly positioned
e. SmartCart Alignment Cone swings out of position to allow Containers to be loaded onto SmartBagger

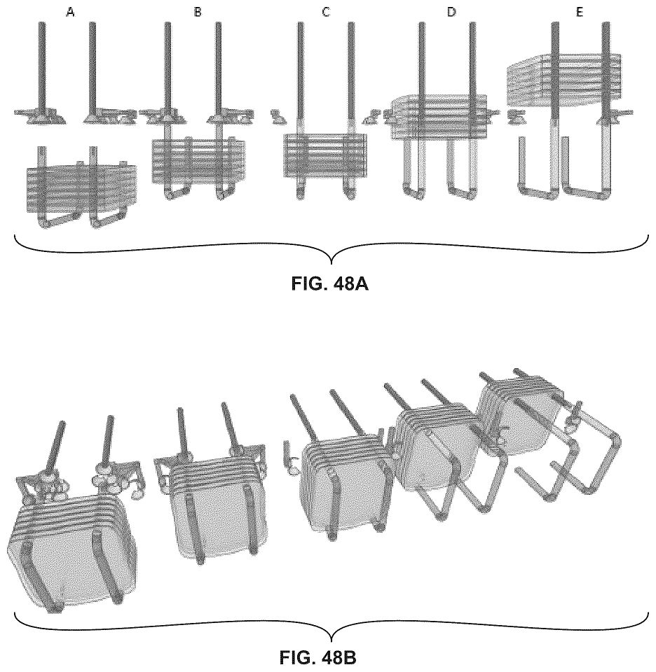

According to another exemplary implementation, a sequence of SmartClamp Cones during container loading onto the SmartBagger is shown in the example of FIGS. 48A and 48B (FIG. 48B illustrates a perspective View of a sequence of SmartClamp Cones during container loading onto the SmartBagger), where:
a. SmartClamp Arm and Alignment Cone in position as AMR/AGV driven SmartCart approaches SmartBagger
b. SmartCart tusks aligned with SmartBagger Tusks
c. SmartClamp Alignment Condes reposition to allow containers to be loaded
d. Containers are automatically loaded onto the SmartBagger
e. SmartBagger containers loaded

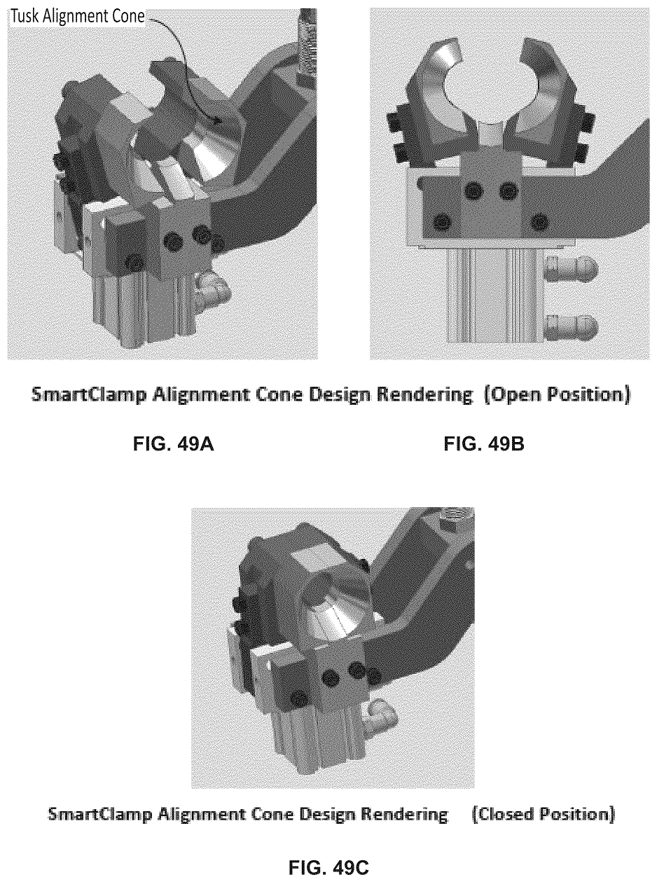

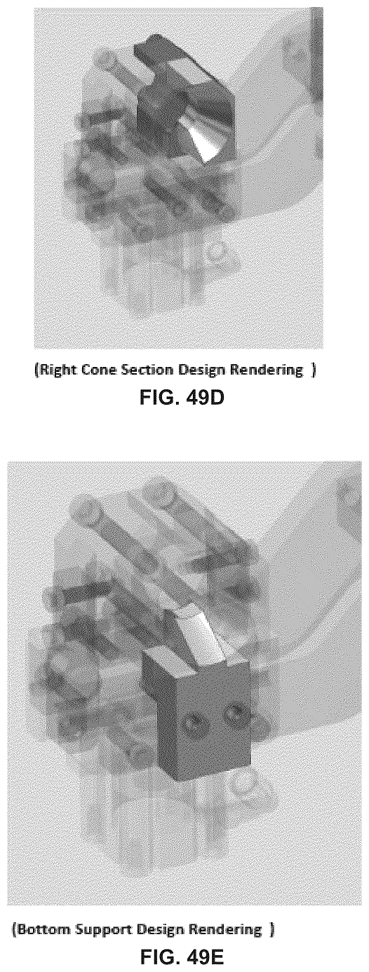

FIGS. 49A-49E illustrate certain details of example implementations of a SmartClamp according to example embodiments of the preset disclosure including, without limitation, cone design in open and closed positions, an example of a section of a cone design, and an example of a support design.

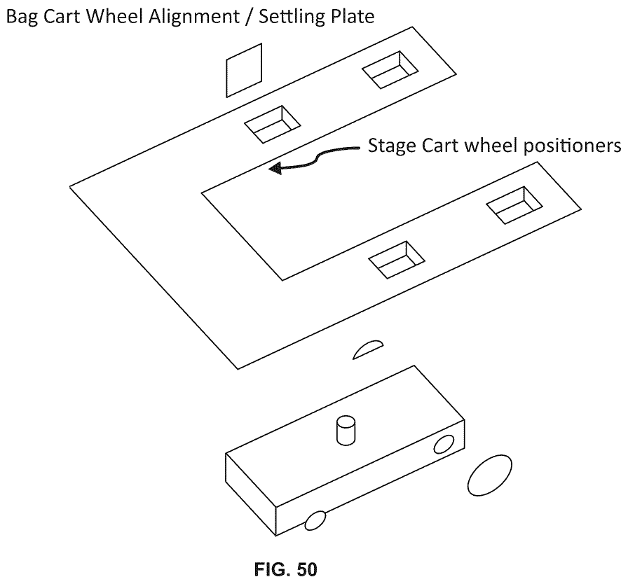

In yet another exemplary implementation, as diagrammatically shown in FIG. 50, a Bag SmartCart Floor Wheel Aligner may be used so the AMR, can autonomously drive itself into the proper location for docking. The four wheels of the AMR or cart can settle into the depressions of the floor plate. This positioning could then be sensed by the AMR or external sensors.

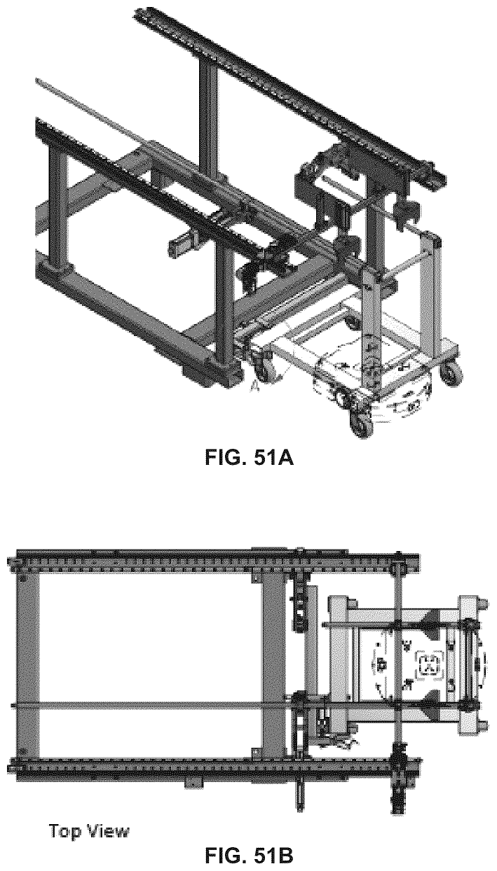

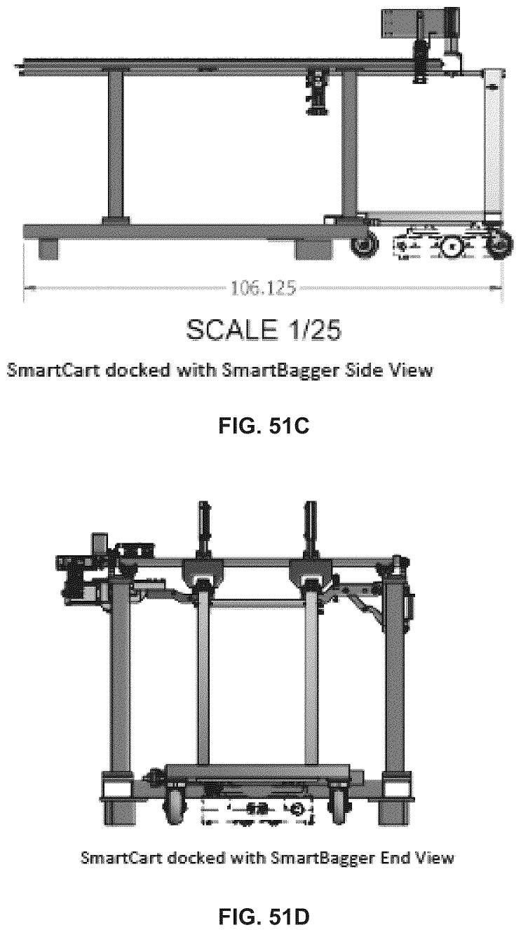

In still another exemplary implementation, as diagrammatically shown in FIGS. 51A-51D, a SmartBagger autodocking SmartCart can be designed to stage/hold empty bags, attach to an AMR/AGV system for motion and latch onto the SmartBagger apparatus to be driven by the SmartBagger into its final docking position. The cart may incorporate wheels to minimize the load required by the AMR/AVG system and provide additional stability with a full load during motion. This can also facilitate increased AMR/AGV velocity with lower risk of tipping. The cart can also have a lightweight design to also reduce the load requirements of the AMR/AGV. Furthermore, the cart can be designed to function with or without the assistance of the AMR/AVG so humans can operate the cart system to feed the SmartBagger should this become necessary.

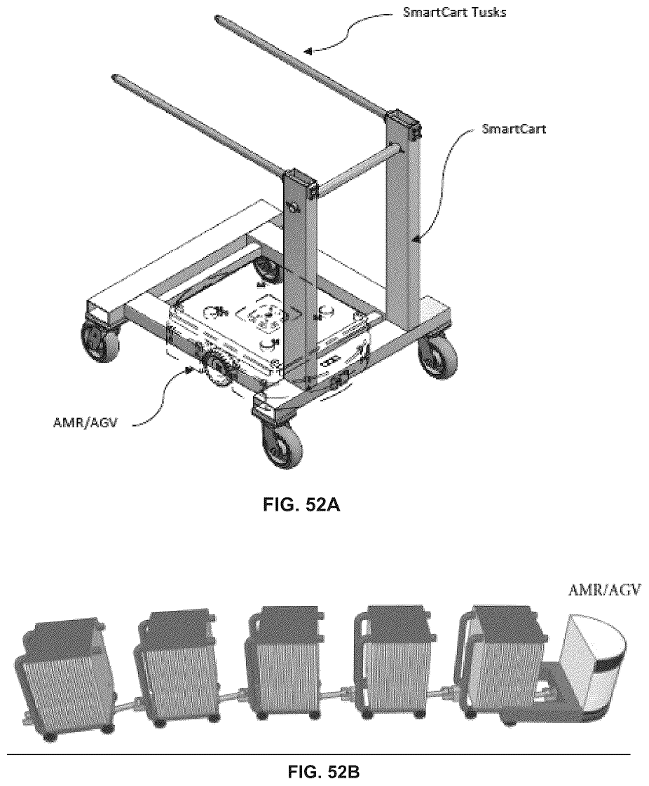

As illustrated in the example of FIG. 52A, The AMR/AGV could be designed to either tug the SmartCart by way of an automatic engagement arm/hook or to dock under the SmartCart for a more centralized, low center of gravity and more precise autonomous navigation.

As further illustrated in the example of FIG. 52B, the design of example implementations could incorporate an automated hitching system to support a train of Empty or Full SmartCarts to reduce the AMR/AGV quantity and subsequent cost of the system.

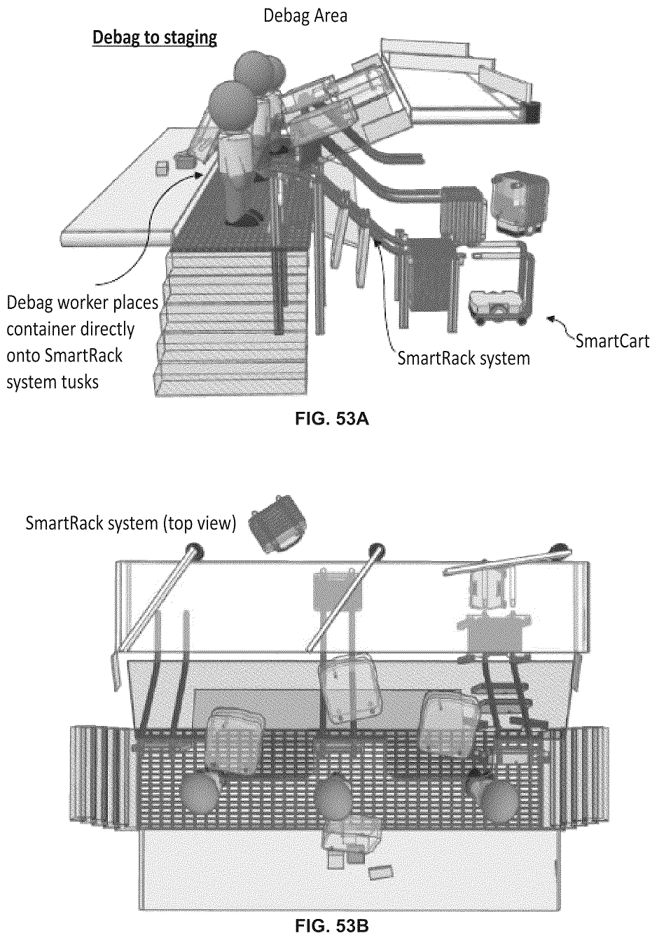

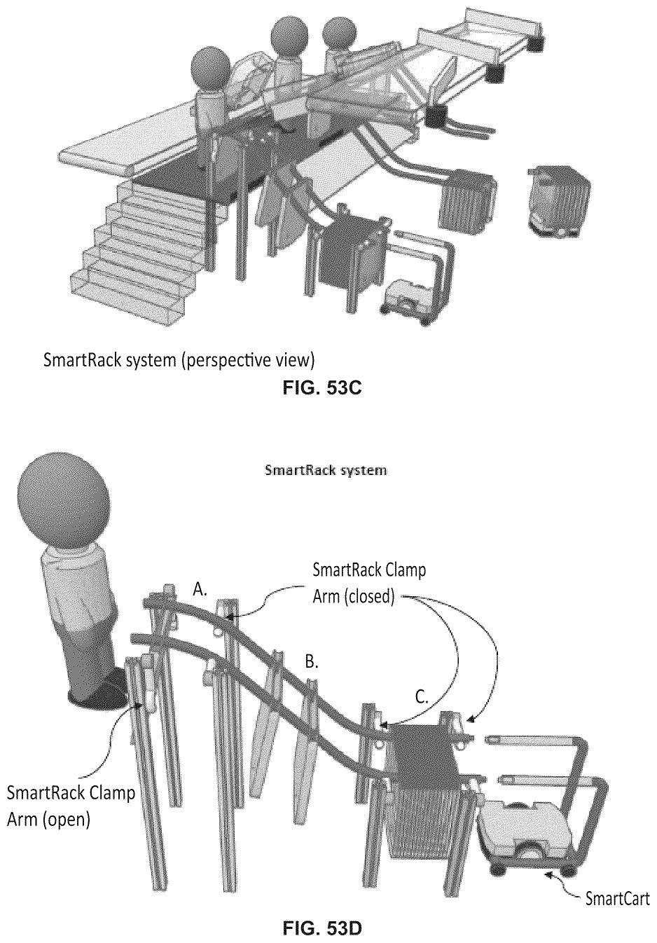

Referring to FIGS. 53A-53D, according to another example embodiment of the disclosure a mechanical or electromechanical system can be provided to allow the employees presently emptying the containers to directly load tusks at the debag area verses re-containerizing the containers or dropping them individually to subsequent works to perform the task of placing the containers onto the tusks required for, for example and without limitation, an Automated Consolidated Bagging (ACB) and SmartBagger systems. In an example implementation, the tusks can be engineered such that they directly feed the SmartCart device, thus potentially replacing the workers performing this task in present systems. Advantageously, a significant number of hours that are utilized in acquiring and reacquiring the containers to load them onto the current container stands in a non-automated system, can be reduced,

In another example implementation, SmartCarts can auto dock with the SmartRack system in a similar manner to docking with the SmartBagger apparatus. The system could then automatically grab the containers and reposition them onto the SmartCart(s).

In yet another example implementation, a series of gates or arms similar to the SmartBagger clamp arms and SmartClamps could be deployed to facilitate the movement of the empty containers from the Debag platform down to the AMR/AGV SmartCart plane. Any combination of gravity, debag worker force and/or automation can be used to drive the containers to either fill the SmartRack system or position the empty containers towards the end of the SmartRack system to be automatically staged onto the SmartCart(s).

Referring to FIG. 53D, according to still another example implementation, clamp arms can work in an alternating sequence to support the SmartRack tusks and allow empty containers to pass from zone A. (loading zone) to zone B. (transition zone) to zone C. (SmartCart loading zone) by alternating open and closed clamp arms containers are permitted to transition to the next zone while still supporting the SmartRack tusks. These may be manually or electrically actuated. An example of a process flow includes, without limitation:
(A.) The Debag worker loads containers onto the SmartRack system
(B.) Containers transition zone where containers may also be staged
(C.) Containers are automatically loaded on the SmartCarts after successful docking
   a. Mechanized system to automatically move empty containers onto SmartCarts

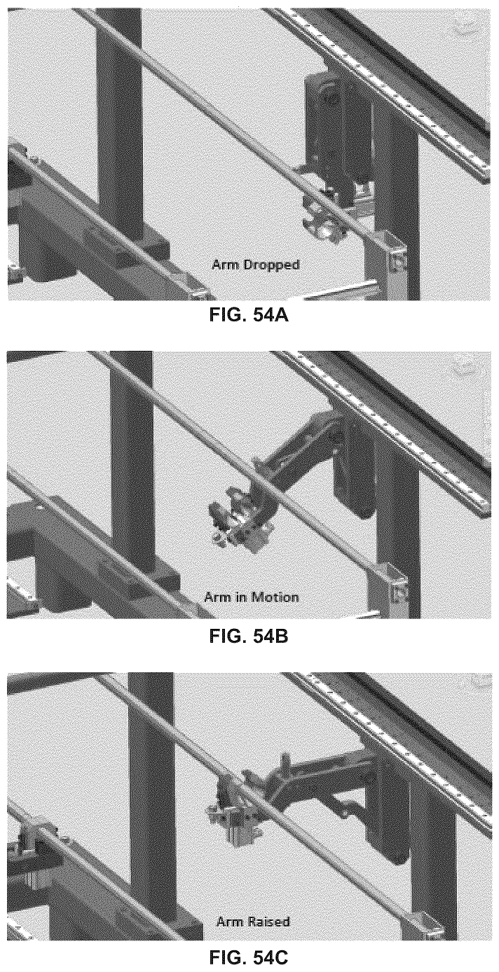

Referring to FIGS. 54A-54C, a sequence of Clamp Arm/Tusk Alignment Cone operations and associated mechanical features provided according to example embodiments of the present disclosure can be applicable to any of the systems and operations described above.

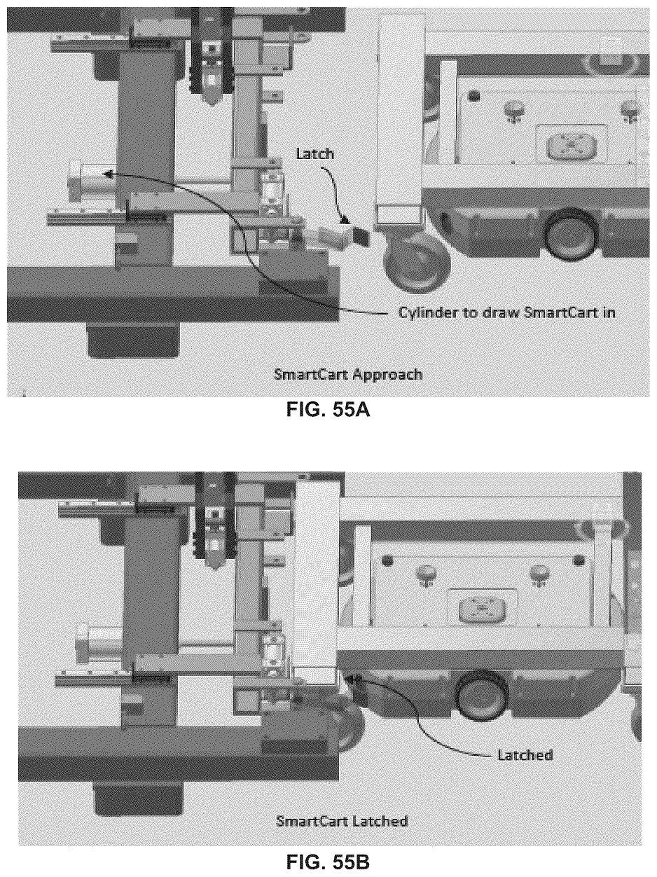

Referring to FIGS. 55A-55B, a sequence of SmartCart latching operations and associated mechanical features provided according to example embodiments of the present disclosure can be applicable to any of the systems and operations described above.

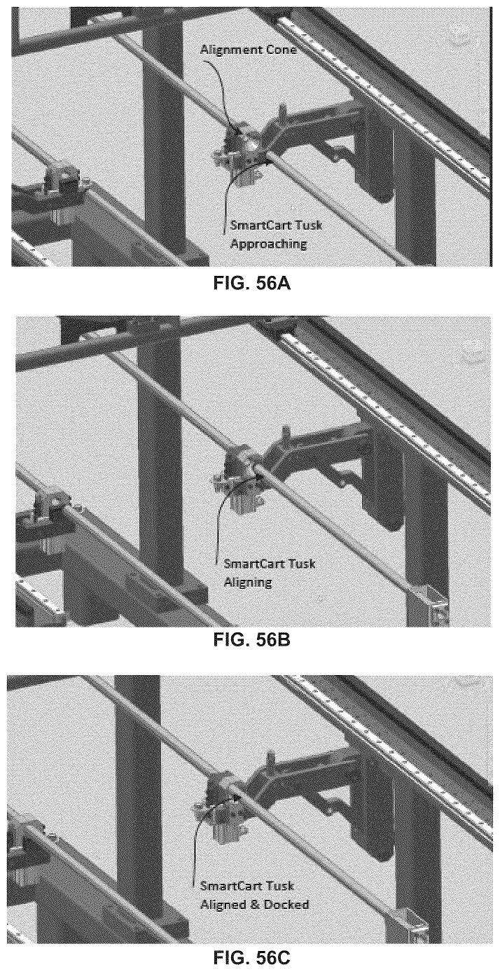

Referring to FIGS. 56A-56C, another example of a sequence of Tusk alignment and docking operations and associated mechanical features provided according to example embodiments of the present disclosure can be applicable to any of the systems and operations described above.

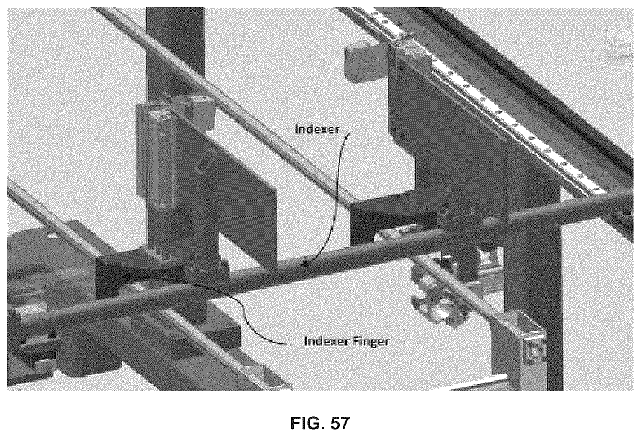

Referring to FIG. 57, an example of a SmartBagger Indexer to acquire containers from SmartCart after docking operations and associated mechanical features provided according to example embodiments of the present disclosure can be applicable to any of the systems and operations described above.

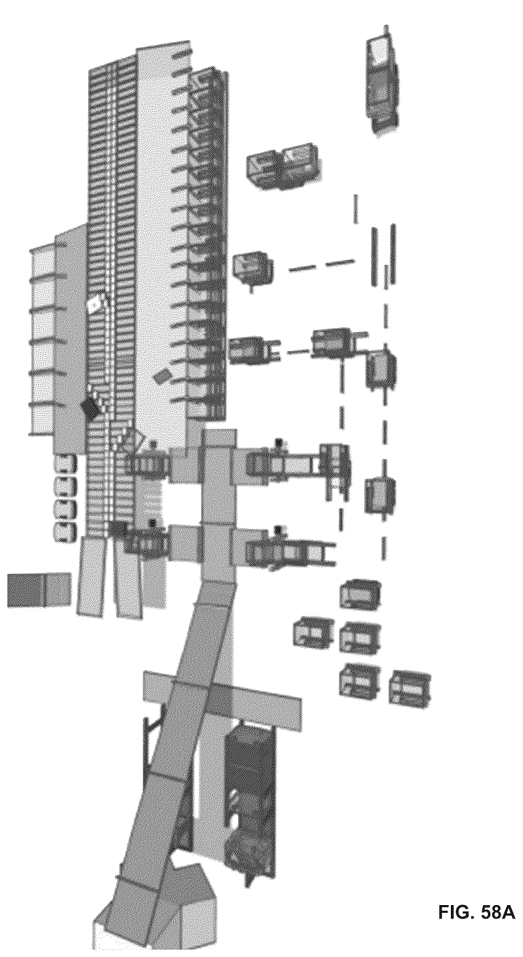

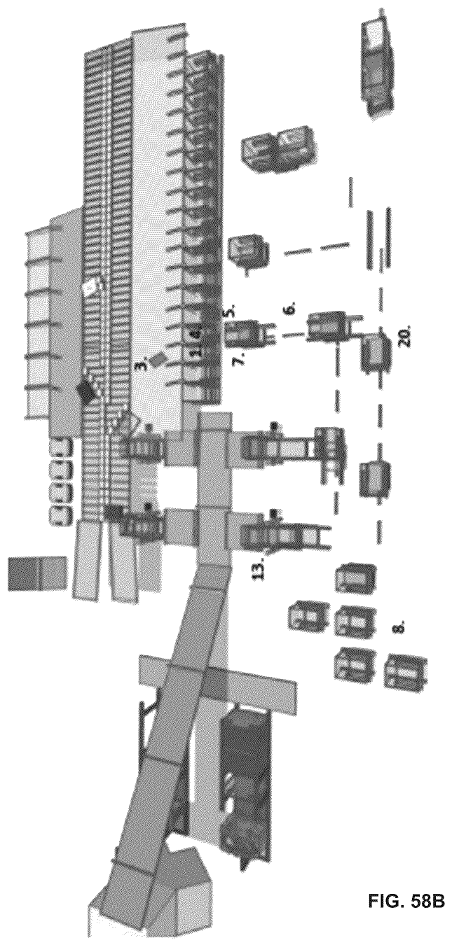

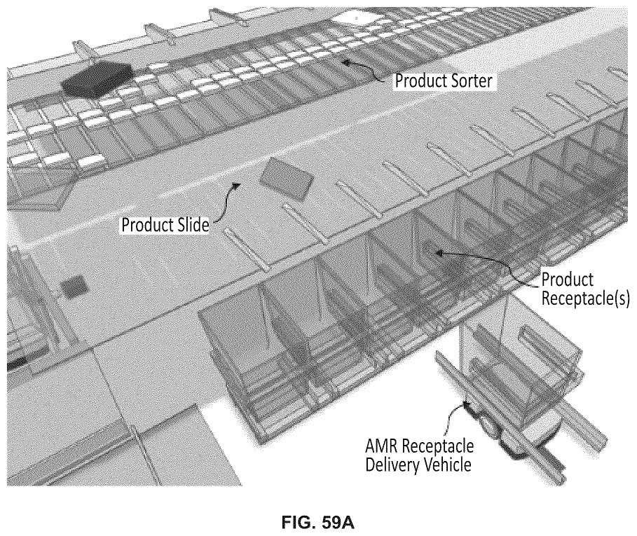

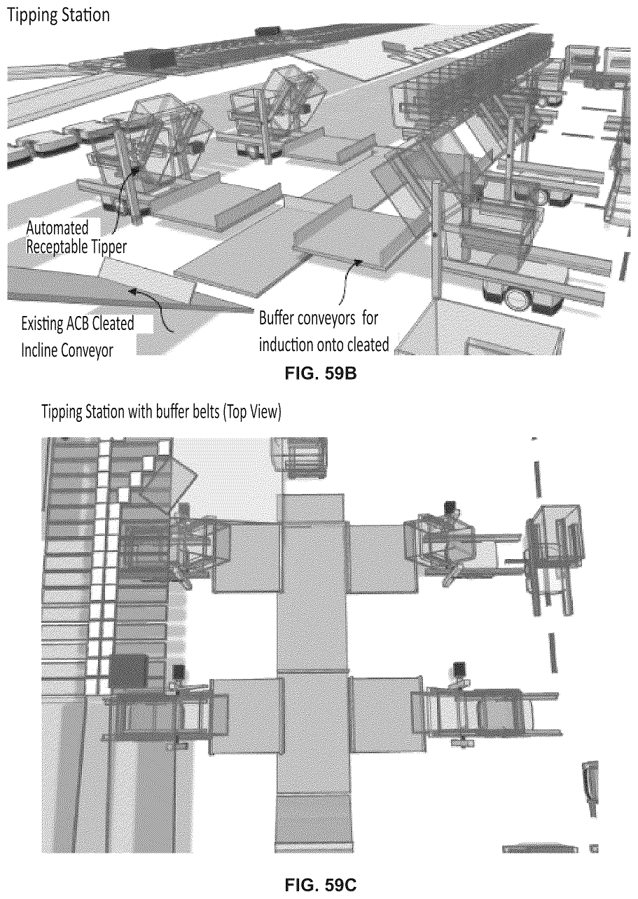

Referring to FIGS. 58A-58B and FIGS. 59A-59G, another example embodiment of the present disclosure provides an ACB system comprising a configuration where a product can be dropped directly into receptacles or open containers such as totes, for example instead of, for example into SmartBins, and then onto the collector belt. Then AMRs can be configured to collect the full receptacles, and then transport them to a tipping device onto a conveyor, for example a cleated conveyor, for further transport, for example to a SmartHopper or to a tipping device directly to the SmartBagger. The AMRs can also be configured to exchange full receptacles with empty receptacles for the sorter destination. Both full and empty receptacle staging areas can be incorporated to reduce AMR queuing to improve system performance while reducing the quantity of AMR's required and cost. In addition, segmentation of the AMR paths into zones and staging areas could be utilized to further reduce the travel path and transit time to improve performance.

According to an example implementation, as shown in non-limiting illustrations of FIGS. 58A-59G, a system can comprise:
Receptacle(s) for positioning at each destination of the sorter
   Engineered such that they can be easily acquired, staged at sorter and tipped or emptied
Cart(s) or rack(s) to support and transport the product receptacles.
Tipping station(s) to empty the receptacles onto:
   Buffer conveyor
   Cleated conveyor
   SmartBagger chute
   Other
AMR or AGV units
Controller system for AMR/AGV guidance
Controller system(s) to integrate product sorter, ACB system, AMR transport system, Tipper, Conveyors, and all other related components
AI system to dynamically improve AMR dispatches and pathway performance
Buffer belts at tipping stations to improve performance and maximize cleated conveyor zone utilization.
Empty and full receptacle staging arears
Dynamic system human interface, diagnostics, alarming, and reporting

According to an example implementation, as shown in non-limiting illustrations of FIGS. 58A-59G, an ARM sequence, including certain process steps, can comprise:

| Step # | Ref. | Events at sorter |
|---|---|---|
| 1 | A | Empty receptacle in position at Sorter Dest |
| 2 | B | ARM moves to next assignment |
| 3 | C | Sorter begins diverting parcels |
| 4 | D | Pre-notification of receptacle full |
| 5 | E | Receptacle is Full |
| 6 | F | AMR moves in place to acquire full Receptacle |
| 7 | G | AMR acquires receptacle |
| 8 | H | AMR moves receptacle to staging area |
| 9 | I | AMR with empty Receptacle in position |
| 10 | J | AMR completes moves & drops empty tote at |
| | | destination |
| | | Repeat (step 1) |
| | | Events at tipper |
| 11 | A | Tipper available |
| 12 | B | Full receptacle in position at Tipper |
| 13 | C | Tipper Tipping initiated |
| 14 | D | Tipping complete & ready for empty receptacle |
| | | retrieval |
| 15 | E | AMR in place for empty receptacle retrieval |
| 16 | F | Retrieval complete |
| | | Repeat (step 11) |
| | | Events at tipper staging drop off |
| 17 | A | AMR evaluates all tipper availability in route |
| 18 | B | AMR system determines no tipper available |
| 19 | C | AMR drops full receptacle in tipper staging area |
| 20 | D | AMR moves to new dispatch |
| | | Complete |
| | | Events at tipper staging processing |
| 21 | A | AMR system evaluates all tipper availability |
| 22 | B | AMR system is notified or determines tipper is |
| | | available |
| | | Move to step 11 |

Example implementations of systems and methodologies, as shown in non-limiting illustrations of FIGS. 58A-59G, can provide the following non-limiting or required benefits:
1. Elimination of collector belt and constraining product zones to improve containers/hr.
2. Improved performance of cleated conveyor via the buffer belts at tipping stations
3. AMR Staging areas for full receptacles to improve performance
4. Quicker and lower cost system installation
5. Lower footprint for constrained facilities
6. Possible increased product piece count per container
7. Elimination of SmartBins resulting in fewer system components and failure points
8. Elimination of pneumatic system for SmartBins

According to further example implementations (see, for example, FIGS. 59D-59G) other advantageous features can be provided in various combination, as would be understood by one of ordinary skill in the art, where for example and without limitation: a container (such as a tote) exchange can be facilitates by pushing a full tote with an empty tote (see example FIGS. 59D and 59E); a tote rack can be configured at each sorter drop position; and to take facilitate moving full totes of the rack, powered and/or free rolling take away rollers can be provided (see non-limiting example of FIGS. 59D and 59G). In an example implementation, a motion assist actuator can be configured as illustrated in a non-limiting example of FIG. 59F to assist and/or facilitate with an initial tote motion.

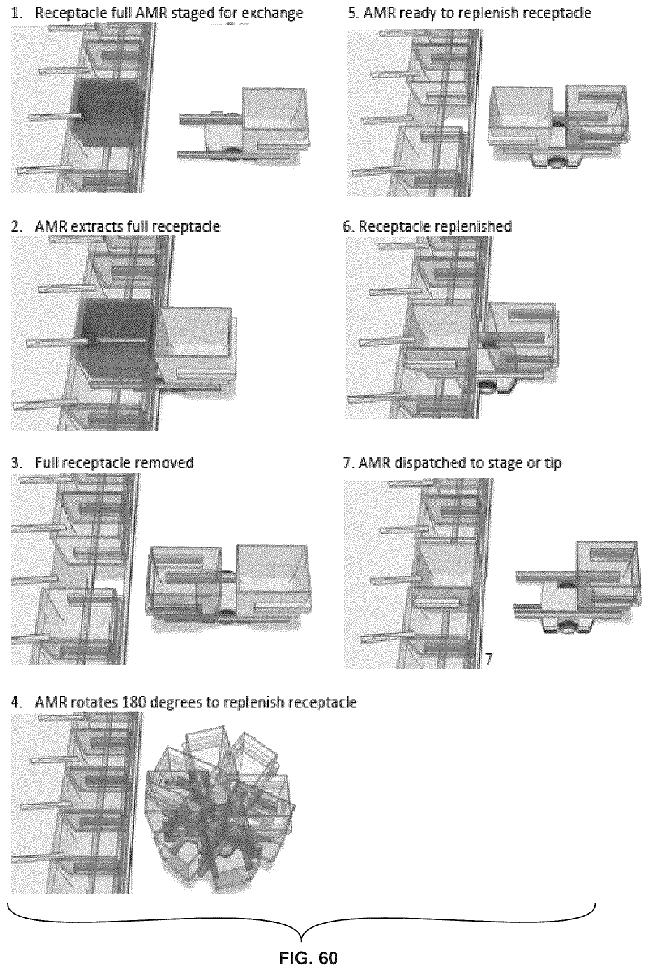

According to an example implementation, Dual Receptacle Carts can be engineered to hold two receptacles to facilitate optimized receptacles replenishment to reduce sorter destination disabled time and recirculated or re-handled product. Such a configuration can also reduce ARM/AGT unit(s) requirement and cost. As shown in non-limiting illustration of FIG. 60, a Dual Receptacle Cart Exchange sequence, including certain process steps and associated mechanical configurations can comprise:
1. Receptacle full AMR staged for exchange
2. AMR extracts full receptacle
3. Full receptacle removed
4. AMR ready to replenish receptacle
5. MR rotates 180 degrees to replenish receptacle
6. Receptacle replenished
7. AMR dispatched to stage or tip

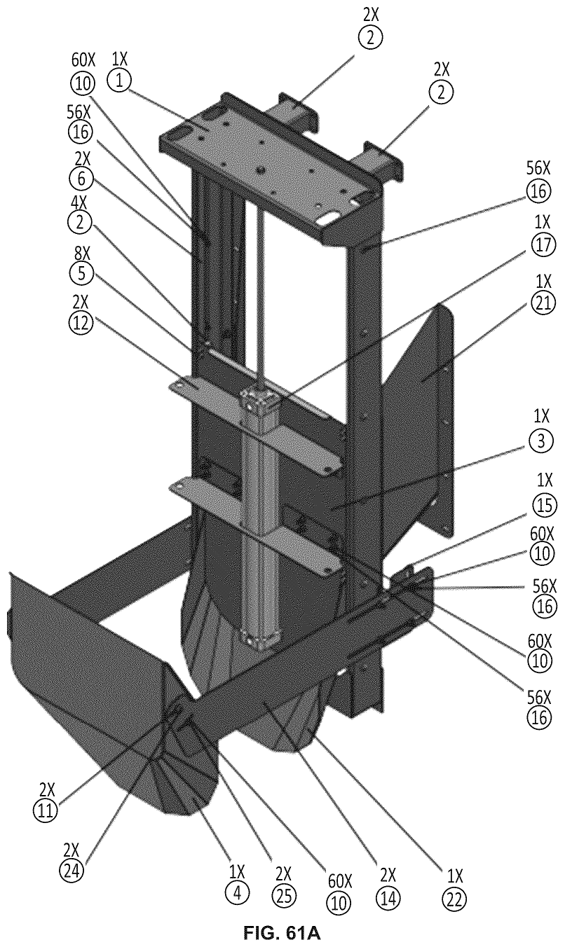

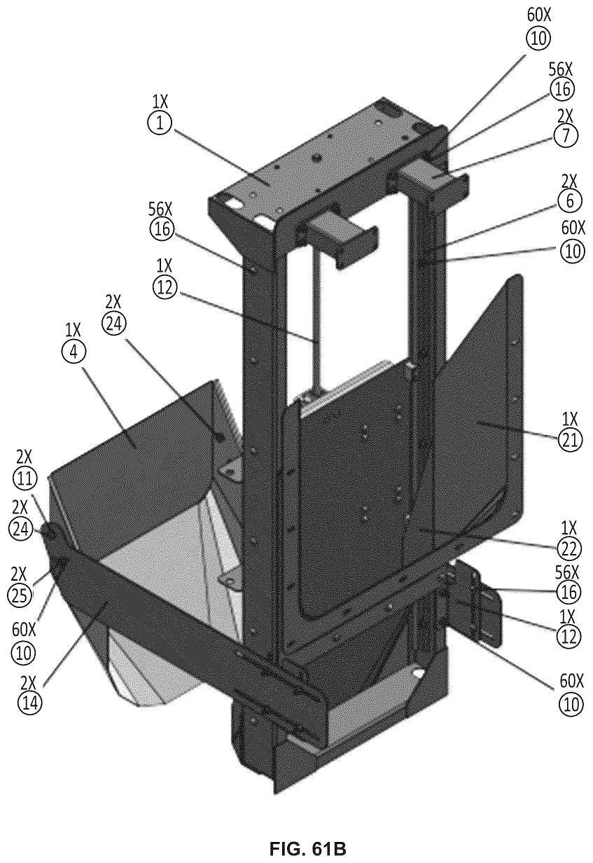

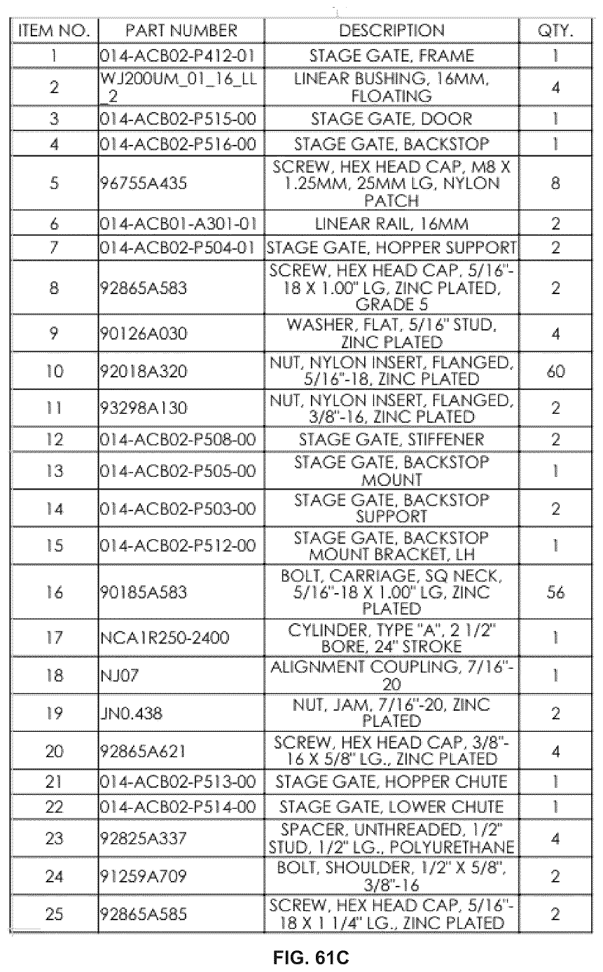

According to yet further example embodiments of the present disclosure, to increase the performance of the SmartBagger system, the fill position processing time can be improved. Referring to FIGS. 61A-61C, which illustrate in system drawings an example implementation of a mechanical stage gate, “StageGate,” according to an example implementation (where FIG. 61C describes labels utilized in FIGS. 61A-61B), a system, such as an ACB, can incorporate an open chute design to fill the containers. In an example configuration, due to the length and angle of the chute, the package travel time is measured at approximately 4.5 seconds from the top of the Smart Hopper where the cleated conveyor adjoins, and the exit of the chute. In addition, without the introduction of a SmartGate, the SmartBagger fill processing time also includes the cleated conveyor run out time from the time the volume is requested by the Smart Bagger. To remedy this conveyor and package travel time, a mechanism to stage the packages at the bottom of the chute can be provided. If an automated gate is positioned at the exit of the chute this can provide multiple benefits, such as for example and without limitation:
1. The packages will have less distance to travel, thus reducing the time to fill the containers
2. This gate will add a package zone buffer to each of the chutes of the SmartHopper which will in turn enable the ACB cleated and collector conveyors to remain running. This will improve the performance rate and reduce OTE, off the end, packages on the sorter reducing system defects, or re-handles.
3. This gate will dampen the effects of gravity and inertia on the packages hitting the bottom of the container, or each, other thus improving the package care implementation of the SmartBagger system
4. Incorporating a backstop and sides will also reduce the number of packages missing the container.

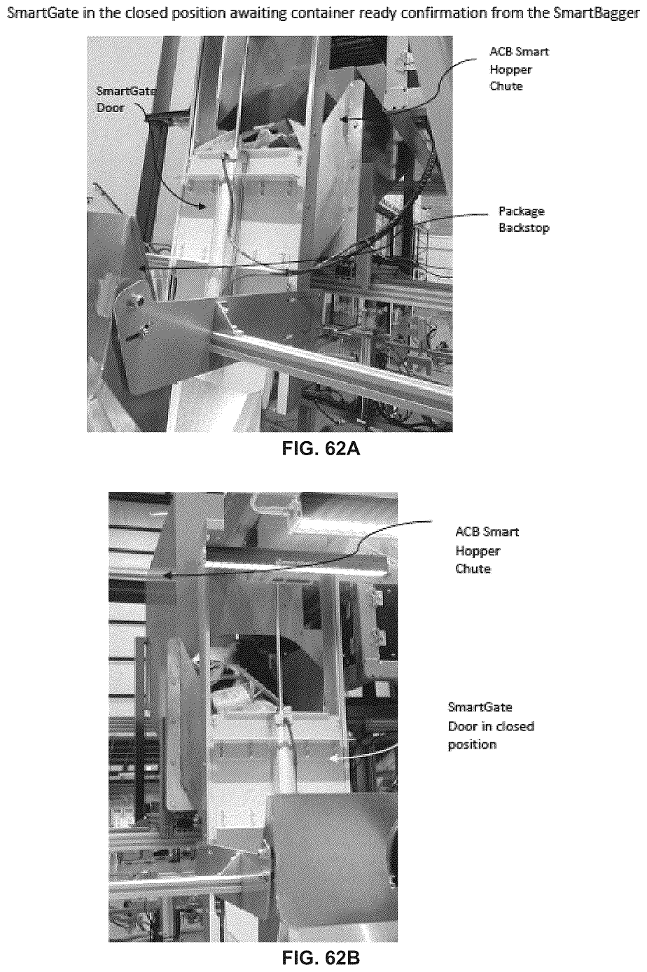

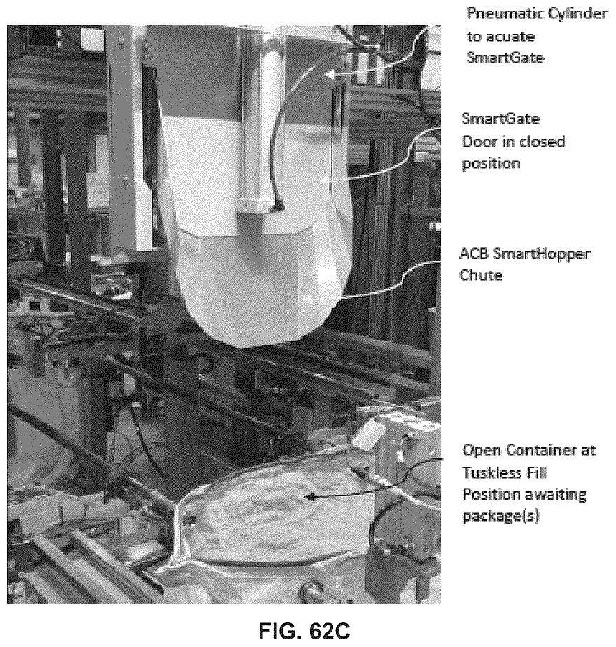

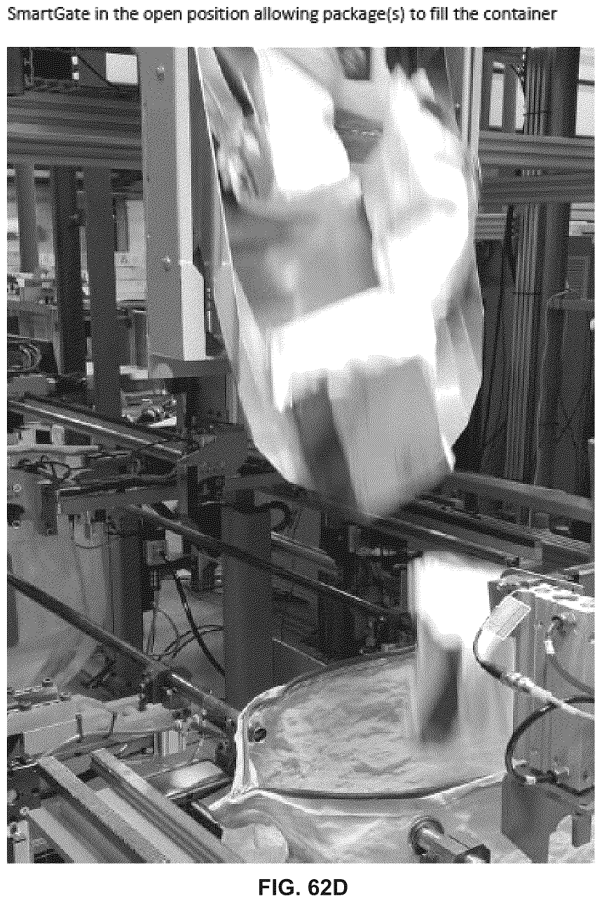

Referring further to FIGS. 62A-62C, according to example implementations of the disclosed embodiments, a system, such as an ACB system, can flow packages into each of the chutes on the SmartHopper with the gates closed. When the SmartBagger system is ready to receive the packages into the container, it will open the gate to release the packages. After the release of packages, the system, for example using a vision processing unit, VPU, can be configured to inspect the chute, gate, and container to ensure the gate can be closed. Upon confirmation of a safe to a close state, the Smartbagger system can initiate a SmartGate close command, and the gate can be closed and ready for the next batch, for example of an ACB zone, of packages from the feeding conveyor, for example a cleated conveyor.

In yet another example implementation, in addition to the SmartGate door, a backstop and sides located in the gap between the chute/SmartGate exit the container opening can be introduced to prevent packages from missing the container.

In yet further example implementation, SmartGates can be engineered to open vertically or horizontally depending on the system design requirements.

In still further example implementation, SmartGate in the closed position can remain in the closed position awaiting container ready confirmation from the SmartBagger.

Other example non-limiting benefits of AMR Based ACB solution where AMRs and containers/totes can be used versus Smart Bins and/or collector belts include:
Quicker Cheaper install
Smaller footprint at sorter for constrained areas
Increased parcel/product capacity
Cost effective distribution of work areas in system layout (Sorter versus Bagging)

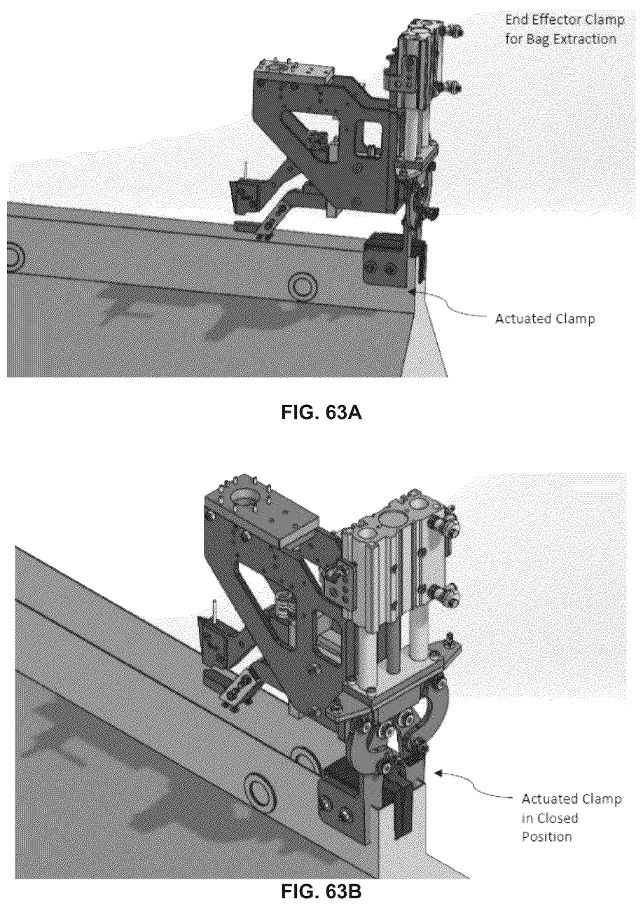

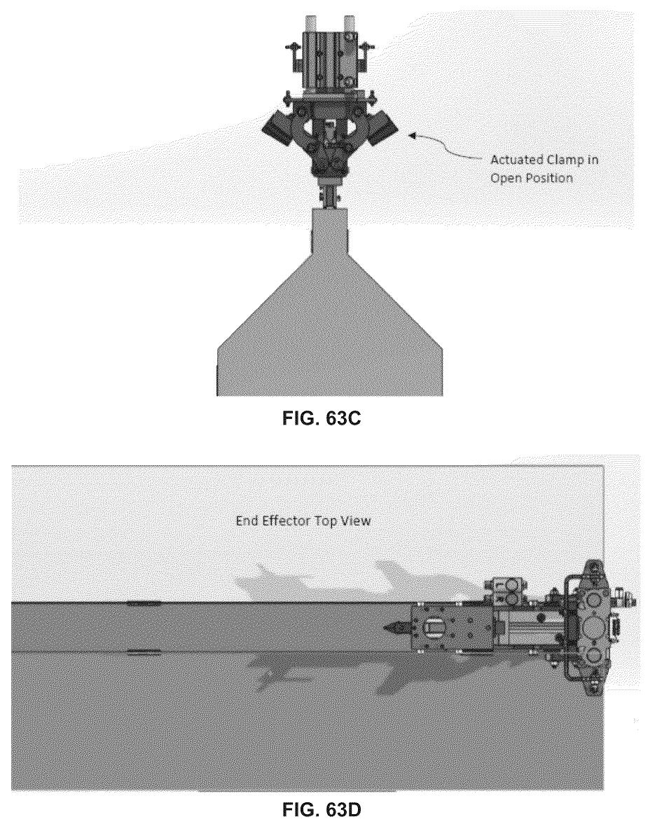

Referring to FIGS. 63A-63D, according to another example implementation of example embodiments of the disclosure provide an automated assist for removal of containers from container closing location, such as for example a robotic assist for removal of a bag from a Zipping station. For example, a robotic assist can be utilized to increase the performance of the removal of the bag from the Zipping station, which can for example prevent top heavy containers from falling over, prevent light weigh containers from slipping on the conveyance system and increase the speed containers exit the system. In further example implementation, a robotic motion could be used to pick and place containers onto several targets. For example, such targets could be for buffering full container flow, post processing of containers or exception handling of the containers.

In yet further example implementation, a zipping end effector can be modified with a clamping mechanism to grip the Bag for the extraction motion. A vision system, VPS, can be configured to inspect the container after the competing zipping attempt, to determine the x, y, z coordinates of the optimum gripping location. The VPS can be further configured to then provide these coordinates to the robotic controller and execute the gripping motion. The VPS can be configured still further to then verify the container has been gripped. The SmartBagger controller can be configured to then determine the placement point of the container and instruct the robotic controller to move the container to the appropriate location. According to sill further example implementation, once the robotic controller has verified the completed motion, the gripper can be opened, and the container released. The VPS system can be further configured to then verify the container is in the proper location.

FIGS. 64A and 64B are a perspective view and a side view, respectively, of a chute tendering system 600 for chute tendering and container fill applications according to one or more example embodiments.

Referring to FIGS. 64A and 64B, chute tendering may be required when parcels are stuck or jammed in a chute 601 (may also referred to as a hopper), such as a SmartBagger chute. For example, parcels can be manipulated by various means to free the parcels to allow them to freely flow into a container 608. In an example implementation, opposing bidirectional conveyors 602, 603, positioned at an angle along facets of the chute 601, can be activated to free the parcels. For example, conveyors 602, 603 can be implemented to use high coefficient of friction belting, such that the parcels can be lifted upward and/or can be pulled downward, depending on, conveyor direction for example. Such an action can help facilitate the freeing of jammed parcels to resume the gravity free fall into the container 608.

In an example implementation, a parcel jam detection system comprised of a variety of sensors 612 which may be discrete or vision/perception, can be provided to detect parcels which are jammed in the hopper. Such sensor(s) may be positioned within the chute 601 at any of locations 612a, 612b, and 612c, or any another location, either within or outside of the chute 601, as would be understood by one of skill in the art. A system controller, such as a SmartBagger system controller, can be operatively connected to the sensor(s) 612 and configured to initiate tendering conveyors 602, 603 based on information from a parcel jam detection system including the sensor(s). Such action can be implemented in any of various profiles relating to, for example, any one or more of combinations of conveyor velocity, frequency, and direction. In another example implementation, the parcel jam detection system can continue to monitor the parcel jam condition and notify the system controller when the jam has been cleared. In another example implementation, an artificial intelligence system may be incorporated to determine the most efficient conveyor motion profiles to utilize based upon the detected jam parcel-positioning and configuration(s) to achieve the most reliable and expeditious results.

According to an example implementation, in addition to the opposing conveyors 602 and 603, cylinders with rods 609 and 610, or similar mechanical devices, can be added to actuate and impact or poke the parcels in a way that will assist in freeing the parcel jam. This may be completed, for example, in conjunction with the opposing conveyors 602 and 603.

According another example implementation, to prevent parcels from missing the container(s), guides or flaps 605, 606 can be engineered to extend into the container using an actuator 604. For example, prior to the release of parcels into a hopper, the guides, or flaps 605, 606 may be extended or in an extended position. Once the container fill process is complete, the guides or flaps 605, 606 can be retracted to allow the container to move along the rods, such as SmartBagger rods, to the next station, such as to the SmartBagger processing station. In the examples of FIGS. 64A and 64B, the guide or flap 605 is shown in a retracted position, while guide or flap 606 is shown in an extended position. In an example implementation, the guides or 605, 606 can be fully extended or fully retracted in unison.

FIGS. 65A-65E illustrate a system 700 for an ACB/SmartBagger bilateral AB splitter according to one or more example embodiments.

Referring to FIGS. 65A-65E, in order to accommodate the parcel feeding of multiple systems 701, 702 such as SmartBagger systems, at a required rate, a system comprised of multiple conveyors 704, 705 such as SmartBagger hopper feed conveyors, can be placed perpendicular to a bagging feed conveyor 706, such as the ACB, automated consolidated bagging feed conveyor. In an example implementation, hopper feed conveyors, such as the SmartBagger hopper feed conveyors, can be configured to feed work in tandem to provide parcel volume to a system, such as to the SmartBagger systems. In a further example implementation, the system can also be configured with additional conveyors to feed additional system(s), such as additional SmartBagger system(s).

According to an example implementation, the system may include one or more sensors 712 positioned, for example at one or more of locations 712a, 712b, 712c, and 712d, and configured, for example, to sense one of a volume and a number of parcels disposed on one or more of the hopper feed conveyors. The sensors 712 may be operatively connected to a controller, such as a SmartBagger system controller, and the controller may be configured to control tendering of the hopper feed conveyors based on information from the sensors 712. For example, the controller may be configured to hold a hopper feed conveyor until a predetermined volume or number of parcels is positioned thereon, and, upon determination that the predetermined volume or number has been reached, may be configured to control the corresponding hopper feed conveyor to move the parcels thereon onto the bagging conveyor. In another example implementation, the system can continue to monitor the volume and/or number of parcels on a hopper feed conveyor and notify the system controller when the predetermined volume or number has been reached. In another example implementation, an artificial intelligence system may be incorporated to determine the most efficient conveyor motion profiles to utilize to achieve the most reliable and expeditious results.

According to another example implementation, in addition to directing the parcels to multiple systems, such as SmartBagger systems, the system can be engineered to provide a buffer or parcels to improve overall system performance, for example ACB/SmartBagger system performance. For example, the required longer parcel zones on a collector conveyor, such as ACB parcel zones on an ACB collector conveyor, can be consolidated onto shorter conveyors 704, 705, such as shorter SmartBagger hopper feed conveyors. This can reduce the parcel feed time to a chute, such as to the SmartBagger chute. In addition, by staging parcel volume on conveyors 704 and 705, such as on SmartBagger hopper feed conveyors, automated consolidated bagging performance, such as the ACB performance, can be increased as, for example, the collector belt, such as ACB collector belt, may require fewer stops to wait on a container fill processing, such as the SmartBagger container fill processing.

In yet another example implementation, depending upon the flow splitter conveyor configuration, such as a ACB flow splitter conveyor configuration, a flapper 710 can be provided to prevent parcels from spilling onto the adjacent conveyor. Referring to FIG. 65D, the example flapper 710 is shown in an open position 710a, and in a closed position 710b. In an example implementation, a flapper actuator 712 can be controlled by a control system such as a SmartBagger controls system. For example, depending upon which, for example SmartBagger, conveyor 704 or 705 is available, the flapper 710 can be adjusted to either the open 710a or closed 710b position.

In still another example implementation, parcel stop flaps 706, 707 can be provided to prevent parcels from overshooting chute(s) 708, 709, such as SmartBagger chute(s), during the transfer of parcels from conveyors 704, 705, such as SmartBagger hopper feeder conveyors.

FIG. 66 is a perspective view of system of conveyors, according to one or more example embodiments, to isolate exception bags, requiring human intervention, from the processed bags.

Referring to FIG. 66, an automated mechanical system such as a diverter or plunger can be activated by a control system, such as the SmartBagger control system, to divert any exception bag to a conveyor, roller, or slide to an accumulation area 803. In an example implementation, the control system, for example SmartBagger control system, can track the progression of the bags on a conveyor 801, for example on SmartBagger container take away conveyors, by reading indicia on the bag label, reading container embedded RFID tags, and/or photocell conveyor/item tracking logic. For example, when an exception bag is present at the actuator the bag can be diverted at location 802, to the accumulation area 803.

In still further exemplary implementations of bagging systems described in the present specification, such as for example and without limitation, a system comprising SmartBagger features and components illustrated in FIGS. 64A-66, one or more vision system(s), comprising for example and without limitation:

AI Cameras, can be added to detect: for example and without limitation:
Bag overfills
Packages stuck in fill chute
Empty Bag in position
Grommet gripping success

Volume metrics on the sorter can be implemented to, for example and without limitation:
Prevent bag overfills, where, for example,
   The smalls sorter camera system provides parcel dimensions for each parcel which are used to calculate the total volume of parcels in the ACB Bin. This is used to ensure the parcel volume does not exceed a threshold set for filling the bags on the SmartBagger system.
   Improves Zipping
   Reduces human interventions & improves throughput
Include the ability to restrict oversize parcels, which for example
   Increases parcels per bag KPI
   Reduces human interventions & improves throughput
Add bag tendering actuator profiles to vary motion to improve packages settling into the bags, which for example, can reduce human interventions & improves throughput.

Referring to FIGS. 67A-69, an exemplary embodiment of the present disclosure provides a spring loaded rods and/or dogs that can facilitate prevention of tearing of grommets out of bags due to, for example, manufacturing variability of bags with regards to full opening position. For example, if a bag is stitched such that is does not open as far as it should the grommet can be ripped out of the bag material. According to example implementations of disclosed example embodiments, a spring and/or another flexile configuration, can be provided to facilitate limiting of a tension on the bag grommet, which may preventing damage to the bag and potentially limit human interventions, which may reduce system throughput.

As illustrated in non-limiting example of FIGS. 67A and 67B, rod dogs configured to hold bag grommets in position can be provided with, for example, an spring tensioner to facilitate variable opening distance and force, such that for example, where a first bag, as shown in the example of FIG. 68A (showing diagrammatically a view of an opening of a conventional empty bag) manufactured such that it can be opened further than a second bag shown in the example of FIG. 68B (showing diagrammatically a view of a more restricted or smaller opening of an empty bag due to for example a manufacturing defect), the spring action of the rods/dogs can facilitate prevention of the bag grommet from being ripped out of the bag during the opening (see example illustration of FIG. 69).

While example implementations have been shown and described with reference to certain example embodiments, it will be understood by those skilled in the art that various changes in form and details may be made therein. For example, any of various communication protocols can be deployed in combination with any of various electronic sensors, and/or any of various visual and/or audio user interfaces can be implemented to facilitate processing and/or displaying information and/or controlling hardware and/or software components of example systems.

It may be understood that the example embodiments described herein may be considered in a descriptive sense only and not for purposes of limitation. Descriptions of features or implementations within each example embodiment may be considered as available for other similar features or implementations in other example embodiments.

While example embodiments have been described with reference to the figures, it will be understood by those of ordinary skill in the art that various changes in form and details may be made therein without departing from the spirit and scope as defined by the following claims.

What is claimed is:

1. A chute tendering system comprising:
chute comprising a passage extending from an upper opening to a lower opening, wherein a cross-sectional area of the lower opening is smaller than a cross-sectional area of the upper opening;
a pair of bidirectional conveyors, each disposed along a facet of the chute between the upper opening and the lower opening.

2. The chute tendering system according to claim 1, further comprising:
at least one rod movably connected to the chute such that the at least one rod is movable between a first position in which it is not positioned within a path of parcels within the chute and a second position in which the rod extends into the path to thereby contact one or more parcels within the chute.

3. The chute tendering system according to claim 1, further comprising:
a pair of flaps movably connected to the chute, wherein each of the pair of flaps is moveable between a retracted position outside of a path of parcels exiting the lower opening of the chute and an extended position intersecting the path to thereby guide one or more parcels exiting the lower opening of the chute.

4. The chute tendering system according to claim 1, further comprising:
a parcel jam detection system comprising:
at least one sensor configured to detect at least one of a velocity, a frequency, and a direction of movement of packages within the chute;
a controller comprising a non-volatile memory storing instructions thereon, and a processor configured to execute the instructions, wherein the controller is operatively connected to the pair of bidirectional conveyors and to the at least one sensor and is configured to execute the instructions to thereby:
receive information output from the at least one sensor, and
control operation of the pair of bidirectional conveyors based on the information output from the at least one sensor.

5. The chute tendering system according to claim 4, wherein the controller is further configured to:
determine when a parcel jam has been cleared based on the information output from the at least one sensor, and
output a notification upon a determine that a parcel jam has been cleared.

6. An automated consolidated bagging system comprising:
a plurality of collector conveyors;
a bagging conveyor;
a plurality of feed conveyors, each positioned to receive parcels from a corresponding one of the plurality of collector conveyors and output the parcels onto the bagging conveyor;
at least one sensor configured to sense one of a volume and a number of parcels disposed on each of the plurality of feed conveyors;
a controller comprising a non-volatile memory storing instructions thereon, and a processor configured to execute the instructions, wherein the controller is operatively connected to each of the plurality of feed conveyors and to the at least one sensor and is configured to execute the instructions to thereby:
receive information output from the at least one sensor, and
control operation of each of the plurality of feed conveyors based on the information output from the at least one sensor.

7. A bag exception system comprising:
a take away conveyor configured to convey bags thereon;
a diverter configured to divert one or more bags off the take away conveyor;
a reader configured to read data from the bags on the take away conveyor
a controller comprising a non-volatile memory storing instructions thereon, and a processor configured to execute the instructions, wherein the controller is operatively connected to the reader and to the diverter and is configured to execute the instructions to thereby:
receive information output from the reader, and
control operation of the diverter based on the information output from the reader.

8. The bag exception system according to claim 7, wherein the reader comprises one of a camera configured to obtain an image of information on the bags and a radio frequency reader configured to read radio frequency identification (RFID) tags connected to the bags.
